(19) United States
(12) Patent Application Publication
(10) Pub. No.: US 2023/0391727 A1
(43) Pub. Date: Dec. 7, 2023

(54) ANALOGUES OF PENTAMIDINE AND METHODS FOR TREATING INFECTIONS

(71) Applicant: Auransa Inc., Palo Alto, CA (US)

(72) Inventors: Andrew Asher PROTTER, Palo Alto, CA (US); Gregory R. LUEDTKE, Salinas, CA (US); Pek Yee LUM, Palo Alto, CA (US); Anna HALBERG, Palo Alto, CA (US)

(21) Appl. No.: 18/033,320

(22) PCT Filed: Oct. 22, 2021
(86) PCT No.: PCT/US2021/072002

§ 371 Date: Apr. 21, 2023

Related U.S. Application Data
(60) Provisional application No. 63/104,455, filed on Oct. 22, 2020.

(51) Int. Cl. C07D 217/22; C07D 207/09; C07C 257/18; A61P 31/10; A61P 31/04

(52) U.S. Cl. CPC C07D 217/22; C07D 207/09; C07C 257/18; A61P 31/10; A61P 31/04

(57) ABSTRACT

The present disclosure provides a group of amidine analogs and their pharmaceutically acceptable salts that are useful for treating a bacterial or fungal infection. The infection may include those infections caused by gram negative bacteria, gram positive bacteria, or fungi. Compositions, methods of synthesizing the same and methods for treating bacterial or fungal infection are disclosed herein.

### CROSS REFERENCE TO RELATED APPLICATIONS

This application claims the benefit of U.S. Provisional Patent Application No. 63/104,455, filed Oct. 22, 2020, which is hereby incorporated herein by reference in its entirety.

### FIELD OF THE INVENTION

The present invention relates to compounds and methods useful for the treatment of human diseases, particularly against bacterial infections, fungal infections, and cancer.

### BACKGROUND OF THE INVENTION

Treatment of gram negative bacterial infections is one of the most challenging areas in medicine because of high levels of multidrug resistance developed by bacterial strains, often in hospital settings. Although the rapidly emerging threat for hospitalized patients and the need for developing new agents to treat these resistant pathogens have been well recognized, the progress remains extremely slow with only a few new drugs being developed every year. In recent studies, pentamidine has been demonstrated to be effective in sensitizing a subset of gram negative bacterial strains in vitro (Stokes et al., “Pentamidine sensitizes Gram-negative pathogens to antibiotics and overcomes acquired colistin resistance” Nat Microbial. (2017) 2:17028).

Pentamidine, 1,5-bis(4-amidinophenoxy)pentane, came into medical use in 1937 and is on the World Health Organization's List of Essential Medicines as an antiprotazoal/antifungal agent for treating various infectious diseases (e.g., African trypanosomiasis, leishmaniasis, babesionsis, and Pneumocystis carinii pneumonia). However, numerous side effects have greatly :limited the use of pentamidine against parasitic infections. Therapy involving this compound often requires careful monitoring on adverse events and dose responses. Particularly among its side effects, patients under pentamidine therapy commonly exhibit transient elevation of serum liver transaminases (e.g., ALT and AST liver injury markers). Due to these potentially harmful consequences on vital organ(s), dosage of this drug has been severely limited.

Pentamidine can be administered by aerosol, intramuscularly (IM) or intravenously (IV). Aerosol and IV treatments are the recommended route for treating infectious diseases. This is because the compound suffers greatly from poor oral bioavailability. Some studies have shown that the toxic side effects may be decreased if the drug is given by aerosol administration. Various approaches, such as pentamidine prodrugs, have been taken to overcome the compound's shortcomings in oral bioavailability, but there is no pentamidine analog reported to date that provides a safe and effective exposure at therapeutic levels.

Given the known side effects of pentamidine and the rapidly emerging threat of multidrug-resistant bacterial and fungal infections, there is a need for safe and effective, non-toxic pentamidine analogs that exhibit increased potency and synergy against bacterial and fungal strains.

### BRIEF DESCRIPTION OF THE DRAWINGS

FIG. 1 depicts images of multi-well plates demonstrating checkerboard assay results using rifampicin and pentamidine alone and in combination; and rifampicin and compound 3 alone and in combination against Klebsiella pneumoniae in indicated drug concentrations (μg/mL).

FIG. 2 depicts images of multi-well plates demonstrating checkerboard assay results using novobiocin and pentamidine alone and in combination; and novobiocin and compound 3 alone and in combination against Acinetobacter baumanii in indicated drug concentrations (μg/mL).

FIG. 3 depicts checkerboard assay results using novobiocin and compounds 1, 2 and 3 alone and in combination against A. baumanii in indicated drug concentrations (μg/mL).

FIG. 4 depicts an image of a multi-well plate showing a checkerboard assay result using rifampicin and compound 2 alone and in combination against K. pneumoniae in indicated drug concentrations (μg/mL).

FIG. 5 depicts an image of a multi-well plate showing a checkerboard assay result using novobiocin and compound 7 alone and in combination against A. baumanii in indicated drug concentrations (μg/mL).

FIG. 6 depicts an image of a multi-well plate showing a checkerboard assay result using novobiocin and compound 8 alone and in combination against A. baumanii in indicated drug concentrations (μg/mL).

FIG. 7 depicts pharmacokinetics of compound 3 in kidney, liver, spleen, lung, perioneal fluid, and plasma of male C57BL6 mice, following a subcutaneous administration at 10 mg/kg.

FIG. 8 depicts the survival rate of mice treated with different dosages of compound 3, following infection by S. aureus.

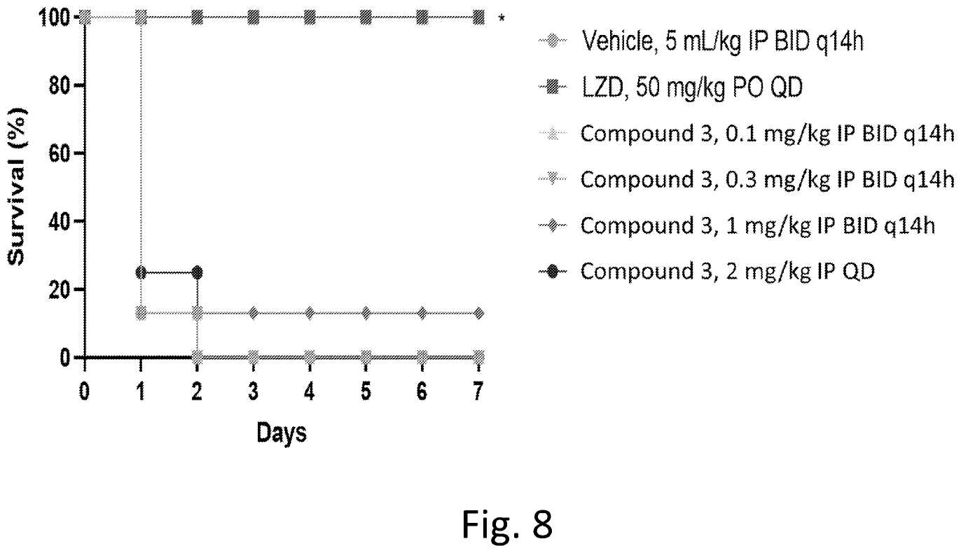

FIG. 9 depicts % cell viability following incubation with different concentrations of compound 3.

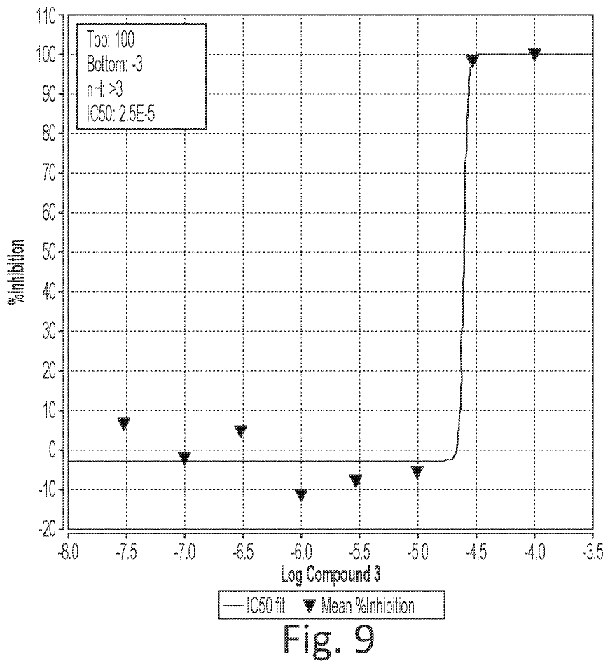

### BRIEF SUMMARY

The present disclosure provides a group of compounds (e.g., analogs of pentamidine disclosed herein) as shown in Formulae (I′), (I), (II), (I′-a), (I′-b), and (I′-c)) or stereoisomers or pharmaceutically acceptable salts thereof, which may be useful for treating a bacterial or fungal infection, or cancer. The bacterial infection may include those infections caused by gram negative or gram positive bacteria. Compositions, methods of synthesizing the same and methods for treating bacterial or fungal infection are disclosed herein. The present disclosure also provides a pharmaceutical formulation comprising at least one of the compounds with a pharmaceutically acceptable carrier, diluent or excipient therefor. The present invention is based on a discovery that the analogs of pentamidine are useful for treating bacterial or fungal infections. In some embodiments, the analogs of pentamidine are used as an adjuvant of an antibacterial agent commonly used to treat gram negative or gram positive bacterial infections.

In some embodiments, the compounds disclosed herein exhibit increased levels of potency and synergy with antibiotics against gram negative bacterial strains (e.g., rifampicin and novobiocin), particularly in their ability to inhibit growth of clinically relevant gram negative bacterial strains as compared to that of pentamidine. Non-limiting examples of the gram negative bacterial infections to which the present invention can be applied include infections caused by Serratia marcescens; Salmonella typhimurium, Salmonella choleraesuis; Acinetobacter baumannii; Citrobacter freundii; Pseudomonas aeruginosa; Escherichia coli; Stenotrophomonas maltophilia; Enterobacter cloacae; Enterobacter aerogenes; Staphylococcus aureus; Mycobacterium tuberculosis; Mycobacterium leprae; Mycobacterium avium complex; Neisseria meningitidis; and Klebsiella pneumoniae. When combined antibacterial drugs such as novobiocin and rifampicin, these amidine compounds effectively demonstrate increased growth inhibition or cytotoxicity against gram negative bacterial strains as compared to when the antibiotics were used alone.

In some embodiments, these properties of the compounds disclosed herein are highly desirable as adjuvant therapy in a treatment against gram negative infections, particularly the ones characterized as a “difficult-to-treat” in humans, such as multidrug-resistant gram negative bacterial infections (e.g., MRSA) that are often prescribed with novobiocin or rifampicin in hospital settings.

In some embodiments, the compounds disclosed herein exhibit increased levels of potency and antibiotic activity against gram positive bacterial strains as compared to pentamidine.

In some embodiments, the compounds disclosed herein exhibit antifungal activity. Non-limiting examples of the fungal infections to which the present invention can be applied includes infections caused by Candida parapsilosis, Candida krusei, Paecilomyces variotii, Candida albicans, Aspergillus fumigatus, Blastomyces dermatitidis, Candida auris, Candida glabrata, Candida guilliermondii, and Cryptococcus neoformans.

### DETAILED DESCRIPTION

Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition. The use of the term “including,” as well as other forms of the term, such as “includes” and “included,” is not limiting.

As used herein, “a” or “an” means “at least one” or “one or more.”

As used herein, “or” means “and/or.”

As used herein, the term “alkyl” refers to saturated hydrocarbon groups in a straight, branched, or cyclic configuration or any combination thereof, and particularly contemplated alkyl groups include those having ten or less carbon atoms, especially 1-6 carbon atoms and lower alkyl groups having 1-4 carbon atoms. Exemplary alkyl groups are methyl, ethyl, propyl, isopropyl, butyl, sec-butyl, tert-butyl, pentyl, isopentyl, hexyl, cyclopropylmethyl, and the like. Alkyl groups can be unsubstituted, or they can be substituted to the extent that such substitution is chemically feasible. Typical substituents include, but are not limited to, halo, ═O, ═N—CN, ═N—OR^{a}, ═NR^{a}, —OR^{a}, —NR^{a}₂, —SR^{a}, —SO₂R^{a}, —SO₂NR^{a}₂, —NR^{a}SO₂R^{a}, —NR^{a}CONR^{a}₂, —NR^{a}COOR^{a}, —NR^{a}COR^{a}, —NO₂, —CN, —COOR^{a}, —CONR^{a}₂, —OOCR^{a}, —COR^{a}, and —R^{a}, wherein each R^{a} is independently H, C₁-C₈ alkyl, C₂-C₈ heteroalkyl, C₃-C₈ heterocyclyl, C₄-C₁₀ heterocycloalkyl, C₁-C₈ acyl, C₂-C₈ heteroacyl, C₂-C₈ alkenyl, C₂-C₈ heteroalkenyl, C₂-C₈ alkynyl, C₂-C₈ heteroalkynyl, C₆-C₁₀ aryl, or C₅-C₁₀ heteroaryl, and each R^{a} is optionally substituted with halo, ═O, ═N—CN, ═N—OR^{b}, ═NR^{b}, —OR^{b}, —NR^{b}₂, —SR^{b}, —SO₂R^{b}, —SO₂NR^{b}₂, —NR^{b}SO₂R^{b}, —NR^{b}CONR^{b}₂, —NR^{b}COOR^{b}, —NR^{b}COR^{b}, —NO₂, —CN, —COOR^{b}, —CONR^{b}₂, —OOCR^{b}, —COR^{b}, and —R^{b}, wherein each R^{b} is independently H, C₁-C₈ alkyl, C₂-C₈ heteroalkyl, C₃-C₈ heterocyclyl, C₄-C₁₀ heterocycloalkyl, C₁-C₈ acyl, C₂-C₈ heteroacyl, C₂-C₈ alkenyl, C₂-C₈ heteroalkenyl, C₂-C₈ alkynyl, C₂-C₈ heteroalkynyl, C₆-C₁₀ aryl, or C₅-C₁₀ heteroaryl. Alkyl, alkenyl and alkynyl groups can also be substituted by C₁-C₈ acyl, C₂-C₈ heteroacyl, C₆-C₁₀ aryl or C₅-C₁₀ heteroaryl, each of which can be substituted by the substituents that are appropriate for the particular group. Where a substituent group contains two R^{a} or R^{b} groups on the same or adjacent atoms (e.g., —NR^{b}₂, or —NR^{b} —C(O)R^{b}), the two R^{a} or R^{b} groups can optionally be taken together with the atoms in the substituent group to which are attached to form a ring having 5-8 ring members, which can be substituted as allowed for the R^{a} or R^{b} itself, and can contain an additional heteroatom (N, O or S) as a ring member.

As used herein, the term “alkenyl” refers to hydrocarbon chain having at least two carbon atoms and at least one carbon-carbon double bond and includes straight, branched, or cyclic alkenyl groups having two to ten carbon atoms. Non-limiting examples of “alkenyl” include ethenyl, propenyl, butenyl, pentenyl, and cyclic alkenyl groups. An alkenyl can be unsubstituted or substituted with one or more suitable substituents.

As used herein, the term “alkynyl” refers to unbranched and branched hydrocarbon moieties having at least two (preferably three) carbon atoms and at least one carbon-carbon triple bond and includes ethynyl, propynyl, butynyl, cyclopropylethynyl, and the like. An alkynyl can be unsubstituted or substituted with one or more suitable substituents.

As used herein, the term “alkoxy” refers to the alkyl groups above bound through oxygen, examples of which include methoxy, ethoxy, propyloxy, isopropoxy, tert-butoxy, methoxyethoxy, benzyloxy, allyloxy, and the like. In addition, alkoxy also refers to polyethers such as —O—(CH₂)₂—O—CH₃, and the like. An alkoxy can be any hydrocarbon group connected through an oxygen atom wherein the hydrocarbon portion may have any number of carbon atoms, typically 1-10 carbon atoms, may further include a double or triple bond and may include one or two oxygen, sulfur or nitrogen atoms in the alkyl chains. An alkoxy can be unsubstituted or substituted with one or more suitable substituents, e.g., aryl, heteroaryl, cycloalkyl, and/or heterocyclyl.

As used herein, the term “cycloalkyl” refers to cyclic alkane in which a chain of carbon atoms of a hydrocarbon forms a ring, and includes a monocyclic or polycyclic hydrocarbon ring group, for example, cyclopropyl, cyclobutyl, cyclopentyl, cyclohexyl, cycloheptyl, cyclooctyl, cyclodecyl, adamantyl, norpinanyl, decalinyl, norbornyl, housanyl, and the like. Further, a cycloalkyl can also include one or two double bonds, which form the “cycloalkenyl” groups (e.g., cyclopropenyl, cyclobutenyl, cyclopentenyl, cyclohexenyl, cycloheptenyl, cyclooctenyl, norbornenyl, norbornadienyl, and the like). A cycloalkyl can also comprise one or more heteroatoms and referred to as “cycloheteroalkyl” and can include, for example, piperazinyl piperidinyl, morpholinyl, thiomorpholinyl, oxanyl, dioxanyl (e.g., 1,4-dioxanyl), thianyl, dithianyl, hexahydro-1,3,5-triazinyl, trioxanyl, trithianyl, pyrrolidinyl, imidazolidinyl, pyranyl, tetrahydropyranyl, pyrazolidinyl, oxolanyl, oxazolidinyl, thiolanyl, thiazolidinyl, pyrrolinyl, pyrazolinyl, imidazolinyl, tetrahydrofuranyl, and the like. A cycloalkyl or cycloheteroalkyl group can be unsubstituted or substituted with one or more suitable substituents.

As used herein, the term “amidine” or “Am” refers to a group of —CNH₂NH as shown in the following structure:

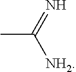

As used herein, the term “hetero” refers to an atom of any element other than carbon or hydrogen. As used herein, the term “heteroatom” means nitrogen (N), oxygen (O), or sulfur (S).

As used herein, the term “heterocycle” or “heterocyclyl” encompasses all limitations of “cycloheteroalkyl” and “heteroaryl” groups in so far as chemically feasible. The term “heterocycle” or “heterocyclyl” refers to any compound in which a plurality of atoms forms a ring via a plurality of covalent bonds, wherein the ring includes at least one atom other than a carbon atom as a ring member. A heterocycle can be saturated, unsaturated, or partially unsaturated. An unsaturated heterocycle can be aromatic aryl. Non-limiting examples of a heterocyclic ring include 3-, 4-, 5-, 6-, 7-, 8- and 9-membered monocyclic rings containing one or more N, O, or S as the non-carbon member(s) and are as follows: (1) a saturated 3 atom heterocyclic ring can be, for example, aziridinyl, diaziridinyl, oxiranyl, dioxiranyl, oxaziridinyl, thiiranyl, or the like, and an unsaturated 3 atom heterocyclic ring can be, for example, azirinyl, oxirenyl, thiirenyl, diazirinyl, or the like; (2) a saturated 4 atom heterocyclic ring can be, for example, azetidinyl, diazetidinyl, oxetanyl, dioxetanyl, thietanyl, dithietanyl, or the like, and an unsaturated 4 atom heterocyclic ring can be, for example, azetyl, diazetyl, oxetyl, dioxetyl, thietyl, dithietyl, or the like; (3) a saturated 5 atom heterocyclic ring can be, for example, pyrrolidinyl, imidazolidinyl, pyrazolidinyl, oxolanyl, oxazolidinyl, thiolanyl, thiazolidinyl, or the like, and an unsaturated and partially unsaturated 5 atom heterocyclic ring can be, for example, pyrrolyl, pyrrolinyl, pyrazolyl, pyrazolinyl, imidazolyl, imidazolinyl, triazolyl, tetrazolyl, thiophenyl, thiazolyl, dithiazolyl, thiazolinyl, isothiazolyl, thiadiazolyl, furanyl, furazanyl, oxazolyl, isoxazolyl, oxadiazolyl, or the like; (4) a saturated 6 atom heterocyclic ring can be, for example, piperidinyl, piperazinyl, morpholinyl, thiomorpholinyl, oxanyl, dioxanyl (e.g., 1,4-dioxacyclohexane), thianyl, dithianyl, hexahydro-1,3,5-triazinyl, trioxanyl, trithianyl, or the like, and an unsaturated 6 atom heterocyclic ring can be, for example, pyridinyl, diazinyl (e.g., pyrimidinyl, or pyridazinyl), pyranyl, oxazinyl (e.g., 1,2-oxazinyl; 1,3-oxazinyl, or 1,4-oxazinyl), thiazinyl, 1,4-dioxinyl, dithiinyl, triazinyl (e.g., 1,2,3-triazinyl, 1,2,4-triazinyl, or 1,3,5-triazinyl), tetrazinyl, pentazinyl, thiopyranyl, or the like; (5) a saturated 7 atom heterocyclic ring can be, for example, azepanyl, diazepanyl, oxepanyl, thiepanyl, or the like, and an unsaturated 7 atom heterocyclic ring can be, for example, azepinyl, diazepinyl, oxepinyl, thiepinyl, thiazepinyl, or the like; (6) a saturated 8 atom heterocyclic ring can be, for example, azocanyl, oxocanyl, thiocanyl, or the like, and an unsaturated 8 atom heterocyclic ring can be, for example, azocinyl, oxocinyl, thiocinyl, or the like; and (7) a saturated 9 atom heterocyclic ring can be, for example, azonanyl, oxonanyl, thionanyl, or the like, and an unsaturated 9 atom heterocyclic ring can be, for example, azoninyl, oxoninyl, thioninyl, or the like. Further contemplated heterocycles may be fused, for example, covalently bound with two atoms on the first non-heterocyclic group (e.g., phenyl) to one or two heterocycles (e.g., 1,4-dioxanyl, 1,4-dioxinyl, and tetrahydropyranyl), or covalently bound with two atoms on the first heterocyclic ring (e.g., pyrrolyl, imidazolyl, thiazolyl, pyrimidinyl, and pyridinyl) to one or two nonheterocyclic or heterocyclic group (e.g.,1,4-dioxanyl, 1,4-dioxinyl, and morpholinyl), and taken together are thus termed “fused heterocycle” or “fused heterocyclic moieties” or “heteroaryl-fused-cycloheteroalkyl” as used herein. The fused heterocycle can be, for example, a saturated or unsaturated (e.g., aromatic) bicyclic or tricyclic compound. Non-limiting examples of fused heterocycle include dihydrobenzodioxinyl, dihydrodioxinopyridinyl, dihydrodioxinopyridazinyl, dihydrodioxinopyrimidinyl, dihydrodioxinopyrazinyl, dihydropyrrolopyridinyl, tetrahydronaphthyridinyl, tetrahydropyridopyridazinyl, tetrahydropyridopyrazinyl, tetrahydropyridopyrimidinyl, chromanyl, indolyl, purinyl, isoindolyl, quinolinyl, isoquinolinyl, quinoxalinyl, quinazolinyl, quinolizinyl, 1,8-naphthyridinyl, pyrido[3,2-d]pyrimidinyl, pyrido[4,3-d]pyrimidinyl, pyrido[3,4-b]pyrazinyl, pyrido[2,3-b]pyrazinyl, pteridinyl, acridinyl, cinnolinyl, phthalazinyl, benzimidazolyl, phenazinyl, phenoxazinyl, phenothiazinyl, phenoxathiinyl, benzazepinyl, benzodiazepinyl, benzofuranyl, dibenzofuranyl, isobenzofuranyl, benzothiophenyl, benzoxazinyl, quinolin-2(1H)-onyl, isoquinolin-1(2H)-onyl, indazolyl, benzoxazolyl, benzisoxazolyl, benzothiazolyl, dibenzazepinyl, dibenzoxepinyl, dibenzothiazepinyl, dibenzothiepinyl, carbazolyl, fluorenyl, and the like. Where the heterocyclic ring is aromatic, it can be also referred to herein as “heteroaryl” or “heteroaromatic” as described further below. A heterocyclic ring that is not aromatic can be substituted with any group suitable for alkyl group substituents described above.

As used herein, the term “aryl” refers to unsubstituted or substituted aromatic monocyclic or polycyclic groups, which may further include one or more non-carbon atoms. The term “aryl” also includes aromatic rings fused to non-aromatic carbocyclic ring, or to a heterocyclyl group having 1-7 heteroatoms. The term “aryl” may be interchangeably used with “aryl ring,” “aromatic group,” and “aromatic ring.” An aryl group may contain 1-9 heteroatom(s) that are generally referred to as “heteroaryl.” Heteroaryl groups typically have 4 to 14 atoms, 1 to 9 of which are independently selected from the group consisting of N, O, and S. In a 5-8 membered aromatic group, for example, a heteroaryl group can contain 1-4 heteroatoms. An aryl or heteroaryl can be unsubstituted or substituted with one or more suitable substituents.

An aryl or heteroaryl can be a mono- or polycyclic (e.g., bicyclic) aromatic group. Typical aryl groups include, for example, phenyl and naphthalenyl and the like. Typical heteroaryl groups include, for example, quinolinyl, pyrrolyl, pyrazolyl, imidazolyl, triazolyl, tetrazolyl, thiophenyl, thiazolyl, dithiazolyl, thiazolinyl, isothiazolyl, thiadiazolyl, furanyl, furazanyl, oxazolyl, isoxazolyl, oxadiazolyl, pyridinyl, diazinyl (e.g., pyrazinyl, pyrimidinyl, or pyridazinyl), triazinyl (e.g., 1,2,3-triazinyl, 1,2,4-triazinyl, or 1,3,5-triazinyl), pyranyl, oxazinyl (e.g., 1,2-oxazinyl; 1,3-oxazinyl, or 1,4-oxazinyl), thiazinyl, dioxinyl, dithiinyl, triazinyl, tetrazinyl, pentazinyl, thiopyranyl, azepinyl, diazepinyl, oxepinyl, thiepinyl, thiazepinyl, azocinyl, oxocinyl, thiocinyl, azoninyl, oxoninyl, thioninyl, indolyl, indazolyl, purinyl, isoindolyl, quinolinyl, isoquinolinyl, quinoxalinyl, acridinyl, quinazolinyl, cinnolinyl, phthalazinyl, benzimidazolyl, benzofuranyl, isobenzofuranyl, benzoxazolyl, benzisoxazolyl, benzothiazolyl, or the like. Polycyclic aryl or polycyclic heteroaryl groups can be formed by fusing (i.e., covalently bonding) 2 atoms on the first aryl or heteroaryl ring with at least one carbocyclic or heterocyclic group, and are thus termed “fused aryl” or “heteroaryl-fused-cycloheteroalkyl.”

As used herein, the term “heteroaryl-fused-cycloheteroalkyl” refers to a heterocyclyl moiety consisting of a monocyclic heteroaryl group, such as pyridinyl or furanyl, fused to a cycloheteroalkyl group, in which the heteroaryl and cycloheteroalkyl pails are as defined herein. Exemplary heteroaryl-fused-heterocycloalkyl groups include dihydrodioxinopyridinyl, dihydrodioxinopyridazinyl, dihydrodioxinopyrimidinyl, dihydrodioxinopyrazinyl, dihydrodioxinotriazinyl, dihydropyrrolopyridinyl, dihydrofuranopyridinyl and dioxolopyridinyl. The heteroaryl-fused-heterocycioalkyl group may be attached to the remainder of the molecule by any available carbon or nitrogen atom.

Typical heteroaryl groups include 5 or 6 member monocyclic aromatic groups such as pyridinyl, pyrimidyl, pyrazinyl, pyridazinyl, thienyl, furanyl, pyrrolyl, pyrazolyl, thiazolyl, oxazolyl, isothiazolyl, isoxazolyl, thiophenyl, triazolyl (1,2,4-triazolyl and 1,2,3-triazolyl), tetrazolyl, furazanyl, oxadiazolyl (1,2,5-oxadiazolyl and 1,2,3-oxadiazolyl), and imidazolyl and the fused bicyclic moieties formed by fusing one of heterocyclic groups with a phenyl ring or with any of the heteroaromatic monocyclic groups include indolyl, benzimidazolyl, indazolyl, benzotriazolyl, isoquinolyl, quinolyl, benzothiazolyl, benzofuranyl, pyrazolopyridinyl, pyrazolopyrimidyl, quinazolinyl, quinoxalinyl, cinnolinyl, imidazopyrimidinyl, and the like.

As used herein, the term “monocyclic” refers to an unsubstituted or substituted single ring structure. As used herein, the terms “polycyclic” and “bicyclic” refer to an unsubstituted or substituted poly-ring structure that comprises at least two ring structures fused by any two adjacent atoms. A bicyclic ring can be an aryl or heteroaryl ring fused to an aromatic ring or a non-aromatic carbocyclic ring such as cycloalkyl or cycloheteroalkyl. A bicyclic ring can be also non-aromatic carbocyclic ring fused to another non-aromatic carbocyclic ring such as cycloalkyl or cycloheteroalkyl. Non-limiting examples of bicyclic rings include dihydrobenzodioxinyl, dihydrodioxinopyridinyl, dihydrodioxinopyridazinyl, dihydrodioxinopyrimidinyl, dihydrodioxinopyrazinyl, dihydropyrrolopyridinyl, tetrahydronaphthyridinyl, tetrahydropyridopyridazinyl, tetrahydropyridopyrazinyl, tetrahydropyridopyrimidinyl, chromanyl, decalinyl, purinyl, indolyl, isoindolyl, quinolyl, quinazolinyl, benzimidazolyl, imidazopyridinyl, cinnolinyl, phthalazinyl, imidazopyrimidinyl, and the like. Any monocyclic or fused bicyclic system which has the characteristics of aromaticity in terms of electron distribution throughout the ring system is included in this definition. It also includes bicyclic groups where at least the ring which is directly attached to the remainder of the molecule has the characteristics of aromaticity.

Aryl and heteroaryl groups can be substituted where permitted. Suitable substituents include, but are not limited to, halo, R^{a}, —OR^{a}, —NR^{a}₂, —SR^{a}, —SO₂R^{a}, —SO₂NR^{a}₂, —NR^{a}SO₂R^{a}, —NR^{a}CONR^{a}₂, —NR^{a}COOR^{a}, —NR^{a}COR^{a}, —CN, —COOR^{a}, —CONR^{a}₂, —OOCR^{a}, —COR^{a}, and —NO₂, wherein each R^{a} is independently H, C₁-C₈ alkyl, C₂-C₈ heteroalkyl, C₃-C₈ heterocyclyl, C₄-C₁₀ heterocycloalkyl, C₁-C₈ acyl, C₂-C₈ heteroacyl, C₂-C₈ alkenyl, C₂-C₈ heteroalkenyl, C₂-C₈ alkynyl, C₂-C₈ heteroalkynyl, C₆-C₁₀ aryl, or C₅-C₁₀ heteroaryl, and each R^{a} is optionally substituted with halo, ═O, ═N—CN, ═N—OR^{b}, ═NR^{b}, —OR^{b}, —NR^{b}₂, —SR^{b}, —SO₂R^{b}, —SO₂NR^{b}₂, —NR^{b}SO₂R^{b}, —NR^{b}CONR^{b}₂, —NR^{b}COOR^{b}, —NR^{b}COR^{b}, —CN, —COOR^{b}, —CONR^{b}₂, —OOCR^{b}, —COR^{b}, and —NO₂, wherein each R^{b} is independently H, C₁-C₈ alkyl, C₂-C₈ heteroalkyl, C₃-C₈ heterocyclyl, C₄-C₁₀ heterocycloalkyl, C₁-C₈ acyl, C₂-C₈ heteroacyl, C₂-C₈ alkenyl, C₂-C₈ heteroalkenyl, C₂-C₈ alkynyl, C₂-C₈ heteroalkynyl, C₆-C₁₀ aryl, or C₅-C₁₀ heteroaryl. Alkyl, alkenyl and alkynyl groups can also be substituted by C₁-C₈ acyl, C₂-C₈ heteroacyl, C₆-C₁₀ aryl or C₅-C₁₀ heteroaryl, each of which can be substituted by the substituents that are appropriate for the particular group. Where a substituent group contains two R^{a} or R^{b} groups on the same or adjacent atoms (e.g., —NR^{b}₂, or —NR^{b}—C(O)R^{b}), the two R^{a} or R^{b} groups can optionally be taken together with the atoms in the substituent group to which are attached to form a ring having ₅-₈ ring members, which can be substituted as allowed for the R^{a} or R^{b} itself, and can contain an additional heteroatom (N, O or S) as a ring member.

The term “sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cycloalkenyl, SO₂-aryl, SO₂-substituted aryl, SO₂-heteroaryl, SO₂-substituted heteroaryl, SO₂-heterocyclic, and SO₂-substituted heterocyclic, wherein each alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

As used herein, the term “acyl” when used without the “substituted” modifier refers to the group —C(O)R, in which R is a hydrogen, alkyl, aryl, halides, aralkyl or heteroaryl, as those terms are defined herein.

As used herein, the term “acyloxy” refers a straight-chain or branched alkanoyl group having 1 to 6 carbon atoms, such as formyl, acetyl, propanoyl, butyryl, valeryl, pivaloyl and hexanoyl, and arylcarbonyl group described below, or a heteroarylcarbonyl group described below. The aryl moiety of the arylcarbonyl group means a group having 6 to 16 carbon atoms such as phenyl, biphenyl, naphthyl, or pyrenyl. The heteroaryl moiety of the heteroarylcarbonyl group contains at least one hetero atom from O, N, and S, such as pyridinyl, pyrimidyl, pyrroleyl, furyl, benzofuryl, thienyl, benzothienyl, imidazolyl, triazolyl, quinolyl, iso-quinolyl, benzoimidazolyl, thiazolyl, benzothiazolyl, oxazolyl, and indolyl.

As used herein, the term “carboxylic acid” refers to a group —C(O)OH.

As used herein, the term “ester,” as used herein, refers to a group —C(O)O—.

As used herein, the term “nitro” means —NO₂.

As used herein, the term “cyano” means —CN.

As used herein, the term “azido” means relating to a monovalent group containing —N₃.

As used herein, the term “sulfhydryl” means thiol, —SH.

As used herein, the term “amine” means primary, secondary and tertiary amines, —R—NH₂, —R—NH—R′, and —R—N—(R″)R′, respectively.

As used herein, the term “amide” means primary, secondary and tertiary amides, —R—C(O)NH₂, —R—C(O)NH—R′, and —R—C(O)NR′R″, respectively.

As used herein, the term “carbonate” means ester of carbonic acid, a group containing C(═O)(O—)₂.

As used herein, the term “carbamate” means a group containing NH₂COOH.

As used herein, the term “hydroxyl” means —OH.

As used herein, the terms “halo,” “halogen,” and “halide” mean fluoro (—F), chloro (—Cl), bromo (—Br), and iodo (—I).

As used herein, the term “haloalkyl” refers to any alkyl having one or more hydrogen atoms replaced by one or more halogen atoms. Non-limiting examples of haloalkyl include —CF₃, —CFH₂, —CF₂H, and the like.

As used herein, the term “arylalkyl” refers to any alkyl in which one or more hydrogen atoms are replaced by an aryl or heteroaryl group. Examples of arylalkyl include benzyl (C₆H₅CH₂—) and the like.

As used herein, the term “hydroxyalkyl” refers to any hydroxy derivative of alkyl and includes any alkyl having one or more hydrogen atoms replaced by a —OH group.

The term “haloalkyl” refers to an alkyl group as described above with one or more hydrogen atoms on the alkyl group substituted with a halo group. Examples of such groups include, without limitation, fluoroalkyl groups, such as fluoroethyl, difluoromethyl, trifluoromethyl, trifluoroethyl and the like.

The term “haloalkoxy” refers to the group alkyl—O— with one or more hydrogen atoms on the alkyl group substituted with a halo group (e.g., —F, —Cl, —Br, and —I) and include, for example, groups such as trifluoromethoxy and the like.

The term “substituted” as used herein refers to a replacement of a hydrogen atom of the unsubstituted group with a functional group, and particularly contemplated functional groups include nucleophilic groups (e.g., —NH₂, —OH, —SH, —CN, etc.), electrophilic groups (e.g., C(O)OR, C(X)OH, etc.), polar groups (e.g., —OH), non-polar groups (e.g., heterocycle, aryl, alkyl, alkenyl, alkynyl, etc.), ionic groups (e.g., —NH₃⁺), and halogens (e.g., —F, —Cl), NHCOR, NHCONH₂, OCH₂COOH, OCH₂CONH₂, OCH₂CONHR, NHCH₂COOH, NHCH₂CONH₂, NHSO₂R, OCH₂-heterocycles, POSH, SO₃H, amino acids, and all chemically reasonable combinations thereof. Moreover, the term “substituted” also includes multiple degrees of substitution, and where multiple substituents are disclosed or claimed, the substituted compound can be independently substituted by one or more of the disclosed or claimed substituent moieties.

Unless indicated otherwise, the nomenclature of substituents that are not explicitly defined herein are arrived at by naming the terminal portion of the functionality followed by the adjacent functionality toward the point of attachment. For example, the substituent “alkylaryloxycarbonyl” refers to the group (alkyl)-(aryl)—O—C(O)—.

As to any of the groups disclosed herein which contain one or more substituents, it is understood that such groups do not contain any substitution or substitution patterns which are sterically impractical and/or synthetically non-feasible. In addition, the subject compounds include all stereochemical isomers arising from the substitution of these compounds. As used herein, the term “stereoisomers” refers to compounds which have identical chemical constitution, but differ with regard to the arrangement of the atoms or groups in space. In addition to the disclosure herein, in a certain embodiment, a group that is substituted has 1 substituent, 1 or 2 substituents, 1, 2, or 3 substituents, or 1, 2, 3, or 4 substituents.

As used herein, the term “administration” or “administering” of the subject compound refers to providing a compound of the invention to a subject in need of treatment.

As used herein, the term “acceptable” with respect to a formulation, composition or ingredient, as used herein, means having no persistent detrimental effect on the general health of the subject being treated.

The term “about” when referring to a number or a numerical range means that the number or numerical range referred to is an approximation within experimental variability (or within statistical experimental error), and thus the number or numerical range may vary from, for example, between 1% and 10% of the stated number or numerical range.

As used herein, the term “carrier” refers to chemical compounds or agents that facilitate the incorporation of a compound described herein into cells or tissues.

As used herein, the terms “comprise,” “have,” and “include” are open-ended linking verbs. Any forms or tenses of one or more of these verbs, such as “comprises,” “comprising,” “has,” “having,” “includes,” and “including” are open-ended. For example, any method that “comprises,” “has,” or “includes” one or more moieties is not limited to possessing only those one or more moieties and also covers other unlisted moieties.

As used herein, “treatment” or “treating” is an approach for obtaining beneficial or desired results including clinical results. For purposes of this disclosure, beneficial or desired results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, delaying the occurrence or recurrence of the disease, delay or slowing the progression of the disease, ameliorating the disease state, providing a remission (whether partial or total) of the disease, decreasing the dose of one or more other medications required to treat the disease, enhancing effect of another medication, delaying the progression of the disease, increasing the quality of life, and/or prolonging survival. The methods of the present disclosure contemplate any one or more of these aspects of treatment.

A “pharmaceutically acceptable salt” is a salt formed from an acid and a basic group of pentamidine analogs. Examples of such salts include acid addition salts and base addition salts, such as inorganic acid salts or organic acid salts (e.g., hydrochloric acid salt, dihydrochloric acid salt, sulfuric acid salt, citrate, hydrobromic acid salt, hydroiodic acid salt, nitric acid salt, bisulfate, phosphoric acid salt, super phosphoric acid salt, isonicotinic acid salt, acetic acid salt, lactic acid salt, salicylic acid salt, tartaric acid salt, pantothenic acid salt, ascorbic acid salt, succinic acid salt, maleic acid salt, fumaric acid salt, gluconic acid salt, saccharinic acid salt, formic acid salt, benzoic acid salt, glutaminic acid salt, methanesulfonic acid salt, ethanesulfonic acid salt, benzenesulfonic acid salt, p-toluenesulfonic acid salt, pamoic acid salt (pamoate)), as well as salts of aluminum, calcium, lithium, magnesium, calcium, sodium, zinc, and diethanolamine. It is to be understood that reference to a pentamidine analog, or a stereoisomer or pharmaceutically acceptable salt thereof, includes pharmaceutically acceptable salts of compound disclosed herein. Examples of such pharmaceutically acceptable salts include, but are not limited to, isethionate, gluconate, and mesylate.

As used herein, the term “hydrogen” refers to a hydrogen atom (—H) and deuterium (heavy hydrogen, non-radioactive isotope of hydrogen, D or ₂H). It is to be understood that the present invention contemplates deuterated compound versions of all molecules of the present disclosure which can be synthesized by converting a hydrogen atom to ₂H at a place where a hydrogen atom is present.

### Pentamidine Analogs

The present invention is drawn to compounds of Formulae (I′) (I), (II), (I′-a), (I′-b), or (I′-c), or a stereoisomer or pharmaceutically acceptable salt thereof.

In some embodiments, the compound is a compound of Formula (I′),

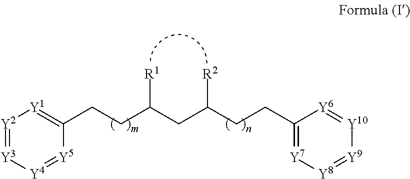

or a stereoisomer or pharmaceutically acceptable salt thereof, wherein:

m and n are independently 0, 1, 2 or 3;

R¹ and R² are independently hydrogen or halo, or R¹ taken together with R² forms a saturated, unsaturated or partially unsaturated 3-9 member ring; and

Y¹ through Y¹⁰ are independently CR³, wherein R³ is independently hydrogen, heterocycle, or amidine, or R³ taken together with another R³ at an immediately adjacent carbon atom forms

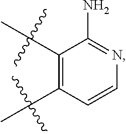 [SMILES: *c1ccnc(N)c1*]

wherein:
R³ at Y¹, Y⁵, Y⁶ and Y⁷ is hydrogen, and
R³ at one of Y², Y³ or Y⁴ and optionally at one of Y⁸, Y⁹, or Y¹⁰ is amidine, or taken together with another R³ at an immediately adjacent carbon atom forms

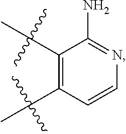 [SMILES: *c1ccnc(N)c1*]

provided that

when (1) R¹ taken together with R² forms the saturated, unsaturated or partially unsaturated 3-9 member ring, (2) R³ is amidine at one of Y², Y³, or Y⁴, and (3) R³ is amidine at one of Y⁸, Y⁹, or Y¹⁰, then m and n are independently 1, 2 or 3; and

when (1) R¹ and R² are both hydrogen, (2) R³ is amidine at one of Y², Y³, or Y⁴, and (3) R³ is amidine at one of Y⁸, Y⁹, or Y¹⁰, then R³ at Y³ and R³ at Y⁹ are independently hydrogen or heterocycle.

In some embodiments, when R¹ and R² are hydrogen, R³ is amidine at one of Y², Y³, or Y⁴, and R³ is amidine at one of Y⁸, Y⁹, or Y¹⁰, then R³ at Y³ and R³ at Y⁹ are independently hydrogen or heterocycle. In some embodiments, when amidine (is not present at any one of Y⁸, Y₉ and Y¹⁰, R³ can be a heterocycle at only one of Y⁸, Y⁹, or Y¹⁰. In some embodiments, when R³ at Y², Y³, Y⁴, Y⁸, Y⁹, or Y¹⁰ is neither amidine nor the 3-9 member cyclic group, R³ is hydrogen as a default.

According to the present invention, m or n can be an integer of 0, 1, 2 or 3. In one embodiment, m is 1, and n is 1. In another embodiment, m is 0, and n is 0. In yet another embodiment, m is 1, and n is 0. In yet another embodiment, m is 0, and n is 1. In yet another embodiment, m is 1, and n is 2. In yet another embodiment, m is 2, and n is 1. In one particular embodiment, m is 0, and n is 0.

In some aspects, R¹ and R² are independently hydrogen (R¹=H; and R²=H).

In some other aspects, R¹ taken together with R² forms a saturated, unsaturated or partially unsaturated 3-9 member cyclic group. For example, R¹ taken together with R² forms 5 member saturated heterocycloalkyl or cycloalkyl. In another example, R¹ taken together with R² forms 6 member saturated heterocycloalky or cycloalkyl. In a specific example, R¹ taken together with R² forms cyclohexyl. In another example, R¹ taken together with R² forms 7 member saturated heterocycloalkyl or cycloalkyl.

In one aspect, R³ can be amidine at Y⁴ and Y⁸. For example, R¹ and R² are independently hydrogen, and R³ is amidine at Y⁴ and Y⁸. In another example, R¹ taken together with R² forms a saturated, unsaturated or partially unsaturated 3-9 member cyclic group as described above, and R³ is amidine at Y⁴ and Y⁸.

In another aspect, R³ can be amidine at Y² and Y¹⁰. In one embodiment, R¹ and R² are independently hydrogen, and R³ is amidine at Y² and Y¹⁰. In another embodiment, R¹ taken together with R² forms a saturated, unsaturated or partially unsaturated 3-9 member cyclic group, and R³ is amidine at Y² and Y¹⁰. In these embodiments, R¹ taken together with R² can form a saturated 5 member cycloalkyl. Further, R¹ taken together with R² can also form a saturated 6 member cycloalkyl (e.g., cyclohexyl). Lastly, R¹ taken together with R² can form a saturated 7 member cycloalkyl.

In yet another aspect, R³ can be amidine at Y³ and Y⁹. For example, R¹ and R² are independently hydrogen, and R³ is amidine at Y³ and Y⁹. In another example, R¹ taken together with R² forms a saturated, unsaturated or partially unsaturated 3-9 member cyclic group, and R³ is amidine at Y³ and Y⁹. In one embodiment, R¹ taken together with R² forms a saturated 5 member cycloalkyl. In another embodiment, R¹ taken together with R² forms a saturated 6 member cycloalkyl (e.g., cyclohexyl). In yet another embodiment, R¹ taken together with R² forms a saturated 7 member cycloalkyl.

In some aspects, when amidine

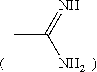

is not present at any one or Y⁸, Y⁹, or Y¹⁰, R³ can be a heterocycle at Y⁸, Y⁹, or Y¹⁰. When R₃ at Y², Y³, Y⁴, Y⁸, Y⁹, or Y¹⁰ is neither amidine nor the heterocycle, R³ is hydrogen as a default. For example, when R¹ and R² are independently hydrogen, R³ is amidine

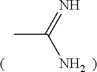

at Y² and a heterocycle (e.g., a saturated 5 member heterocycloalkyl) at Y¹⁰. In one embodiment, the heterocycle is a saturated 5 member heterocycloalkyl at Y¹⁰. Specifically, the saturated 5 member heterocycloalkyl at Y¹⁰ can be pyrrolidinyl. In another example, when R¹ and R² are independently hydrogen, R³ can be amidine at Y³ and a heterocycle at Y⁹ (e.g., saturated 5 member heterocycloalkyl). In one particular embodiment under this aspect, the heterocycle at Y⁹ is a saturated 5 member heterocycloalkyl at Y⁹ can be pyrrolidinyl. Further, when R¹ and R² independently hydrogen and R³ can be amidine at Y⁴ and a saturated 5 member heterocycloalkyl at Y⁸. Again, the saturated ₅ member heterocycloalkyl can be pyrrolidinyl as above. In another example, when R¹ taken together with R² forms a saturated, unsaturated or partially unsaturated 3-9 member cyclic group, R³ can be amidine at Y³ and a saturated 5 member heterocycloalkyl (e.g., pyrrolidinyl) at Y⁹. Further, in one preferred embodiment, the saturated heterocycloalkyl at Y⁹ is pyrrolidinyl. In one particular embodiment, R¹ taken together with R² forms a saturated 6 member cycloalkyl (e.g., cyclohexyl); and R³ is amidine at Y³ and pyrrolidinyl at Y⁹. In another particular embodiment, R¹ taken together with R² forms a saturated ₆ member cycloalkyl (e.g., cyclohexyl); and R³ is amidine at Y² and pyrrolidinyl at Y¹⁰. In another particular embodiment, R¹ taken together with R² forms a saturated ₆ member cycloalkyl (e.g., cyclohexyl); and R³ is amidine at Y⁴ and pyrrolidinyl at Y⁸.

In one variation of all formulae described in the present application, the carbons bearing R¹ and R² are both in the “S” configuration. In another variation, the carbons bearing R¹ and R² are both in the “R” configuration. In another variation, one of the carbons bearing R¹ and R² is in the “R” configuration while the other is in the “S” configuration. Mixtures of a compound of the formulae described herein are also embraced, including racemic or non-racemic mixtures of a given compound, and mixtures of two or more compounds of different chemical formulae.

In the descriptions herein, it is understood that every description, variation, embodiment or aspect of a moiety may be combined with every description, variation, embodiment or aspect of other moieties the same as if each and every combination of descriptions is specifically and individually listed.

In some embodiments, the compound is a compound of Formula (I),

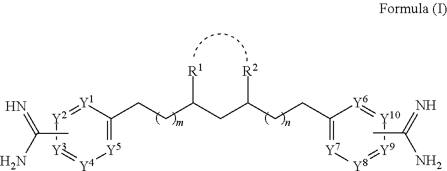

or a stereoisomer or pharmaceutically acceptable salt thereof, wherein:

m and n are independently 0, 1, 2 or 3;

R¹ and R² are independently hydrogen or halo, or R¹ taken together with R² forms a saturated, unsaturated or partially unsaturated 3-9 member ring; and

Y¹ through Y¹⁰ are independently CR³, wherein R³ is hydrogen at Y¹, Y⁵, Y⁶ and Y⁷ and R³ and Y², Y³, Y⁴, Y⁸, Y⁹, and Y¹⁰ is independently hydrogen, heterocycle, or absent when a corresponding carbon is attached to an amidine,

provided that
when R¹ taken together with R² forms the saturated, unsaturated or partially unsaturated 3-9 member ring, then m and n are independently 1, 2 or 3; and
when R¹ and R² are both hydrogen, then R³ at Y³ and R³ at Y⁹ are independently hydrogen or heterocycle.

In some embodiments, the compound is a compound of Formula (II),

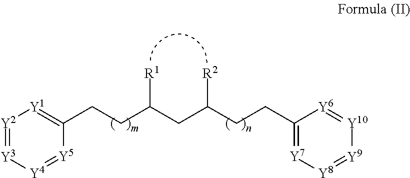

or a stereoisomer or pharmaceutically acceptable salt thereof, wherein:

m and n are independently 0, 1, 2 or 3;

R¹ and R² are independently hydrogen or halo, or R¹ taken together with R² forms a saturated, unsaturated or partially unsaturated 3-9 member ring; and

Y¹ through Y¹⁰ are independently CR³, wherein R³ is independently hydrogen, heterocycle, or amidine, wherein:
R³ at Y¹, Y⁵, Y⁶ and Y⁷ is hydrogen, and
R³ at one of Y², Y³ or Y⁴ and optionally at one of Y⁸, Y⁹, or Y¹⁰ is amidine,

provided that
when (1) R¹ taken together with R² forms the saturated, unsaturated or partially unsaturated ₃-₉ member ring, (2) R³ is amidine at one of Y², Y³, or Y⁴, and (3) R³ is amidine at one of Y⁸, Y⁹, or Y¹⁰, then m and n are independently 1, 2 or 3; and
when (1) R¹ and R² are both hydrogen, (2) R³ is amidine at one of Y², Y³, or Y⁴, and (3) R³ is amidine at one of Y⁸, Y⁹, or Y¹⁰, then R³ at Y³ and R³ at Y⁹ are independently hydrogen or heterocycle.

In some embodiments, the compound is a compound of Formula (I′-a),

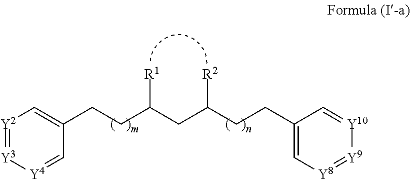

or a stereoisomer or pharmaceutically acceptable salt thereof, wherein:

m and n are independently 1, 2 or 3;

R¹ taken together with R² forms a saturated, unsaturated or partially unsaturated 3-9 member ring; and

Y² through Y⁴ and Y⁸ through Y¹⁰ are independently CR³, wherein R³ is independently hydrogen, heterocycle, or amidine, wherein:
R³ is amidine at one of Y², Y³ or Y⁴ and optionally at one of Y⁸, Y⁹, or Y¹⁰.

In some embodiments, the compound is a compound of Formula (I′-b),

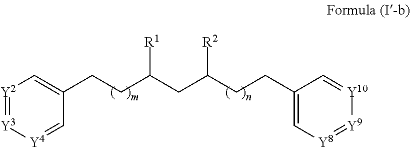

or a stereoisomer or pharmaceutically acceptable salt thereof, wherein:

m and n are independently 0, 1, 2 or 3;

R¹ and R² are independently hydrogen or halo; and

Y² through Y⁴ and Y⁸ through Y¹⁰ are independently CR³, wherein R³ is independently hydrogen, heterocycle, or amidine, wherein:
R³ is amidine at one of Y², Y³ or Y⁴ and optionally at one of Y⁸, Y⁹, or Y¹⁰;

provided that
when R³ is amidine at one of Y², Y³, or Y⁴, and R³ is amidine at one of Y⁸, Y⁹, or Y¹⁰, then R³ at Y³ and R³ at Y⁹ are independently hydrogen or heterocycle.

In some embodiments, the compound is a compound of Formula (I′-c),

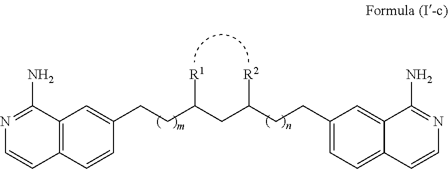

or a stereoisomer or pharmaceutically acceptable salt thereof, wherein:

m and n are independently 0, 1, 2 or 3;

R¹ and R² are independently hydrogen or halo, or R¹ taken together with R² forms a saturated, unsaturated or partially unsaturated 3-9 member ring.

In some embodiments, the compound is selected from the group consisting of compounds 1-8 in Table 1, or a stereoisomer or pharmaceutically acceptable salt thereof. In some embodiments, the compound is 3,3′-(heptane-1,7-diyl)dibenzimidamide, or a stereoisomer or pharmaceutically acceptable salt thereof. In some embodiments, the compound is 3-(7-(3-(pyrrolidin-3-yl)phenyl)heptyl)benzimidamide, or a stereoisomer or pharmaceutically acceptable salt thereof. In some embodiments, the compound is 4,4′-(2,2′-((1R,3S)-cyclohexane-1,3-diyl)bis(ethane-2,1-diyl))dibenzimidamide, or a stereoisomer or pharmaceutically acceptable salt thereof. In some embodiments, the compound is 4,4′-(2,2′-((1R,3R)-cyclohexane-1,3-diyl)bis(ethane-2,1-diyl))dibenzimidamide, or a stereoisomer or pharmaceutically acceptable salt thereof. In some embodiments, the compound is 4-(2-((1S,3R)-3-(4-(pyrrolidin-3-yl)phenethyl)cyclohexyl)ethyl)benzimidamide, or a stereoisomer or pharmaceutically acceptable salt thereof. In some embodiments, the compound is 3,3′-(((1R,3S)-cyclohexane-1,3-diyl)bis(ethane-2,1-diyl))dibenzimidamide, or a stereoisomer or pharmaceutically acceptable salt thereof. In some embodiments, the compound is 7,7′-(heptane-1,7-diyl)bis(isoquinolin-1-amine), or a stereoisomer or pharmaceutically acceptable salt thereof. In some embodiments, the compound is 4-(7-(4-(pyrrolidine-3-yl)phenyl)heptyl)benzimidamide, or a stereoisomer or pharmaceutically acceptable salt thereof.

### Methods

The compounds and compositions described herein can be administered to a mammalian subject (e.g., human patient) in need of treatment for bacterial or fungal infections alone or in combination with an additional therapeutic agent.

In some embodiments, the compounds and compositions described herein are administered to a mammalian subject (e.g., human patient) in need of treatment for a bacterial infection as adjuvant therapy of an antibiotic agent, including but not limited to, novobiocin and rifampicin and other antibiotics used to treat bacterial infections, for example, cephalosporins (e.g., ceftriaxone-cefotaxime, ceftazidime, and others), fluoroquinolones (e.g., ciprofloxacin, levofloxacin and others), aminoglycosides (e.g., gentamicin, amikacin and others), imipenem, broad-spectrum penicillins with or without β-lactamase inhibitors (e.g., amoxicillin-clavulanic acid, piperacillin-tazobactam and others), trimethoprim-sulfamethoxazole, glycopeptides (e.g., vancomycin, teicoplanin, and others), chloramphenicol, ansamycins (e.g., geldanamycin and others), streptogramins (e.g., pristinamycin IIA, pristinamycin IA, and others), sulfonamides (e.g., prontosil, sulfanilamide, sulfadiazine, sulfisoxazole, and others), tetracyclines (e.g., tetracycline, doxycycline, limecycline, oxytetracycline, and others), macrolides (e.g., erythromycin, clarithromycin, azithromycin, and others), oxazolidinones (e.g., linezolid, posizolid, tedizolid, cycloserine, and others), quinolones (e.g., ciprofloxacin, levofloxacin, trovafloxacin, and others), and lipopeptides (e.g., daptomycin, surfactin, and others).

In some embodiments, the compounds and compositions described herein are administered to a mammalian subject (e.g., human patient) in need of treatment for a fungal infection as adjuvant therapy of an antifungal agent, including but not limited to azoles (e.g. imidazole, ketoconazole, clomitrazole, fluconazole, itraconazole, posaconazole, voriconazole, isavuconazole, and others), polyenes (e.g. amphotericin B, nystatin, and others), echinocandins (e.g. nidulafungin, caspofungin, micafungin, and others) and flucytosine.

The present invention contemplates the use of compounds described herein for the treatment of bacterial infections by, for example, gram negative bacteria, Mycobateria (Mycobacterium tuberculosis, Mycobacterium leprae, Mycobacterium avium complex, etc.), and Neisseria meningitides. The present invention also contemplates the use of compounds described herein for “difficult-to-treat” gram negative bacterial infections, in which bacterial strains have acquired multidrug resistance (e.g., MRSA). Non-limiting examples of the gram negative bacterial infections to which the present invention can be applied include infections by Serratia marcescens; Salmonella typhimurium; Salmonella choleraesuis; Acinetobacter baumannii; Citrobacter freundii; Pseudomonas aeruginosa; Escherichia coli; Stenotrophomonas maltophilia; Enterobacter cloacae; Enterobacter aerogenes; Staphylococcus aureus and Klebsiella pneumoniae. When combined antibacterial drugs such as novobiocin and rifampicin, for example, the compounds of the present inventions demonstrate increased growth inhibition or cytotoxicity against gram negative bacterial cells as compared to when the antibiotics were used alone. These properties of the compounds are highly desirable as antibacterial therapy against bacterial infections, including multidrug-resistant gram negative bacterial infections (e.g., MRSA).

Another challenging area in medicine is the treatment of gram positive bacteria. Staphylococcus aureus (staph) is a gram-positive bacteria that about 30% of people carry in their nasal cavity. Most of the time, staph infections are benign; however, sometimes staph causes infections that can have serious health concerns. In healthcare settings, these staph infections can be serious or fatal, including bacteremia or sepsis when bacteria spread to the bloodstream, pneumonia, which most often affects people with underlying lung disease including those on mechanical ventilators, endocarditis (infection of the heart valves), which can lead to heart failure or stroke, or osteomyelitis (bone infection), which can be caused by staph bacteria traveling in the bloodstream or put there by direct contact such as following trauma (puncture wound of foot or intravenous (IV) drug abuse). Staph infections can be particularly serious health concern when caused by antibiotic resistant strains such as methicillin-resistant Staphylococcus aureus (MRSA) or vancomycin-resistant Staphylococcus aureus (VRSA).

The present invention also contemplates the use of compounds described herein for the treatment of bacterial infections by, for example, gram positive bacteria. Non-limiting examples of the gram positive bacterial infections to which the present invention can be applied include infections by Staphylococcus aureus.

Fungal infections can also be a serious health concern. Antifungal resistance is an increasing problem with the fungus Candida, a yeast. Candida infections may resist antifungal drugs, making them difficult to treat and are a health care concern. About 7% of all Candida blood samples tested at CDC are resistant to the antifungal drug fluconazole. Although one Candida species, Candida albicans, is the most common cause of severe Candida infections, resistance is most common in other species, particularly Candida auris, Candida glabrata, and Candida parapsilosis (Toda et al. MMWR Surveill Summ 2019; 68:1-15.). Resistance to another class of antifungal drugs, echinocandins, is particularly concerning. Echinocandin resistance appears to be increasing, especially in the species Candida glabrata. C. glabrata already has high levels of resistance to the antifungal fluconazole, and this resistance has remained fairly constant over the past 20 years, according to CDC surveillance data (Toda et al. MMWR Surveill Summ 2019; 68:1-15.). Echinocandins are the preferred treatment for C. glabrata, and echinocandin resistance could severely limit treatment options for patients with candidiasis caused by C. glabrata. Patients with Candida infections that are resistant to both fluconazole and echinocandin drugs have very few treatment options. The primary treatment option is amphotericin B, a drug that can be toxic for patients who are already very sick. Growing evidence suggests that patients who have drug-resistant Candida bloodstream infections (also known as candidemia) are less likely to survive than patients who have candidemia that can be treated by antifungal drugs (Alexander et al. Clin Infect Dis 2013; 56:1724-32 June 15; Baddley et al. Antimicrob Agents Chemother 2008;52:3022-8). In some embodiments, the mammalian subject of the present invention is a patient with Candida infections that are resistant to other drugs, for example, fluconazole and echinocandin drugs.

Concern is rising over the emerging fungus Candida auris (Satoh et al. Microbiol Immunol 2009;53:41-4.), which is rare in most areas of the United States but is a growing threat. Resistance rates for C. auris are much higher than for other Candida species, with about 90% of U.S. C. auris samples being resistant to fluconazole, up to one-third are resistant to the antifungal drug amphotericin B (Lockhart et al. Clin Infect Dis 2017;64:134-40.), and although most C. auris samples are susceptible to echinocandins, resistance to echinocandin drugs can also develop while the patient is being treated with these types of drugs. Moreover, C. auris is a concerning public health issue especially because it can be difficult to identify with standard laboratory methods and spreads easily in healthcare settings, such as hospitals and long-term care facilities with patients who have high care needs.

The present invention further contemplates the use of compounds described herein for the treatment of fungal infections. Non-limiting examples of fungal infections to which the present invention can be applied include infections by Candida parapsilosis, Candida krusei, Paecilomyces variotii, Candida albicans, Aspergillus fumigatus, Blastomyces dermatitidis, Candida auris, Candida glabrata, Candida guilliermondii, and Cryptococcus neoformans.

The compounds of the present disclosure or their pharmaceutically acceptable salts are generally administered in a therapeutically effective amount. The term “therapeutically effective amount” may refer to the amount (or dose) of a compound or other therapy that is necessary and sufficient to prevent, reduce, ameliorate, treat or eliminate a condition, or risk thereof, when administered to a subject in need of such compound or other therapy. The amount of the compound actually administered to a subject may be determined by a physician or caregiver, in the light of the relevant circumstances, including the condition to be treated, the chosen route of administration, the compound administered and its relative activity, the age, weight, the response of the individual patient, the severity of the patient's symptoms, and the like. Thus, the therapeutically effective amount may vary, for example, it may vary depending upon the subject's condition, the weight and age of the subject, the severity of the disease condition, the manner of administration and the like.

The compounds of the current disclosure may be administered by any of the accepted modes of administration, for example, by oral, cutaneous, topical, intradermal, intrathecal, intravenous, subcutaneous, intramuscular, intra-articular, intraspinal, spinal, nasal, epidural, inhalation by aerosol, rectal, vaginal or transdermal/transmucosal routes. A suitable route will depend on the nature and severity of the condition being treated. Oral administration may be a primary route of administration for compounds of the present disclosure as they generally exhibit increased oral bioavailability as well as enhanced organ targeting in combination of reduced in vivo toxicity. In some embodiments, topical administration is a route of administration for the compounds of this disclosure. In some embodiments, fungal infections are in the skin and can be addressed by topical applications of the compounds of this disclosure.

Intravenous (IV) administration can be a route of administration for the compounds of this disclosure. Intramuscular (IM) administration can be a route of administration for the compounds of this disclosure. Subcutaneous (SC) administration can be a route of administration for the compounds of this disclosure. Sublingual, or percutaneous administration can be also contemplated as a route of administration for the compounds of the present disclosure. Sublingual administration may be implemented with an appropriate formulation for the compounds. Inhalation of the compound of the current disclosure can be employed for a route of administration with an appropriate formulation (e.g., aerosol) for the treatment of bacterial or fungal infection in lungs (e.g., pulmonary infection). In some embodiments, a compound, or a stereoisomer or pharmaceutically acceptable salt, or a composition as described herein is administered to a human patient orally. In some embodiments, a compound, or a stereoisomer or pharmaceutically acceptable salt, or a composition as described herein is administered to a human patient intravenously. In some embodiments, a compound, or a stereoisomer or pharmaceutically acceptable salt, or a composition as described herein is administered to a human patient intramuscularly. In some embodiments, a compound, or a stereoisomer or pharmaceutically acceptable salt, or a composition as described herein is administered to a human patient subcutaneously. In some embodiments, a compound, or a stereoisomer or pharmaceutically acceptable salt, or a composition as described herein is administered to a human patient by inhalation as an aerosol.

In a particular example, the pharmaceutical composition provided herein may be administered to a human patient orally at a dose about 0.1 mg per kg to about 300 mg per kg or to even 500 mg per kg. In one embodiment, the pharmaceutical composition provided herein may be administered to a human patient orally at a dose about 1 mg per kg to about 300 mg per kg daily. In another example, the pharmaceutical composition provided herein may be administered to a human patient orally at a dose about 1 mg per kg to about 100m per kg.

Compounds of the present disclosure, or a stereoisomer or pharmaceutically acceptable salt thereof can be administered to a subject (e.g., human patient) suffering from a bacterial or fungal infection, e.g., orally, intravenously, intramuscularly or subcutaneously at a dose of, for example, about 0.5 mg per kg, 0.6 mg per kg, about 0.7 mg per kg, about 0.8 mg per kg, about 0.9 mg per kg, about 1 mg per kg, about 2 mg per kg, about 3 mg per kg, about 4 mg per kg, about 5 mg per kg, about 6 mg per kg, about 7 mg per kg, about 8 mg per kg, about 9 mg per kg, about 10 mg per kg, about 15 mg per kg, about 20 mg per kg about 30 mg per kg, about 40 mg per kg, about 50 mg per kg, about 60 mg per kg, about 70 mg per kg, about 80 mg per kg, about 90 mg per kg, about 100 mg per kg, about 110 mg per kg, about 120 mg per kg, about 130 mg per kg, about 140 mg per kg, about 150 mg per kg, about 160 mg per kg, about 170 mg per kg, about 180 mg per kg, about 190 mg per kg, about 200 mg per kg, about 210 mg per kg, about 220 mg per kg, about 230 mg per kg, about 240 mg per kg, about 250 mg per kg, about 260 mg per kg, about 270 mg per kg, about 280 mg per kg, about 290 mg per kg, about 300 mg per kg, about 350 mg per kg, about 400 mg per kg, about 450 mg per kg, about 500 mg per kg, or about 600 mg per kg.

In one embodiment, compounds of the present disclosure or a stereoisomer or pharmaceutically acceptable salt thereof can be administered orally at a dose of, for example, about 0.5 mg per kg, 0.6 mg per kg, about 0.7 mg per kg, about 0.8 mg per kg, about 0.9 mg per kg, about 1 mg per kg, about 2 mg per kg, about 3 mg per kg, about 4 mg per kg, about 5 mg per kg, about 6 mg per kg, about 7 mg per kg, about 8 mg per kg, about 9 mg per kg, about 10 mg per kg, about 15 mg per kg, about 20 mg per kg, about 30 mg per kg, about 40 mg per kg, about 50 mg per kg, about 60 mg per kg, about 70 mg per kg, about 80 mg per kg, about 90 mg per kg, about 100 mg per kg, about 110 mg per kg, about 120 mg per kg, about 130 mg per kg, about 140 mg per kg, about 150 mg per kg, about 160 mg per kg, about 170 mg per kg, about 180 mg per kg, about 190 mg per kg, about 200 mg per kg, about 210 mg per kg, about 220 mg per kg, about 230 mg per kg, about 240 mg per kg, about 250 mg per kg, about 260 mg per kg, about 270 mg per kg, about 280 mg per kg, about 290 mg per kg, about 300 mg per kg, about 350 mg per kg, about 400 mg per kg, about 450 mg per kg, about 500 mg per kg, or about 600 mg per kg.

In one embodiment, compounds of the present disclosure or a stereoisomer or pharmaceutically acceptable salt thereof can be administered intravenously at a dose of, for example, 0.5 mg per kg, 0.6 mg per kg, about 0.7 mg per kg, about 0.8 mg per kg, about 0.9 mg per kg, about 1 mg per kg, about 2 mg per kg, about 3 mg per kg, about 4 mg per kg, about 5 mg per kg, about 6 mg per kg, about 7 mg per kg, about 8 mg per kg, about 9 mg per kg, about 10 mg per kg, about 15 mg per kg, about 20 mg per kg, about 25 mg per kg, about mg per kg, about 35 mg per kg, about 40 mg per kg, about 50 mg per kg, about 60 mg per kg, about 70 mg per kg, about 80 mg per kg, about 90 mg per kg, about 100 mg per kg, about 110 mg per kg, about 120 mg per kg, about 130 mg per kg, about 140 mg per kg, about 150 mg per kg, about 160 mg per kg, about 170 mg per kg, about 180 mg per kg, about 190 mg per kg, about 200 mg per kg, about 210 mg per kg, about 220 mg per kg, about 230 mg per kg, about 240 mg per kg, about 250 mg per kg, about 260 mg per kg, about 270 mg per kg, about 280 mg per kg, about 290 mg per kg, or about 300 mg per kg.

In one embodiment, compounds of the present disclosure or a stereoisomer or pharmaceutically acceptable salt thereof can be administered subcutaneously at a dose of, for example, 0.5 mg per kg, 0.6 mg per kg, about 0.7 mg per kg, about 0.8 mg per kg, about 0.9 mg per kg, about 1 mg per kg, about 2 mg per kg, about 3 mg per kg, about 4 mg per kg, about 5 mg per kg, 6 mg per kg, about 7 mg per kg, about 8 mg per kg, about 9 mg per kg, about 10 mg per kg, about 15 mg per kg, about 20 mg per kg, about 30 mg per kg, about 40 mg per kg, about 50 mg per kg, about 60 mg per kg, about 70 mg per kg, about 80 mg per kg, about 90 mg per kg, about 100 mg per kg, about 110 mg per kg, about 120 mg per kg, about 130 mg per kg, about 140 mg per kg, about 150 mg per kg, about 160 mg per kg, about 170 mg per kg, about 180 mg per kg, about 190 mg per kg, about 200 mg per kg, about 210 mg per kg, about 220 mg per kg, about 230 mg per kg, about 240 mg per kg, about 250 mg per kg, about 260 mg per kg, about 270 mg per kg, or about 280 mg per kg, about 290 mg per kg, or about 300 mg per kg.

In one embodiment, compounds of the present disclosure or a stereoisomer or pharmaceutically acceptable salt thereof can be administered orally to a subject suffering from a bacterial or fungal infection at a dose of, for example, about 0.5 mg per kg, 0.6 mg per kg, about 0.7 mg per kg, about 0.8 mg per kg, about 0.9 mg per kg, about 1 mg per kg, about 2 mg per kg, about 3 mg per kg, about 4 mg per kg, about 5 mg per kg, about 6 mg per kg, about 7 mg per kg, about 8 mg per kg, about 9 mg per kg, about 10 mg per kg, about 15 mg per kg, about 20 mg per kg, about 30 mg per kg, about 40 mg per kg, about 50 mg per kg, about 60 mg per kg, about 70 mg per kg, about 80 mg per kg, about 90 mg per kg, about 100 mg per kg, about 110 mg per kg, about 120 mg per kg, about 130 mg per kg, about 140 mg per kg, about 150 mg per kg, about 160 mg per kg, about 170 mg per kg, about 180 mg per kg, about 190 mg per kg, about 200 mg per kg.

The administration can be three times a day, twice a day, once a day, once in two days, once in three days, once in four days, once in five days, once in six days, once in a week, once in ten days, or once in two weeks. The administration can also include dosing holidays from about 1 day to about 7 days between administration.

In some embodiments, compounds of the present disclosure or a stereoisomer or pharmaceutically acceptable salt thereof are administered at a dose of 1 mg per kg to about 200 mg per kg daily. In some embodiments, compounds of the present disclosure or a stereoisomer or pharmaceutically acceptable salt thereof are administered at a dose of 1 mg per kg to about 100 mg per kg daily. In some embodiments, compounds of the present disclosure or a stereoisomer or pharmaceutically acceptable salt thereof are administered at a dose of 1 mg per kg to about 50 mg per kg daily. In some embodiments, compounds of the present disclosure or a stereoisomer or pharmaceutically acceptable salt thereof are administered at a dose of 0.5 mg per kg to about 50 mg per kg daily. In some embodiments, compounds of the present disclosure or a stereoisomer or pharmaceutically acceptable salt thereof are administered at a dose of 10 mg per kg to about 20 mg per kg daily.

The subject according to the present invention is typically a mammal (e.g., a human patient) diagnosed as being in need of treatment for a bacterial infection by, for example, Serratia marcescens, Salmonella typhimurium, Salmonella choleraesuis, Acinetobacter baumannii, Citrobacter freundii, Pseudomonas aeruginosa, Escherichia coli, Stenotrophomonas maltophilia, Enterobacter cloacae, Enterobacter aerogenes, Mycobacterium tuberculosis, Mycobaterium avium complex, Mycobacterium leprae, Neisseria meningitidis, Staphylococcus aureus and Klebsiella pneumoniae, or a fungal infection, by, for example Candida parapsilosis, Candida krusei, Paecilomyces variotii, Candida albicans, Aspergillus fumigatus, Blastomyces dermatitidis, Candida auris, Candida glabrata, Candida guilliermondii, and Cryptococcus neoformans. The present invention contemplates that the subject is being treated with novobiocin, rifampicin or other antibiotics, for example, cephalosporins (e.g., ceftriaxone-cefotaxime, ceftazidime, and others), fluoroquinolones (e.g., ciprofloxacin, levofloxacin and others), aminoglycosides (e.g., gentamicin, amikacin and others), imipenem, broad-spectrum penicillins with or without β-lactamase inhibitors (e.g., amoxicillin-clavulanic acid, piperacillin-tazobactam and others), trimethoprim-sulfamethoxazole, glycopeptides (e.g., vancomycin, teicoplanin, and others), chloramphenicol, ansamycins (e.g., geldanamycin and others), streptogramins (e.g., pristinamycin IIA, pristinamycin IA, and others), sulfonamides (e.g., prontosil, sulfanilamide, sulfadiazine, sulfisoxazole, and others), tetracyclines (e.g., tetracycline, doxycycline, limecycline, oxytetracycline, and others), macrolides (e.g., erythromycin, clarithromycin, azithromycin, and others), oxazolidinones (e.g., linezolid, posizolid, tedizolid, cycloserine, and others), quinolones (e.g., ciprofloxacin, levofloxacin, trovafloxacin, and others), and lipopeptides (e.g., daptomycin, surfactin, and others). The present invention also contemplates that the subject is being treated with other antifungal compounds, for example azoles (e.g. imidazole, ketoconazole, clomitrazole, fluconazole, itraconazole, posaconazole, voriconazole, isavuconazole, and others), polyenes (e.g. amphotericin B, nystatin, and others), echinocandins (e.g. nidulafungin, caspofungin, micafungin, and others) and flucytosine.In addition to the methods for treating bacterial or fungal infections, the compounds and compositions described herein can be used to treat a subject in need of treatment for a cell proliferation disorder such as cancer. Non-limiting examples of cancer include liver cancer, cholangiocarcinoma, osteosarcoma, melanoma, breast cancer, renal cancer, prostate cancer, gastric cancer, colorectal cancer, thyroid cancer, head and neck cancer, ovarian cancer, pancreatic cancer, neuronal cancer, lung cancer, uterine cancer, leukemia, or lymphoma. The subject is typically a mammal diagnosed as being in need of treatment for one or more of such proliferative disorders, particularly a human patient. The methods comprise administering an effective amount of at least one compound of the invention; optionally the compound may be administered in combination with one or more additional therapeutic agents, particularly the therapeutic agents known to be useful for treating the cancer or proliferative disorder afflicting the particular subject.

### Combination Therapy

The disclosure provided herein describes methods to treat bacterial infections, fungal infections, or cancer in a subject by administering to a subject at least one compound of the present disclosure. The methods disclosed herein can further comprise administering to the subject a combination of a compound of Formulae (I′) (I), (II), (I′-a), (I′-b), or (I′-c), or a stereoisomer or pharmaceutically acceptable salt thereof and at least one additional antibiotic, antifungal, or anticancer agent wherein the combined composition may be administered as a co-formulation or separately. In some embodiments, a compound, or a stereoisomer or pharmaceutically acceptable salt, or a composition as described herein is administered to a human patient simultaneously or subsequently.

In certain particular embodiments, more than one compound of the current disclosure may be administered at a time to the subject. In some embodiments, two compounds of the current disclosure may act synergistically or additively, and either compound may be used in a lesser amount than if administered alone.

In some embodiments, compounds disclosed herein and/or pharmaceutical compositions thereof are administered concurrently with the administration of another therapeutic agent. For example, compounds disclosed herein and/or pharmaceutical rapositimis thereof may be administered together with another therapeutic agent. In other embodiments, compounds disclosed herein and/or pharmaceutical compositions thereof are administered prior or subsequent to administration of other therapeutic agents.

In some embodiments, compounds disclosed herein and/or pharmaceutical compositions thereof are synergistic with an active therapeutic agent in growth inhibition of bacterial strains. The active therapeutic agent(s) used in combination therapy can be an antibacterial agent such as rifampicin, novobiocin, clorobiocin, coumermycin A1, cephalosprorins, and penicillins. The antibacterial agent used in combination therapy can be any antibacterial agent used in treatment of methicillin-resistant Staphylococcus aureus (“MRSA”) infection or other difficult-to-treat infections in humans. The present invention contemplates the use in conjunction with any antibacterial agent, for example, cephalosporins (e.g., ceftriaxone-cefotaxime, ceftazidime, and others), fluoroquinolones (e.g., ciprofloxacin, levofloxacin and others), aminoglycosides (e.g., gentamicin, amikacin and others), imipenem, broad-spectrum penicillins with or without β-lactamase inhibitors (e.g., amoxicillin-clavulanic acid, piperacillin-tazobactam and others), trimethoprim-sulfamethoxazole, glycopeptides (e.g., vancomycin, teicoplanin, and others), chloramphenicol, ansamycins (e.g., geldanamycin and others), streptogramins (e.g., pristinamycin IIA, pristinamycin IA, and others), sulfonamides (e.g., prontosil, sulfanilamide, sulfadiazine, sulfisoxazole, and others), tetracyclines (e.g., tetracycline, doxycycline, limecycline, oxytetracycline, and others), macrolides (e.g., erythromycin, clarithromycin, azithromycin, and others), oxazolidinones (e.g., linezolid, posizolid, tedizolid, cycloserine, and others), quinolones (e.g., ciprofloxacin, levofloxacin, trovafloxacin, and others), and lipopeptides (e.g., daptomycin, surfactin, and others).

In some embodiments, compounds disclosed herein and/or pharmaceutical rapositions thereof are used in combination with an additional active therapeutic agent in growth inhibition of fungal strains. The active therapeutic agent(s) used in combination therapy can be an antifungal agent such as azoles (e.g. imidazole, ketoconazole, clomitrazole, fluconazole, itraconazole, posaconazole, voriconazole, isavuconazole, and others), polyenes (e.g. amphotericin B, nystatin, and others), echinocandins (e.g. nidulafungin, caspofungin, micafungin, and others) and flucytosine. Without being bound by any particular theory, the active therapeutic agents may target the ergosterol biosynthesis pathway which inhibits membrane integrity, inhibit beta(1-3) synthase which disrupts the cell wall, and/or inhibits DNA and RNA synthesis. In some embodiments, antifungal agents which disrupt membrane synthesis (including, but not limited to azoles, polyenes, and echinocandins) are used in combination with the compounds disclosed herein and/or pharmaceutical compositions thereof.

### Pharmaceutical Formulations

The compounds of the current disclosure may be administered by any of the accepted modes of administration of agents having similar utilities, for example, by oral, cutaneous, topical, intradermal, intrathecal, intravenous, subcutaneous, intramuscular, intra-articular, intraspinal or spinal, nasal, epidural, or transdermal/transmucosal inhalable routes via aerosol formulation. In one particular example, the compounds of the current disclosure may be formulated in aerosol for the treatment of pulmonary infections.

In one particular example, the pharmaceutical composition can be administered to a patient orally. In another particular example, the pharmaceutical composition comprising a pentamidine analog disclosed herein or a stereoisomer or pharmaceutically acceptable salt thereof may be administered to a patient intravenously (e.g., injection or infusion). In another particular example, the pharmaceutical composition may be administered to a patient intramuscularly. In a particular example, the pharmaceutical composition may be administered to a patient nasally. A pharmaceutical composition (e.g., for oral administration, inhalation, injection, infusion, subcutaneous delivery, intramuscular delivery, intraperitoneal delivery, sublingual delivery, or other methods) may be in the form of a liquid. A liquid pharmaceutical composition may include, for example, one or more of the following: a sterile diluent such as water, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral composition can be enclosed in ampoules, disposable syringes or multiple dose vials made of glass or plastic. The use of physiological saline is preferred, and an injectable pharmaceutical composition is preferably sterile. A liquid pharmaceutical composition may be delivered orally.

A pharmaceutical composition comprising a compound of Formulae (I′) (I), (II), (I′-a), (I′-b), or (I′-c), or a stereoisomer or pharmaceutically acceptable salt thereof may be formulated for sustained or slow release (also called timed release or controlled release). Such compositions can be prepared using well known technology and administered by, for example, oral, rectal, intradermal, or subcutaneous implantation, or by implantation at the desired target site. Sustained-release formulations may contain the compound dispersed in a carrier matrix and/or contained within a reservoir surrounded by a rate controlling membrane. Excipients for use within such formulations are biocompatible, and may be biodegradable; preferably, the formulation provides a relatively constant level of active component release. Non-limiting examples of excipients include water, alcohol, glycerol, chitosan, alginate, chondroitin, Vitamin E, mineral oil, and dimethyl sulfoxide (DMSO). The amount of compound contained within a sustained release formulation depends upon the site of implantation, the rate and expected duration of release, and the nature of the condition, disease or disorder to be treated or prevented.

The pharmaceutical composition comprising one or more pentamidine analogs or a stereoisomer or pharmaceutically acceptable salt thereof may be effective over time. In some cases, the pharmaceutical composition may be effective for one or more days. In some cases, the duration of efficacy of the pharmaceutical composition is over a long period of time. In some cases, the efficacy of the pharmaceutical composition may be greater than 2 days, 3 days, 4 days, 5 days, 6 days, 1 week, 2 weeks, 3 weeks, or 1 month.

In making the pharmaceutical composition comprising one or more pentamidine analogs or a stereoisomer or pharmaceutically acceptable salt thereof, the active ingredient can be diluted by an excipient. Some examples of suitable excipients include lactose, dextrose, sucrose, sorbitol, mannitol, starches, gum acacia, calcium phosphate, alginates, tragacanth, gelatin, calcium silicate, microcrystalline cellulose, PEG, polyvinylpyrrolidone, cellulose, water, sterile saline, syrup, and methyl cellulose. The compositions of the disclosure can be formulated so as to provide quick, sustained or delayed release of the active ingredient after administration to the patient by employing procedures known in the art. In some cases, the pharmaceutical composition comprising a pentamidine analog or a stereoisomer or pharmaceutically acceptable salt thereof may comprise an excipient that can provide long term preservation, bulk up a formulation that contains a potent active ingredient, facilitate drug absorption, reduce viscosity, add flavoring, or enhance the solubility of the pharmaceutical composition.

In some cases, the pharmaceutical composition comprising a pentamidine analog or a stereoisomer or pharmaceutically acceptable salt thereof may comprise a pharmaceutically acceptable carrier. The pharmaceutically acceptable carrier may include any and all solvents, dispersion media, coatings, antibacterial and antifungal agents, isotonic and absorption delaying agents, and the like that are physiologically compatible. Preferably, the carrier is suitable for oral administration. The active compound may be coated in a material to protect the compound from the action of acids and other natural conditions that may inactivate the compound. The carrier can be suitable for parenteral (e.g., intravenous, intramuscular, subcutaneous, intrathecal) administration (e.g., by injection or infusion).

The present invention also contemplates formulating a pharmaceutically acceptable salt of a compound of Formulae (I′) (I), (II), (I′-a), (I′-b), or (I′-c). In general, pharmaceutical salts may include, but are not included, salts and base addition salts (e.g., hydrochloric acid salt, dihydrocholoric acid salt, sulfuric acid salt, citrate, hydrobromic acid salt, hydroiodic acid salt, nitric acid salt, bisulfate, phosphoric acid salt, super phosphoric acid salt, isonicotinic acid salt, acetic acid salt, lactic acid salt, salicylic acid salt, tartaric acid salt, pantothenic acid salt, ascorbic acid salt, succinic acid salt, maleic acid salt, fumaric acid salt, gluconic acid salt, saccharinic acid salt, formic acid salt, benzoic acid salt, glutaminic acid salt, methanesulfonic acid salt, ethanesulfonic acid salt, benzenesulfonic acid salt, p-toluenesulfonic acid salt, pamoic acid salt (pamoate)), as well as salts of aluminum, calcium, lithium, magnesium, calcium, sodium, zinc, and diethanolamine.

### Kits

The invention further provides kits for carrying out the methods of the invention, which comprises one or more compounds described herein, or a stereoisomer or pharmaceutically acceptable salt thereof, or a pharmaceutical composition comprising a compound described herein, or a stereoisomer or pharmaceutically acceptable salt thereof. The kits may employ any of the compounds disclosed herein, or a stereoisomer or pharmaceutically acceptable salt thereof. The kits may be used for any one or more of the uses described herein, and, accordingly, may contain instructions for use in the treatment of bacterial or fungal infections, or cancer.

Kits generally comprise suitable packaging. The kits may comprise one or more containers comprising any compound described herein. Each component (if there is more than one component) can be packaged in separate containers or some components can be combined in one container where cross-reactivity and shelf life permit. One or more components of a kit may be sterile and/or may be contained within sterile packaging.

The kits may be in unit dosage forms, bulk packages (e.g., multi-dose packages) or sub-unit doses. For example, kits may be provided that contain sufficient dosages of a compound as disclosed herein (e.g., a therapeutically effective amount) and/or a second pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to an individual.

The following enumerated embodiments are representative of some aspects of the invention.

Embodiment 1. A compound comprising Formula (I)

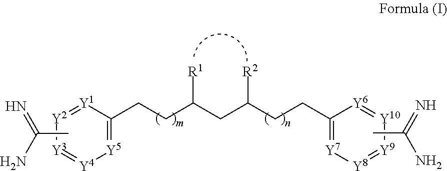

wherein:

m or n is independently an integer of 0, 1, 2 or 3;

R¹ and R² are independently hydrogen or halo, or R¹ taken together with R² forms a saturated, unsaturated or partially unsaturated 3-9 member ring

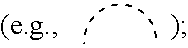

and

Y¹ through Y¹⁰ are independently CR³, wherein R³ is independently hydrogen, heterocycle, or amidine

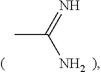

wherein R³ is hydrogen at positions Y¹, Y⁵, Y⁶ and Y⁷, amidine

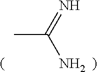

at one of Y², Y³ or Y⁴ and optionally at one of Y⁸, Y⁹, or Y¹⁰;

or a pharmaceutically acceptable salt thereof.

Embodiment 2. The compound of Embodiment 1, wherein m is 1, and n is 1.

Embodiment 3. The compound of Embodiment 1, wherein in is 1, and n is 0.

Embodiment 4. The compound of Embodiment 1, wherein rn is 0, and n is 1.

Embodiment 5. The compound of Embodiment 1, wherein in is 1, and n is 2.

Embodiment 6. The compound of Embodiment 1, wherein rn is 2, and n is 1.

Embodiment 7. The compound of Embodiment 1, wherein in is 0, and n is 0.

Embodiment 8. The compound of Embodiment 1, wherein R¹ and R² are independently hydrogen.

Embodiment 9. The compound of Embodiment 1, wherein R¹ taken together with R² forms a saturated, unsaturated or partially unsaturated 3-9 member cyclic group (e.g.,)

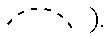

Embodiment 10. The compound of Embodiment 9, wherein R¹ taken together with R² forms 5 member saturated cycloalkyl.

Embodiment 11. The compound of Embodiment 9, wherein R¹ taken together with R² forms 6 member saturated cycloalkyl.

Embodiment 12. The compound of Embodiment 9, wherein R¹ taken together with R² forms 7 member saturated cycloalkyl.

Embodiment 13. The compound of Embodiment 1, wherein R¹ and R² are independently hydrogen, and R³ is amidine at Y⁴ and Y⁸.

Embodiment 14. The compound of Embodiment 1, wherein R¹ taken together with R² forms a saturated, unsaturated or partially unsaturated 3-9 member cyclic group; and R³ is amidine at Y⁴ and Y⁸ and hydrogen at Y², Y³, Y⁹, and Y¹⁰.

Embodiment 15. The compound of Embodiment 14, wherein R¹ taken together with R² forms a saturated 5 member cycloalkyl.

Embodiment 16. The compound of Embodiment 14, wherein R¹ taken together with R² forms a saturated 6 member cycloalkyl.

Embodiment 17. The compound of Embodiment 14, wherein R¹ taken together with R² forms a saturated 7 member cycloalkyl.

Embodiment 18. The compound of Embodiment 1, wherein R¹ and R² are independently hydrogen; and R³ is amidine at Y² and Y¹⁰ and hydrogen at Y³, Y⁴, Y⁸, and Y⁹.

Embodiment 19. The compound of Embodiment 1, wherein R¹ taken together with R² forms a saturated, unsaturated or partially unsaturated 3-9 member cyclic group

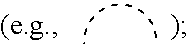

and R³ is amidine at Y² and Y¹⁰ and hydrogen at Y³, Y⁴, Y⁸, and Y⁹.

Embodiment 20. The compound of Embodiment 19, wherein R¹ taken together with R² forms a saturated 5 member cycloalkyl.

Embodiment 21. The compound of Embodiment 19, wherein R¹ taken together with R² forms a saturated 6 member cycloalkyl.

Embodiment 22. The compound of Embodiment 19, wherein R¹ taken together with R² forms a saturated 7 member cycloalkyl.

Embodiment 23. The compound of Embodiment 1, wherein R¹ and R² are independently hydrogen, and R³ is amidine at Y³ and Y⁹, and hydrogen at Y², Y⁴, Y⁸, and Y¹⁰.

Embodiment 24. The compound of Embodiment 1, wherein R¹ taken together with R² forms a saturated, unsaturated or partially unsaturated 3-9 member cyclic group

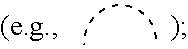

and R³ is amidine at Y³ and Y⁹, and hydrogen at Y², Y⁴, Y⁸, and Y¹⁰.

Embodiment 25. The compound of Embodiment 24, wherein R¹ taken together with R² forms 5 member cycloalkyl.

Embodiment 26. The compound of Embodiment 24, wherein R¹ taken together with R² forms 6 member cycloalkyl.

Embodiment 27. The compound of Embodiment 24, wherein R¹ taken together with R² forms 7 member cycloalkyl.

Embodiment 28. The compound of Embodiment 1, wherein R³ is a heterocycle at Y⁸, Y⁹, or Y¹⁰ when amidine

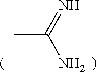

is not present at any one of Y⁸, Y⁹, or Y¹⁰.

Embodiment 29. The compound of Embodiment 1, wherein R¹ and R² are independently hydrogen and R³ is amidine

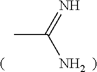

at Y², a saturated 5 member heterocycloalkyl at Y¹⁰, and hydrogen at Y³, Y⁴, Y⁸, and Y⁹.

Embodiment 30. The compound Embodiment 29, wherein the saturated 5 member heterocycloalkyl is pyrrolidinyl.

Embodiment 31. The compound of Embodiment 1, wherein R¹ and R² are independently hydrogen and R³ is amidine

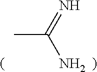

at Y³, a saturated 5 member heterocycloalkyl at Y⁹, and hydrogen at Y², Y⁴, Y⁸, and Y¹⁰.

Embodiment 32. The compound Embodiment 31, wherein the saturated 5 member heterocycloalkyl is pyrrolidinyl.

Embodiment 33. The compound of Embodiment 1, wherein R¹ and R² are independently hydrogen and R³ is amidine

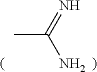

at Y⁴, a saturated 5 member heterocycloalkyl at Y⁸, and hydrogen at Y², Y³, Y⁹, and Y¹⁰.

Embodiment 34. The compound Embodiment 33, wherein the saturated 5 member heterocycloalkyl is pyrrolidinyl.

Embodiment 35. The compound of Embodiment 1, wherein R¹ taken together with R² forms a saturated, unsaturated or partially unsaturated 3-9 member cyclic group; and R³ is amidine

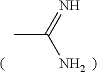

at Y³, a saturated 5 member heterocycloalkyl at Y⁹, and hydrogen at Y², Y⁴, Y⁸, and Y¹⁰.

Embodiment 36. The compound of Embodiment 35, wherein R¹ taken together with R² forms a saturated 5 member cycloalkyl.

Embodiment 37. The compound of Embodiment 35, wherein R¹ taken together with R² forms a saturated 6 member cycloalkyl.

Embodiment 38. The compound of Embodiment 35, wherein R¹ taken together with R² forms a saturated 7 member cycloalkyl.

Embodiment 39. The compound of Embodiment 35, wherein a saturated heterocycloalkyl at Y⁹ is pyrrolidinyl.

Embodiment 40. The compound of Embodiment 1, wherein R¹ taken together with R² forms a saturated 6 member cycloalkyl; and R³ is amidine

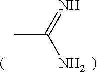

at Y³, pyrrolidinyl at Y⁹, and hydrogen at Y², Y⁴, Y⁸, and Y¹⁰.

Embodiment 41. The compound of Embodiment 1, wherein R¹ taken together with R² forms a saturated 6 member cycloalkyl; and R³ is amidine

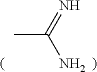

at Y², pyrrolidinyl at Y¹⁰, and hydrogen at Y³, Y⁴, Y⁸, and Y⁹.

Embodiment 42. The compound of Embodiment 1, wherein R¹ taken together with R² forms a saturated 6 member cycloalkyl; and R³ is amidine

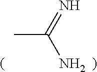

at Y⁴, pyrrolidinyl at Y⁸, and hydrogen at Y², Y³, Y⁹, and Y¹⁰.

Embodiment 43. The compound of Embodiment 1, wherein said compound of Formula (I) is selected from the group consisting of:

3,3′-(heptane-1,7-diyl)dibenzimidamide;

3-(7-(3-(pyrrolidin-3-yl)phenyl)heptyl)benzimidamide;

4,4′-(2,2′-((1R,3S)-cyclohexane-1,3-diyl)bis(ethane-2,1-diyl))dibenzimidamide;

4,4′-(2,2′-((1R,3R)-cyclohexane-1,3-diyl)bis(ethane-2,1-diyl))dibenzimidamide;

4-(2-((1S ,3R)-3-(4-(pyrrolidin-3-yl)phenethyl)cyclohexyl)ethyl)benzimidamide; and

3,3′ -(((1R,3S)-cyclohexane-1,3-diyl)bis(ethane-2,1-diyl))dibenzimidamide.

Embodiment 44. The compound of Embodiment 43, wherein said compound of Formula (I) is 4,4′-(2,2′-((1R,3S)-cyclohexane-1,3-diyl)bis(ethane-2,1-diyl))dibenzimidamide.

Embodiment 45. A compound according to the following formula:

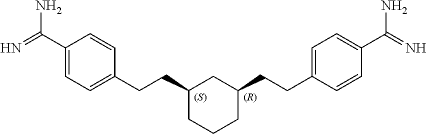

or a pharmaceutically acceptable salt thereof.

Embodiment 46. A compound according to the following formula:

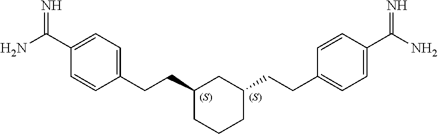

or a pharmaceutically acceptable salt thereof.

Embodiment 47. A compound according to the following formula:

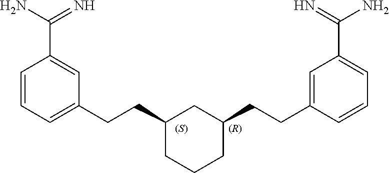

or a pharmaceutically acceptable salt thereof.

Embodiment 48. A compound according to the following formula:

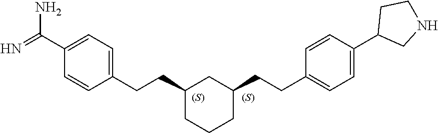

or a pharmaceutically acceptable salt thereof.

Embodiment 49. A compound according to the following formula:

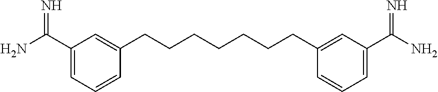 [SMILES: N=C(N)c1cccc(CCCCCCCc2cccc(C(=N)N)c2)c1]

or a pharmaceutically acceptable salt thereof.

Embodiment 50. A compound according to the following formula:

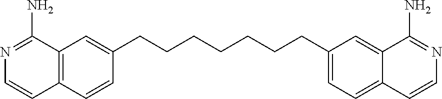 [SMILES: Nc1nccc2ccc(CCCCCCCc3ccc4ccnc(N)c4c3)cc12]

or a pharmaceutically acceptable salt thereof.

Embodiment 51. A compound according to the following formula:

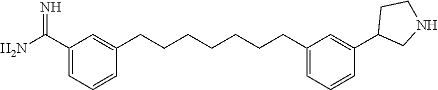 [SMILES: N=C(N)c1cccc(CCCCCCCc2cccc(C3CCNC3)c2)c1]

or a pharmaceutically acceptable salt thereof.

Embodiment 52. A compound according to the following formula:

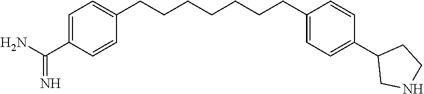 [SMILES: N=C(N)c1ccc(CCCCCCCc2ccc(C3CCNC3)cc2)cc1]

or a pharmaceutically acceptable salt thereof.

Embodiment 53. A method of treating a bacterial infection, the method comprising administering an effective amount of a compound as in any preceding Embodiments to a subject suffering from a bacterial infection.

Embodiment 54. The method of Embodiment 53, wherein said bacterial infection is a gram negative bacterial infection.

Embodiment 55. The method of Embodiment 53, wherein said bacterial infection is a bacterial infection caused by a strain selected from the group consisting of Serratia marcescens; Salmonella typhimurium, Salmonella choleraesuis, Acinetobacter baumannii, Citrobacter freundii, Pseudomonas aeruginosa; Stenotrophomonas maltophilia, Enterobacter cloacae, Enterobacter aerogene, Mycobacterium tuberculosis, Mycobacterium leprae, Mycobacterium avium complex, Staphylococcus aureus, keisseria Klebsiella pneumoniae, and Klebsiella pneumoniae.

Embodiment 56. The method of Embodiment 53, wherein said subject is a human patient.

Embodiment 57. The method of Embodiment 56, wherein said subject is suffering from a methicillin-resistant Staphylococcus aureus (MRSA) infection, tuberculosis, or meningitidis.

Embodiment 58. The method of Embodiment 56, wherein said compound is administered to the human patient via inhalation using aerosol.

Embodiment 59. The method of Embodiment 53, wherein said subject is suffering from a lung infection.

Embodiment 60. The method of Embodiment 53, wherein said compound is administered to the subject (e.g., human patient) orally, intravenously, intramuscularly, or subcutaneously at a dose of about 0.5 mg per kg, 0.6 mg per kg, about 0.7 mg per kg, about mg per kg, about 0.9 mg per kg, about 1 mg per kg, about 2 mg per kg, about 3 mg per kg, about 4 mg per kg, about 5 mg per kg, about 6 mg per kg, about 7 mg per kg, about 8 mg per kg, about 9 mg per kg, about 10 mg per kg, about 15 mg per kg, about 20 mg per kg about mg per kg, about 40 mg per kg, about 50 mg per kg, about 60 mg per kg, about 70 mg per kg, about 80 mg per kg, about 90 mg per kg, about 100 mg per kg, about 110 mg per kg, about 120 mg per kg, about 130 mg per kg, about 140 mg per kg, about 150 mg per kg, about 160 mg per kg, about 170 mg per kg, about 180 mg per kg, about 190 mg per kg, about 200 mg per kg, about 210 mg per kg, about 220 mg per kg, about 230 mg per kg, about 240 mg per kg, about 250 mg per kg, about 260 mg per kg, about 270 mg per kg, about 280 mg per kg, about 290 mg per kg, about 300 mg per kg, about 350 mg per kg, about 400 mg per kg, about 450 mg per kg, about 500 mg per kg, or about 600 mg per kg.

Embodiment 61. The method of Embodiment 60, wherein said compound is administered to a human patient orally.

Embodiment 62. The method of Embodiment 60, wherein said compound is administered to a human patient intravenously.

Embodiment 61. The method of Embodiment 60, wherein said compound is administered to a human patient intramuscularly.

Embodiment 62. The method of Embodiment 60, wherein said compound is administered to a human patient subcutaneously.

Embodiment 63. The method of Embodiment 53, wherein said subject is administered about 1 mg per kg to about 200 mg per kg daily.

Embodiment 64. The method of Embodiment 53, wherein said subject is administered about 1 mg per kg to about 100 mg per kg daily.

Embodiment 65. The method of Embodiment 53, wherein said subject is administered about 1 mg per kg to about 50 mg per kg daily.

Embodiment 66. The method of Embodiment 53, wherein said subject is administered about 0.5 mg per kg to about 50 mg per kg daily.

Embodiment 67. The method of Embodiment 53, wherein said subject is administered about 10 to about 20 mg per kg daily.

Embodiment 68. The method of Embodiment 53, wherein said subject is a human patient suffering from a gram negative bacterial infection and being treated with an antibiotic drug.

Embodiment 69. The method of Embodiment 70, wherein said antibiotic drug is novobiocin.

Embodiment 70. The method of Embodiment 70, wherein said antibiotic drug is rifampicin.

Embodiment 71. The method of Embodiment 70, wherein said human patient is administered with a compound according to Embodiment 1 at about 0.5 mg per kg to about 50 mg per kg daily.

Embodiment 72. The method of Embodiment 70, wherein said antibiotic drug is selected from the group consisting of novobiocin, rifampicin, cephalosporins (e.g., ceftriaxone-cefotaxime, ceftazidime, and others), fluoroquinolones (e.g., ciprofloxacin, levofloxacin and others), aminoglycosides (e.g., gentamicin, amikacin and others), imipenem, broad-spectrum penicillins with or without β-lactamase inhibitors (e.g., amoxicillin-clavulanic acid, piperacillin-tazobactam and others), trimethoprim-sulfamethoxazole, glycopeptides (e.g., vancomycin, teicoplanin, and others), chloramphenicol, ansamycins (e.g., geldanamycin and others), streptogramins (e.g., pristinamycin IIA, pristinamycin IA, and others), sulfonamides (e.g., prontosil, sulfanilamide, sulfadiazine, sulfisoxazole, and others), tetracyclines (e.g., tetracycline, doxycycline, limecycline, oxytetracycline, and others), macrolides (e.g., erythromycin, clarithromycin, azithromycin, and others), oxazolidinones (e.g., linezolid, posizolid, tedizolid, cycloserine, and others), quinolones (e.g., ciprofloxacin, levofloxacin, trovafloxacin, and others), and lipopeptides (e.g., daptomycin, surfactin, and others).

Embodiment 73. The method of Embodiment 72, said subject is suffering from tuberculosis.

Embodiment 74. A method of treating cancer, the method comprising administering an effective amount of a compound as in Embodiments 1-48 to a subject suffering from cancer.

Embodiment 75. The method of Embodiment 76, wherein said subject is a human patient.

Embodiment 76. The method of Embodiment 76, wherein said cancer is selected from the group consisting of liver cancer, cholangiocarcinoma, osteosarcoma, melanoma, breast cancer, renal cancer, prostate cancer, gastric cancer, colorectal cancer, thyroid cancer, head and neck cancer, ovarian cancer, pancreatic cancer, neuronal cancer, lung cancer, uterine cancer, leukemia, and lymphoma.

The present invention is further illustrated by the following examples which should not be construed as further limiting. The contents of all references, patents and published patent applications cited throughout this application, as well as the Figures and the Appendix of sequences provided herein, are expressly incorporated herein by reference in their entirety.

### EXAMPLES

Exemplary analogs of pentamidine were designed and synthesized (see Table 1) using the synthesis methods described further below.

### General Information

¹H NMR spectra and ¹³C NMR spectra were recorded on a Varian 400 MHz or Bruker Avance III 500 MHz spectrometers. Spectra are referenced to residual chloroform (δ 7.26, ¹H), DMSO (δ 2.54, ¹H) or methanol (δ 3.34, ¹H) unless otherwise noted. Chemical shifts are reported in ppm (δ); multiplicities are indicated by s (singlet), d (doublet), t (triplet), q (quartet), quint (quintet), sext (sextet), m (multiplet) and br (broad). Coupling constants, J, are reported in Hertz. Silica gel chromatography was performed using a Teledyne Isco CombiFlash® Rf+ instrument using Hi-Purit Silica Flash Cartridges (National Chromatography Inco) or RediSep Rf Gold C18 Cartridges (Teledyne Isco). Analytical HPLC was performed on a Waters ACQUITY UPLC with a photodiode array detector using and a Waters ACQUITY BEH Shield RPC18 (2.1×50 mm, 1.7 μm) column. Analytical LCMS was performed on a Waters ACQUITY UPLC with a Waters 3100 mass detector. Chiral HPLC was performed on a Waters Alliance e2695 with a photodiode array detector using Daicel Chiralpak® AD-H, Chiralpak® IA, Chiralpak ® IB, Chiralpak® IC, Chiralcel® OD-H or Chiralcel® OJ-H columns. Optical rotations were obtained on a Jasco P-2000 digital polarimeter and are reported as [α]_{D}^{T} temperature (T), concentration (c=g/100 mL) and solvent. Commercially available reagents and solvents were used as received unless otherwise indicated.

TABLE 1
Exemplary Pentamidine Analogues

| Cmpd | | | IC₅₀ (μM) |
|---|---|---|---|
| No. | Structure | Name | [NCI-H69] |
| 1 | 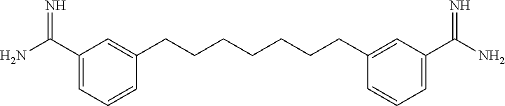 [SMILES: N=C(N)c1cccc(CCCCCCCc2cccc(C(=N)N)c2)c1] | 3,3′-(heptane-1,7- diyl)dibenzimidamide | 0.31 |
| 2 | 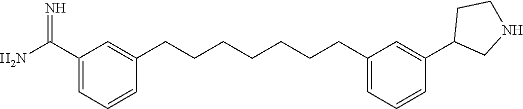 [SMILES: N=C(N)c1cccc(CCCCCCCc2cccc(C3CCNC3)c2)c1] | 3-(7-(3-(pyrrolidin-3- yl)phenyl)heptyl)benzimida- mide | 1.5 |
| 3 | 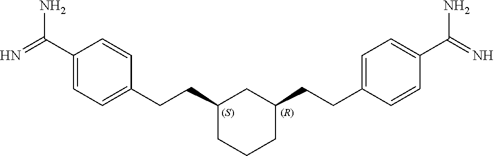 | 4,4′-(2,2′-((1R,3S)- cyclohexane-1,3- diyl)bis(ethane-2,1- diyl))dibenzimidamide | 0.8 |
| 4 | 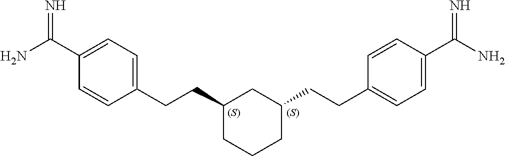 | 4,4′-(2,2′-((1R,3R)- cyclohexane-1,3- diyl)bis(ethane-2,1- diyl))dibenzimidamide | 0.75 |
| 5 | 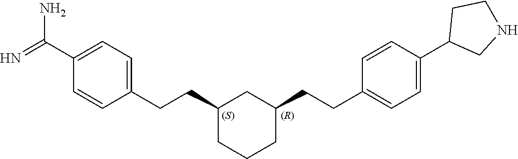 | 4-(2-((1S,3R)-3-(4- (pyrrolidin-3- yl)phenethyl)cyclohexyl)eth- yl)benzimidamide | 1.46 |
| 6 | 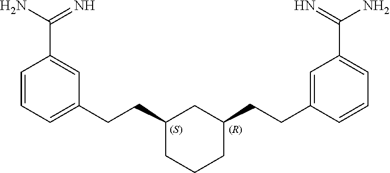 | 3,′-(((1R,3S)-cyclohexane- 1,3-diyl)bis(ethane-2,1- diyl))dibenzimidamide | 0.33 |
| 7 | 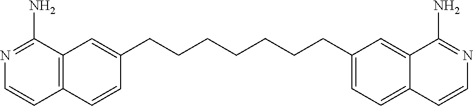 [SMILES: Nc1nccc2ccc(CCCCCCCc3ccc4ccnc(N)c4c3)cc12] | 7,7′-(heptane-1,7- diyl)bis(isoquinolin-1- amine) | 1.15 |
| 8 | 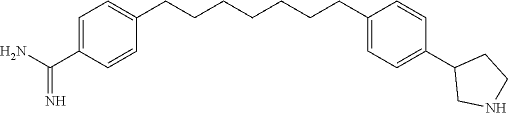 [SMILES: N=C(N)c1ccc(CCCCCCCc2ccc(C3CCNC3)cc2)cc1] | 4-(7-(4-(pyrrolidine-3- yl)phenyl)heptyl)benzimida- mide | 1.92 |

### Example 1

### Preparation of 3,3′-(heptane-1,7-diyl)dibenzimidamide

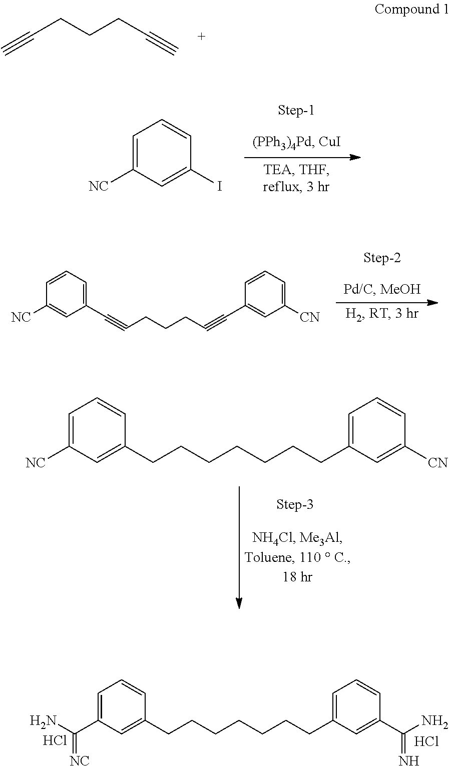

### Step 1

To a solution of hepta-1,6-diyne (0.8 g, 8.69 mmol, 1 eq.) in THF (40 mL) were added 3-iodobenzonitrile (5.96 g, 26.08 mmol, 3 eq.), triethylamine (4.4 mL, 26.08 mmol, 3 eq.) and CuI (0.16 g, 0.87 mmol, 0.1 eq.). Reaction mixture was deoxygenated by purging with N₂ for 20 minutes. To this mixture was added (PPh₃)₄Pd (0.5 g, 0.43 mmol, 0.05 eq.) and the mixture was again deoxygenated by purging with N₂ for 20 minutes. Reaction mixture was stirred under reflux for 3 h. Progress of reaction was monitored by TLC. Reaction mixture was cooled to room temperature (RT), filtered through celite-bed and washed with diethyl ether. Filtrate was evaporated under reduced pressure to afford crude material which was purified by Combi-Flash on silica gel using ethyl acetate-hexane (0-10%) as eluent to afford 3,3′-(hepta-1,6-diyne-1,7-diyl)dibenzonitrile (1.2 g, 47.05%).

LCMS: ₂₉₅ [M+1]⁺

### Step ₂

To a stirred suspension of Pd/C (0.06 g) in methanol under inert atmosphere was added 3,3′-(hepta-1,6-diyne-1,7-diyl)dibenzonitrile (0.4 g, 1.36 mmol, 1 eq.) and the resulting mixture was stirred under hydrogen atmosphere for ₃ h. Progress of reaction was monitored by TLC. After completion, reaction mixture was filtered through celite-bed and solvent was evaporated under reduced pressure to afford crude 3,3′-(heptane-1,7-diyl)dibenzonitrile (0.39 g, 95%) which was used in the next step without further purification.

LCMS: 303[M+1]⁺

### Step 3

To a suspension of ammonium chloride (0.28 g, 5.27 mmol, 8 eq.) in toluene (15 mL) at 0° C. was added trimethylaluminum (2.7 mL, 5.27 mmol, 8 eq.) dropwise. The mixture was allowed to stir at 0° C. for 10 minutes followed by stirring at room temperature for 15 minutes. To this solution was added 3,3′-(heptane-1,7-diyl)dibenzonitrile (0.2 g, 0.66 mmol, 1 eq.) and reaction mixture was allowed to stir at room temperature for 15 minutes followed by stirring at under reflux for 18 h. Reaction mixture was cooled to room temperature and then to 0° C. Reaction mixture was diluted with methanol (5 mL) and allowed to stir at RT for 30 minutes. Reaction mixture was diluted with 3M aq. HCl (50 mL) and washed with ethyl acetate (20 mL). Aqueous layer was basified with 5N NaOH (15 mL) and extracted with ethanol-ethyl acetate (20%, 3×50 mL). Combined organic layer was dried over anhydrous sodium sulfate. Removal of solvent afforded crude which was purified by reversed phase HPLC to afford 3,3′-(heptane-1,7-diyl)dibenzimidamide as free base. Solid was dissolved in 1.25 M HCl (5 mL), and the solution was concentrated under vacuum and lyophilized to afford 3,3′-(heptane-1,7-diyl)dibenzimidamide as di HCl salt (0.04 g, 18.8%).

LCMS: 337 [M+1]

¹H NMR (400 MHz, DMSO-d6) δ 9.30 (brs, 4H), 9.05 (brs, 4H), 7.45-7.59 (m, 4H), 7.60-7.70 (m, 4H), 1.52-1.65 (m, 4H), 1.20-1.40 (m, 6H).

### Example 2

### Preparation of 3-(7-(3-(pyrrolidin-3-yl)phenyl)heptyl)benzimidamide

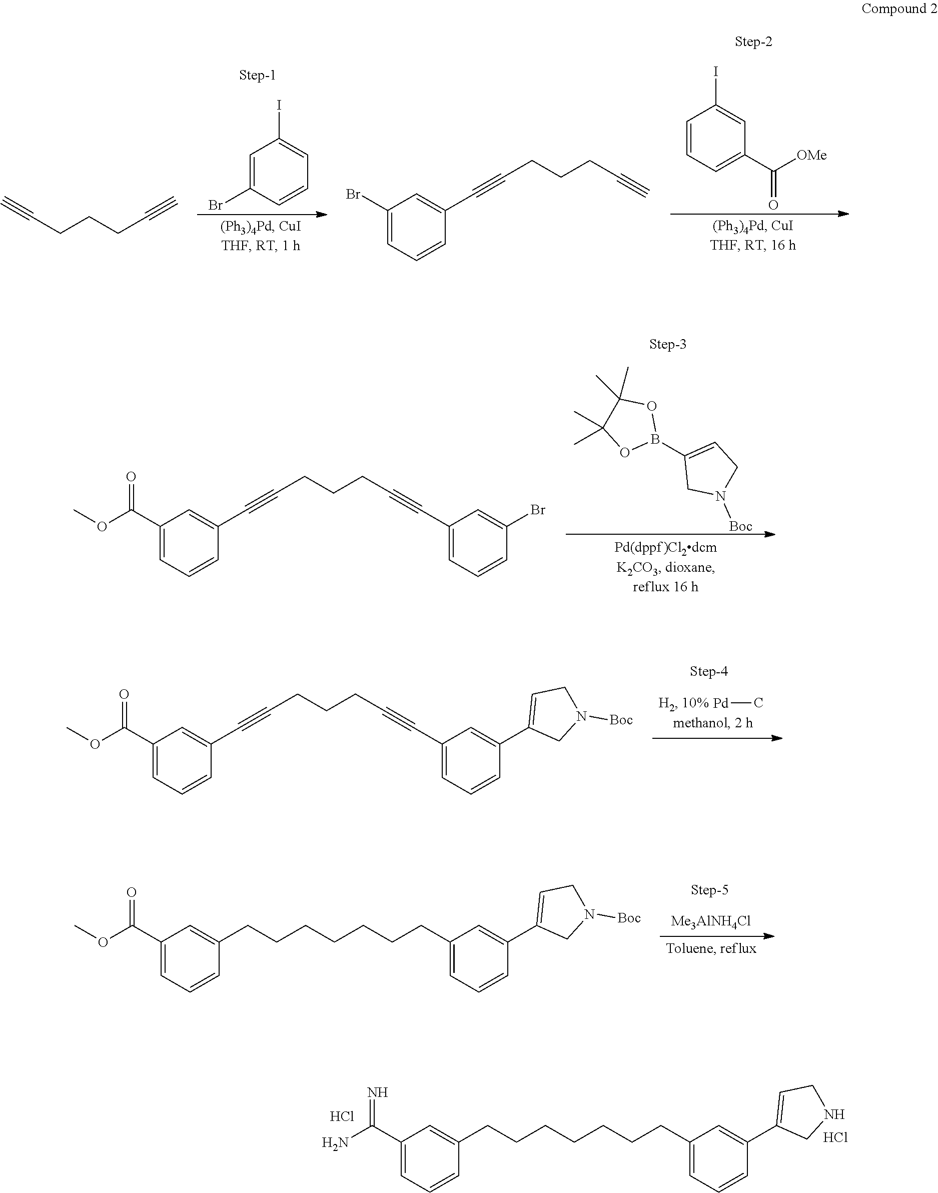

### Step 1

To stirred solution of hepta-1,6-diyne (0.65 g, 7.06 mmol, 1.0 eq.) in THF were added 1-bromo-3-iodobenzene (2.1 g, 7.77 mmol, 1.1 eq.) and diethylamine (2.1 mL, 21.18 mmol, 3.0 eq.). The reaction mixture was deoxygenated by purging with nitrogen for 15 minutes. To this mixture were added (Ph₃)₄Pd (81 mg, 0.071 mmol, 0.01 eq.) and CuI (53 mg, 0.28 mmol, 0.04 eq.) and the reaction was stirred at RT for 1 h. Progress of reaction was monitored by TLC and LCMS. After completion, reaction mixture was diluted with water (20 mL and extracted with ethyl acetate (3×20 mL). Combined organic layer was washed with brine (50 mL) and dried over anhydrous Na₂SO₄. Removal of solvent under reduced pressure gave crude material which was purified by Combi-Flash on silica gel using an ethyl acetate-hexane system as eluent to afford 1-bromo-3-(hepta-1,6-diyn-1-yl)benzene 1.5 g (88%).

LCMS: 328[M+1]⁺

### Step 2

To a stirred solution of 1-bromo-3-(hepta-1,6-diyn-1-yl)benzene (1.5 g, 6.06 mmol, 1.0 eq.) in THF were added methyl 3-iodobenzoate (2.06 g, 7.86 mmol, 1.3 eq.) and diethylamine (1.85 mL, 18.21 mmol 3.0 eq.). The reaction was deoxygenated by purging nitrogen for 15 minutes. To this mixture were added (Ph₃)₄Pd (0.35 g, 0.30 mmol 0.05 eq.) and CuI (58 mg, 0.30 mmol, 0.05 eq.). The reaction was allowed to stir under reflux for 16 h. The progress of reaction was monitored by TLC and LCMS. After completion, reaction mixture was diluted with water (20 mL) and extracted with ethyl acetate (3×20 mL). Combined organic layer was washed with brine, dried over anhydrous Na₂SO₄ and concentrated under reduced pressure to afford crude material which was purified by Combi-Flash on silica gel using an ethyl acetate-hexane system as eluent to afford methyl 3-(7-(3-bromophenyl)hepta-1,6-diyn-1-yl)benzoate (1 g, 43%).

LCMS: 381[M+1]⁺

### Step 3

To a stirred solution of methyl 3-(7-(3-bromophenyl)hepta-1,6-diyn-1-yl)benzoate (1.0 g, 2.63 mmol, 1.0 eq.) in a mixture of 1,4-dioxane and water (7:3, 20 mL) were added tert-butyl 3-(4,4,5,5-tetramethyl-1,3,2-dioxaborolan-2-yl)-2,5-dihydro-1H-pyrrole-lcarboxylate (0.76 g, 3.16 mmol, 1.2 eq.) and K₂CO₃ (1.0 g, 7.89 mmol, 3.0 eq.). The reaction mixture was deoxygenated by purging with nitrogen for 15 minutes. To this mixture was added PdCl₂(dppf)·dcm (0.45 g, 0.53 mmol, 0.2 eq.) and the reaction mixture was again deoxygenated by purging with nitrogen for 15 minutes. The reaction mixture was then allowed to stir under reflux for 16 h. Progress of reaction was monitored by TLC and LCMS. After completion, reaction mixture was cooled to RT, diluted with water (30 ml) and extracted with ethyl acetate (3×30 mL). Combined organic layer was washed with brine (50 mL) and dried over anhydrous sodium sulfate. Removal of solvent under reduced pressure afforded crude material which was purified by Combi-Flash on silica gel using an ethyl acetate-hexane system as eluent to afford tert-butyl 3-(3-(7-(3-(methoxycarbonyl)phenyl)hepta-1,6-diyn-1-yl)phenyl)-2,5-dihydro-1H-pyrrole-1-carboxylate (500 mg, 38%).

LCMS: 470[M+1]⁺

### Step 4

To a solution of tert-butyl 3-(3-(7-(3-(methoxycarbonyl)phenyl)hepta-1,6-diyn-1-yl)phenyl)-2,5-dihydro-1H-pyrrole-1-carboxylate (0.5 g, 1.07 mmol, 1.0 eq.) in methanol was added 10% Pd-C (0.5 g) and the reaction was stirred under hydrogen atmosphere for 2 h. Progress was monitored by TLC and ¹H NMR. Reaction mixture was filtered through celite-bed and bed was washed with methanol. Combined filtrate was concentrated under reduced pressure to afford tert-butyl 3-(3-(7-(3-(methoxycarbonyl) phenyl) heptyl)phenyl)pyrrolidine-1-carboxylate (350 mg, 70%) which was used in the next step without further purification.

LCMS: 480 [M+1]⁺

### Step 5

To a stirred suspension of NH₄Cl (0.312 g, 5.84 mmole, 8.0 eq.) in toluene (5 mL) at 0° C. was added a solution of 2M trimethylaluminum in toluene (2.92 mL, 5.84 mmol, 8.0 eq.) and the reaction mixture was allowed to stir at 0° C. for 10 minutes followed by stirring at RT for 15 minutes. To this solution was added tert-butyl 3-(3-(7-(3-(methoxycarbonyl)phenyl)heptyl)phenyl)pyrrolidine-1-carboxylate (350 mg, 0.75 mmol, 1.0 eq.) and the reaction mixture was allowed to stir at RT for 15 minutes followed by stirring under reflux for 18 h. The reaction mixture was cooled to RT, diluted with methanol (5 mL) under ice cold condition and then allowed to stir at RT for 30 minutes. The reaction mixture was diluted with 1N HCl (20 mL) and washed with ethyl acetate (20 mL). Aqueous layer was basified with 13N NaOH solution (15 mL) and extracted with 20% ethanol in ethyl acetate (3×20 mL). Combined organic layer was dried over anhydrous Na₂SO₄ and concentrated under vacuum to afford crude material which was purified by reversed phase HPLC to afford 3-(7-(3-(pyrrolidin-3-yl)phenyl)heptyl)benzimidamide as freebase. The solid was dissolved in 1.25 M HCl in ethanol (8 mL) and concentrated under reduced pressure to provide a solid which, after lyophilization, afforded 3-(7-(3-(pyrrolidin-3-yl)phenyl)heptyl)benzimidamide dihydrochloride (0.015 g, 20.46%).

LCMS: 363[M+1]⁺

¹H NMR (400 MHz, DMSO-d6) δ 9.45 (brs, 2H), 9.39 (brs, 2H), 9.12 (brs, 2H), 7.70-7.50 (m, 3H), 7.39-7.02 (m, 5H), 4.32 (brs, 1H), 3.70-2.98 (m, 4H), 2.70-2.40 (m, 6H), 1.98-1.80 (m, 2H), 1.61-1.50 (m, 4H), 1.40-1.20 (m, 4H).

### Example 3

### Preparation of 4,4′-(2,2′-((1R,3S)-cyclohexane-1,3-diyl)bis(ethane-2,1-diyl))dibenzimidamide

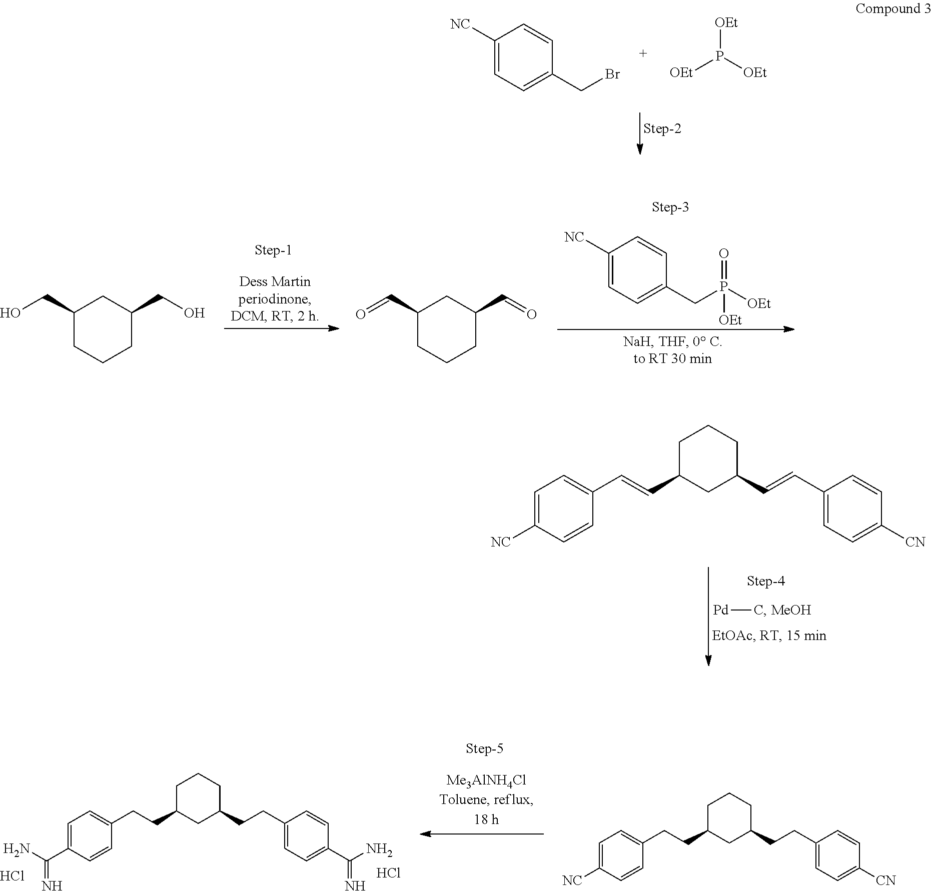

### Step 1

To a stirred suspension of cis-cyclohexane-1,3-diyldimethanol (0.4 g, 2.83 mmol, 1 eq.) in dichloromethane (5 mL) was added Dess-Martin periodinone (2.49 g, 5.87 mmol, 2.36 eq.) and the reaction mixture was allowed to stir at RT for 2 h. Progress of reaction was monitored by TLC and ¹H NMR. After completion, reaction mixture was filtered and washed with pentane (10 mL) and filtrate was evaporated under reduced pressure to afford crude which was triturated with pentane and filtered. Filtrate was evaporated to get cis-cyclohexane-1,3-dicarbaldehyde (0.4 g, crude) which was used in the next step without further purification.

### Step 2

A mixture of 4-(bromomethyl)benzonitrile (250 mg, 1.26 mmol, 1 eq.) and triethylphosphite (0.45 mL, 2.52 mmol, 2 eq.) was allowed to stir at 160° C. for 2 h. Progress of reaction was monitored by TLC. After completion, reaction mixture was cooled to RT to give crude diethyl 4-cyanobenzylphosphonate (600 mg) which was used in the next without purification.

### Step 3

To a stirred suspension of 4-cyanobenzylphosphonate (1.80 g, 7.14 mmol, 2.5 eq.) in THF (10 mL) was added NaH (0.17 g, 7.14 mmol, 2.5 eq.) at 0° C. and the reaction mixture was allowed to stir at the same temperature for 15 minutes. To this mixture was added a solution of cis-cyclohexane-1,3-dicarbaldehyde (0.4 g, 2.85 mmol, 1 eq.) in THF (5 mL) and the reaction was stirred at room temperature for lh. Progress of reaction was monitored by TLC. After completion, reaction mixture was diluted with water (30 mL) and extracted with ethyl acetate (3×40 mL). Combined organic layers was washed with brine (30 mL), dried over sodium sulfate and evaporated under reduced pressure to afford crude which was purified by Combi-Flash on silica gel using ethyl acetate-hexane system as eluent to afford 4,4′-(1E,1′E)-2,2′4(1R,3S)-cyclohexane-1,3-diyl)bis(ethene-2,1-diyl)dibenzonitrile (0.4 g, 41.36%)

LCMS: 339 [M+1]⁺

### Step 4

To a stirred suspension of 4′-(1E,1E)-2,2′4(1R,3S)-cyclohexane-1,3-diyl)bis(ethene-2,1-diyl)dibenzonitrile (0.4 g,1.18 mmol) in methanol (10 mL) and ethyl acetate (5 mL) was added Pd-C (120 mg). The reaction mixture was allowed to stir at RT under hydrogen atmosphere for 15 min. Progress of reaction was monitored by 1 H NMR. After completion, reaction mixture was filtered through a celite-bed, bed washed with ethyl acetate (20 mL) and filtrate was evaporated under reduced pressure to afford crude which was purified by Combi-Flash on silica gel using an ethyl acetate-hexane system as eluent to afford 4,4′-(2,2′-(1R,3S)-cyclohexane-1,3-diyl)bis(ethane-2,1-diyl))dibenzonitrile (0.3 g, 74.25%).

LCMS: 343.4 [M+1]⁺

### Step 5

To a stirred suspension of NH₄Cl (0.47 g, 8.74 mmol, 10 eq.) in toluene (10 mL) at 0° C. was added 2M solution of trimethylaluminum in toluene (4.3 mL, 8.74 mmol, 10 eq.). The reaction mixture was allowed to stir at 0° C. for 10 minutes followed by stirring at RT for minutes. To this solution was added 4,4′-(2,2′-((1R,3S)-cyclohexane-1,3-diyl)bis(ethane-2,1-diyl))dibenzonitrile (0.3 g, 0.847 mmol, 1.0 eq.) and reaction mixture was allowed to stir at room temperature for 15 min. The reaction mixture was then stirred under reflux for 18 h. The reaction mixture was cooled to RT and to it was added methanol (5 mL) under ice cooled condition and the reaction mixture was allowed to stir at RT for 30 minutes. The reaction mixture was diluted with 1N HCl (30 mL) and washed with ethyl acetate (20 mL). Aqueous layer was basified with 3N NaOH solution (20 mL) and extracted with 20% ethanol in ethyl acetate (3×60 mL). Combined organic layer was dried over anhydrous Na₂SO₄ and concentrated under vacuum to afford crude material (0.3 g) which was purified by reversed phase HPLC to afford 4,4′-(2,2′-((1R,3S)-cyclohexane-1,3-diyl)bis(ethane-2,1-diyl))dibenzimidamide as a freebase. The solid obtained was dissolved in 1.25 M HC1 in ethanol (10 mL), concentrated under reduced pressure to yield a solid material which after lyophilization afforded 4,4′-(2,2′4(1R,3S)-cyclohexane-1,3-diyl)bis(ethane-2,1-diyl))dibenzimidamide as dihydrochloride salt (0.152 g, 47.11%).

LCMS: 377.3 [M+1]⁺

¹H NMR (400 MHz, DMSO-d6) δ 9.30 (brs, 4H), 9.08 (brs, 4H), 7.75 (d, 4H), 7.42 (d, 4H), 2.77-2.60 (m, 4H), 1.85-1.61 (m, 6H), 1.30-1.05 (m, 4H), 0.95-0.55 (m, 4H).

### Example 4

### Preparation of 4,4′-(2,2′-((1R,3R)-cyclohexane-1,3-diyl)bis(ethane-2,1-diyl))dibenzimidamide

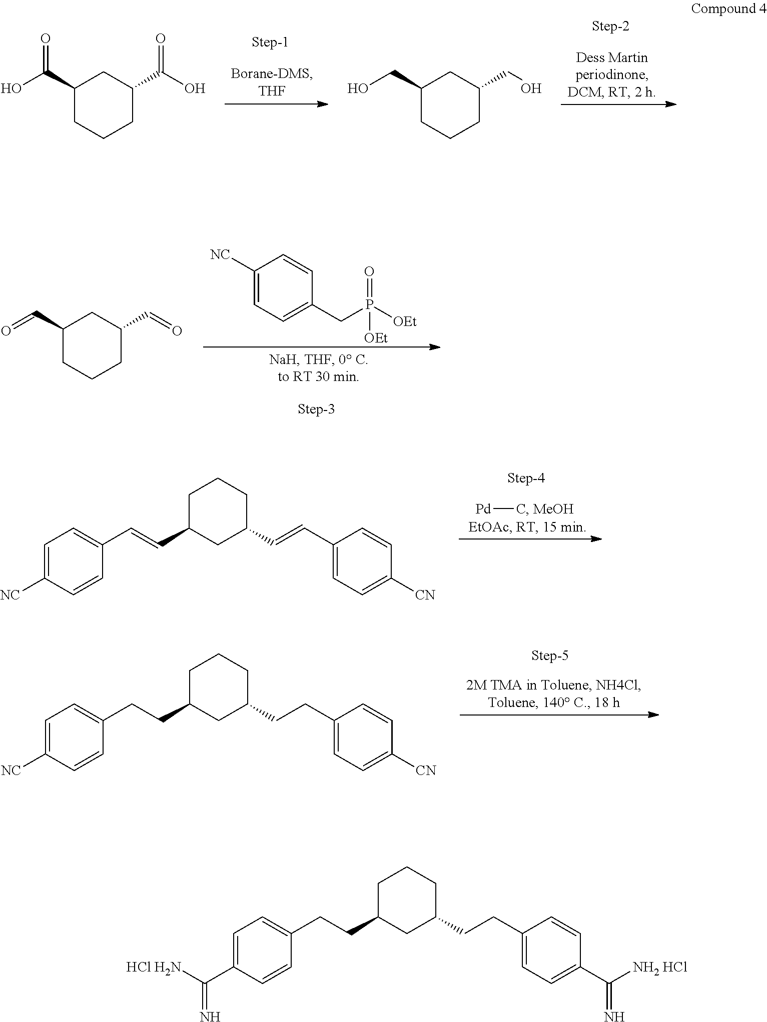

### Step 1

To a stirred solution of (1R,3R)-cyclohexane-1,3-dicarboxylic acid (1 g, 5.80 mmol, 1 eq.) in THF (10 mL) was added Borane-DMS (1.6 mL, 17.40 mmol, 3.0 eq.) at 0° C. and the reaction mixture was allowed to stir at RT for 2 h. Progress of reaction was monitored by TLC and ¹H NMR. After completion, reaction mixture was quenched with 1N HCl (100 mL) and extracted with extracted with ethyl acetate (3×40 mL). Combined organic layers were washed with brine (30 mL), dried over sodium sulfate and evaporated under reduced pressure to afford crude material which was purified by Combi-Flash on silica gel using ethyl acetate-hexane system as eluent to afford (1R,3R)-cyclohexane-1,3-diyldimethanol (700 mg, 83.63%)

### Step 2

To a stirred suspension of (1R,3R)-cyclohexane-1,3-diyldimethanol (300 mg, 2.08 mmol, 1 eq.) in dichloromethane (10 mL) was added Dess-Martin periodinone (2.70 g, 4.16 mmol, 3.0 eq.) and the reaction mixture was allowed to stir at RT for 2 h. Progress of reaction was monitored by TLC and ¹H NMR. After completion, reaction mixture was filtered and washed with pentane (10 mL) and filtrate was evaporated under reduced pressure to afford crude which was triturated with pentane (2×10 mL) and filtered. Filtrate was evaporated to obtain pure (1R,3R)-cyclohexane-1,3-dicarbaldehyde (190 mg) which was used in the next step without further purification.

### Step 3

To a stirred suspension of 4-cyanobenzylphosphonate (1. g, 4.07 mmol, 3.0 eq.) in THF (10 mL) was added 1M solution of potassium tert-butoxide (3.9 mL 3.91 mmol, 2.9 eq.) at 0° C. and reaction mixture was allowed to stir at 0° C. for 15 minutes. To this mixture was added a solution of (1R,3R)-cyclohexane-1,3-dicarbaldehyde (190 mg, 1.35 mmol, 1 eq.) in THF (5 mL) and the reaction was stirred at room temperature for 1 h. Progress of reaction was monitored by TLC. After completion, reaction mixture was diluted with water (30 mL) and extracted with ethyl acetate (3×40 mL). Combined organic layers was washed with brine (30 mL), dried over sodium sulfate and evaporated under reduced pressure to afford crude which was purified by Combi-Flash on silica gel using ethyl acetate-hexane system as eluent to afford 4,4′-[(1S,3S)-cyclohexane-1,3-diyldi(E)ethene-2,1-diyl]dibenzonitrile (0.2 g, 79.05%).

LCMS: 339 [M+1]⁺

### Step 4

To a stirred suspension of 4,4′-[(1S,3S)-cyclohexane-1,3-diyldi(E)ethene-2,1-diyl]dibenzonitrile (200 mg, 0.59 mmol) in methanol (10 mL)) was added Pd-C (40 mg). The reaction mixture was allowed to stir at RT under hydrogen atmosphere for 30 minutes. Progress of reaction was monitored by TLC. After completion, reaction mixture was filtered through celite-bed and bed washed with methanol (20 mL) and filtrate was evaporated under reduced pressure to afford 4,4′-(2,2′-((1R,3R)-cyclohexane-1,3-diyl)bis(ethane-2,1-diyl))dibenzonitrile (200 mg, 99.00%).

LCMS: 343.4 [M+1]⁺

### Step 5

To a stirred suspension of NH₄Cl (250 mg, 4.67 mmol, 8.0 eq.) in toluene (10 mL) at 0° C. was added 2M solution of trimethylaluminum (in toluene) (5.0 mL, 4.67 mmol, 8 eq.). The reaction mixture was allowed to stir at 0° C. for 10 minutes followed by stirring at RT for 15 minutes. To this solution was added 4,4′-(2,2′-((1R,3R)-cyclohexane-1,3-diyl)bis(ethane-2,1-diyl))dibenzonitrile (200 mg, 0.58 mmol, 1.0 eq.) and reaction mixture was allowed to stir at room temperature for 15 min. The reaction mixture was then stirred under reflux for 18 h. The reaction mixture was cooled to RT and to it was added methanol (5 mL) under ice cooled condition and reaction mixture was allowed to stir at RT for 30 minutes. The reaction mixture was diluted with 1N HCl (30 mL) and washed with ethyl acetate (20 mL). Aqueous layer was basified with 1N NaOH solution (20 mL) and extracted with ethanol-ethyl acetate (20%, 3×60 mL). The separated organic layer were dried over anhydrous Na₂SO₄ and concentrated under vacuum to afford crude (0.3 g) which was purified by reversed phase HPLC to 4,4′-(2,2′-((1R,3R)-cyclohexane-1,3-diyl)bis(ethane-2,1-diyl))dibenzimidamide. Solid was dissolved in 1.25 M HCl in ethanol (10 mL), solvent was evaporated under reduced pressure to get solid which after lyophilisation afforded 4,4′-(2,2′-((1R,3R)-cyclohexane-1,3-diyl)bis(ethane-2,1-diyl))dibenzimidamide as dihydrochloride salt (40 mg, 15.32%).

LCMS: 377.3 [M+1]⁺

¹H NMR (400 MHz, DMSO-d6) δ 9.30 (brs, 4H), 9.08 (brs, 4H), 7.75 (d, 4H), 7.42 (d, 4H), 2.77-2.60 (m, 4H), 1.85-1.61 (m, 6H), 1.30-1.05 (m, 4H), 0.95-0.55 (m, 4H).

### Example 5

### Preparation of 4-(2-((1S,3R)-3-(4-(pyrrolidin-3-yl)phenethyl)cyclohexyl)ethyl)benzimidamide

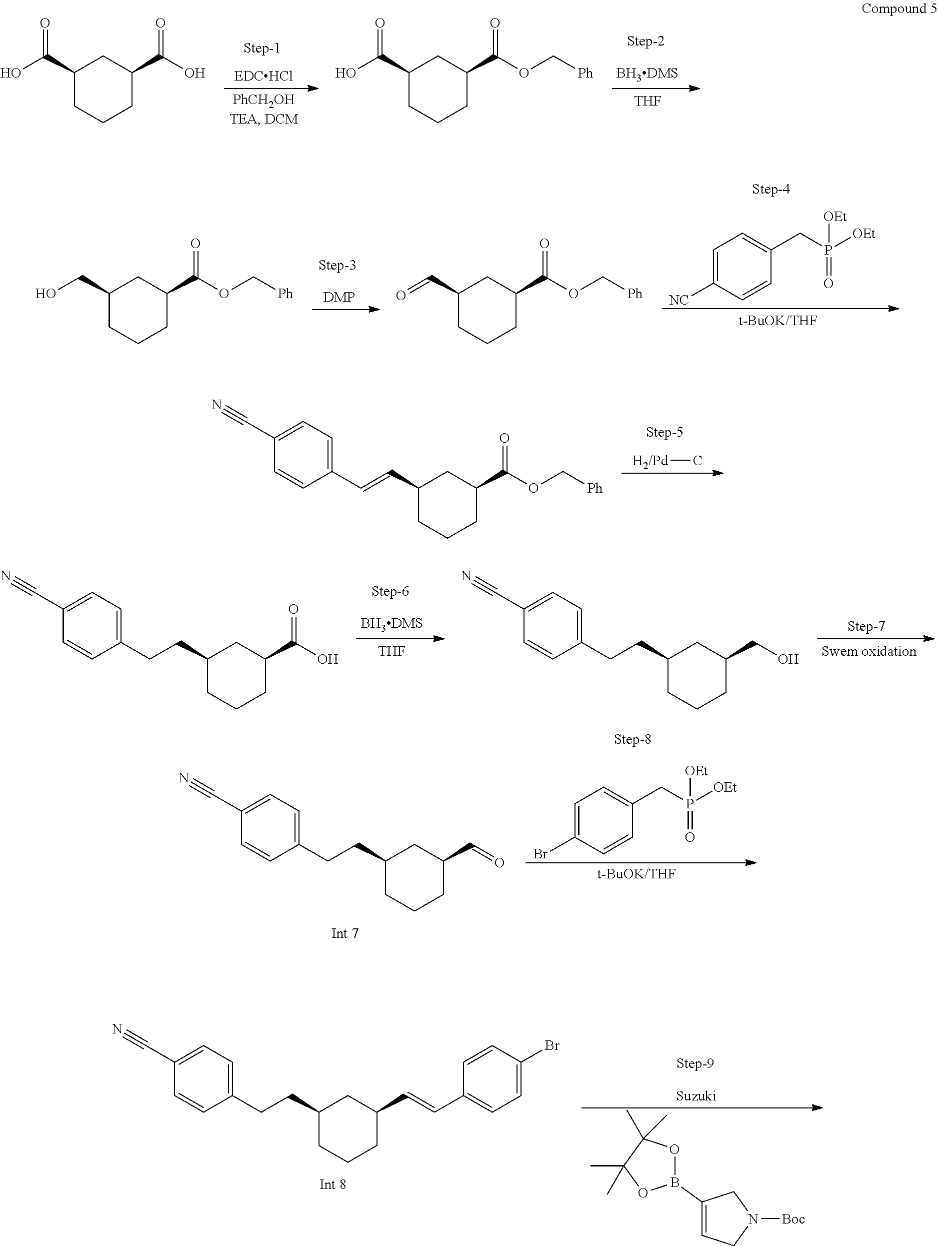

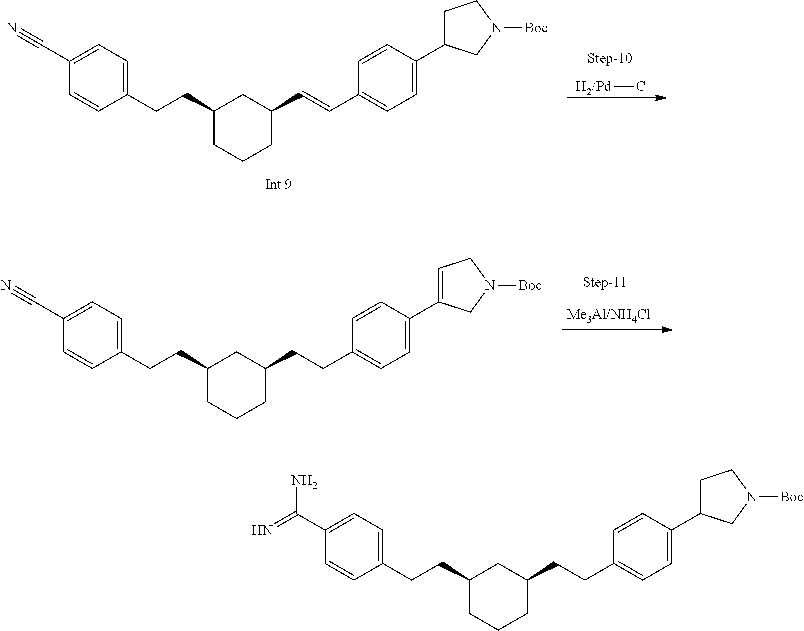

### Step 1

To a stirred solution of (1R,3S)-cyclohexane-1,3-dicarboxylic acid (2 g, 11.6 mmol, 1 eq.), benzyl alcohol (1.25 g, 11.6 mmol, 1 eq.) and EDC.HC1 (2.22 g, 11.6 mmol, 1 eq.) in dichloromethane (100 mL) was added triethylamine (3.24 mL, 23.2 mmol, 2 eq.) and the reaction mixture was allowed to stir at RT for 16 h. Reaction mixture was diluted with dichloromethane (100 mL), washed with 1N aq. HCl solution (2×50 mL) followed by brine (50 mL) and dried over anhydrous sodium sulfate. Removal of solvent under reduced pressure afforded crude (1R,3S)-3-(benzyloxycarbonyl)cyclohexanecarboxylic acid (3 g, 98%) which was used in next step without further purification.

### Step 2

To a stirred solution of (1R,3S)-3-(benzyloxycarbonyl)cyclohexanecarboxylic acid (3 g, 11.4 mmol, 1 eq.) in THF (200 mL) at 0° C. was added borane.dimethylsulfide complex (3.25 mL, 34.3 mmol, 3 eq.) dropwise and reaction the mixture was allowed to stir at 0° C. 3 h. Progress of reaction was monitored by ¹H NMR. After completion, reaction was quenched with 1 N aq HCl (70 mL) and extracted with ethyl acetate (3×100 mL). Combined organic layer was washed with brine (50 mL) and dried over anhydrous sodium sulfate. Removal of solvent under reduced pressure afforded crude (1S,3R)-benzyl 3-(hydroxymethyl)cyclohexanecarboxylate (3 g) which was used in next step without further purification.

### Step 3

To a stirred solution of (1S,3R)-benzyl 3-(hydroxymethyl)cyclohexanecarboxylate (1.8 g, 7.26 mmol, 1 eq.) in dichloromethane (40 mL) at RT was added Dess-Martin periodinane (3.39 g, 7.99 mmol, 1.1 eq.) portion wise and reaction mixture was allowed to stir at RT for 2 h. Progress of reaction was monitored by TLC and ¹H NMR. After completion, reaction mixture was then directly purified by Combi-Flash on silica gel using ethyl acetate-hexane system as eluent to afford (1S,3R)-benzyl 3-formylcyclohexanecarboxylate (1.3 g, 73%).

### Step 4

To a stirred solution of diethyl 4-cyanobenzylphosphonate (1.18 g, 4.66 mmol, 1.5 eq.) in THF (10 mL) at 0° C. was added a solution 1M potassium tert-butoxide in THF (4.35 mL, 4.35 mmol, 1.4 eq.) dropwise and the resulting mixture was allowed to stir at the same temperature for 15 minutes. To this solution was added a solution of (1S,3R)-benzyl 3-formylcyclohexanecarboxylate (0.766 g, 3.11 mmol, 1 eq.) in THF (5 mL) and the reaction mixture was allowed to stir at RT for 45 minutes. Progress of reaction was monitored by TLC. After completion, reaction mixture was diluted with aq. ammonium chloride solution (40 mL) and extracted with ethyl acetate (3×50 mL). The combined organic layer was washed with brine (20 mL) and dried over anhydrous sodium sulfate. Removal of solvent under reduced pressure gave crude material which was purified by Combi-Flash on silica gel using an ethyl acetate-hexane system as eluent to afford (1S,3R)-benzyl 3-(4-cyanostyryl)cyclohexanecarboxylate (0.7 g , 66.1%).

### Step 5

To a solution of (1S,3R)-benzyl 3-(4-cyanostyryl)cyclohexanecarboxylate (708 mg, 2.0 mmol, 1 eq.) in methanol (25 mL) was added Pd-C (41 mg) and the reaction mixture was allowed to stir at RT under hydrogen atmosphere for 25 minutes. Progress of reaction was monitored by TLC and ¹H NMR. After completion, reaction mixture was filtered through celite-bed and bed was washed with methanol (20 mL). Filtrate was concentrated under reduced pressure to afford (1S,3S)-3-(4-cyanophenethyl)cyclohexanecarboxylic acid (571 mg) which was used in next step without further purification.

### Step 6

To a stirred solution of (1S,3S)-3-(4-cyanophenethyl)cyclohexanecarboxylic acid (0.640 g, 2.4 mmol, 1 eq.) in THF (30 mL) at 0° C. was added borane.dimethylsulfide complex (1.1 mL, 11.9 mmol, 4.79 eq.) portion wise and the reaction mixture was allowed to stir at 0° C. for 3 h. Progress of reaction was monitored by TLC. After completion, reaction was quenched with 1 N aq HCl solution (50 mL) and extracted with ethyl acetate (3×100 mL). Combined organic layer was washed with brine (50 mL), dried over anhydrous sodium sulfate and concentrated under reduced pressure to provide crude material which was purified by Combi-Flash on silica gel using an ethyl acetate-hexane system as eluent to afford 4-(2-((1S,3S)-3-(hydroxymethyl)cyclohexyl)ethyl)benzonitrile (345 mg , 57%).

### Step 7

To a stirred solution of oxalyl chloride (0.6 mL, 6.94 mmol, 4.9 eq.) in dichloromethane (5 mL) at −78° C. was added a solution of DMSO (0.8 mL, 11.2 mmol, 7.9 eq.) in dichloromethane (5 mL) and reaction mixture was allowed to stir at −78° C. for 30 minutes. To this solution was added a solution of 4-(2-((1S,3S)-3-(hydroxymethyl)cyclohexyl)ethyl)benzonitrile (0.345 g, 1.41 mmol, 1 eq.) in dichloromethane (5 mL) and reaction mixture was allowed to stir at −78° C. for 45 minutes. To the reaction mixture was added a solution of trimethylamine (2 mL, 14.3 mmol, 10.1 eq.) in dichloromethane (5 mL) and the reaction mixture was allowed to stir at −78° C. for 30 minutes followed by stirring at RT for 20 minutes. The reaction mixture was diluted with water (50 mL) and extracted with dichloromethane (3×50 mL). Combined organic layer was washed with brine (50 mL) and dried over anhydrous sodium sulfate. Removal of solvent under reduced pressure afforded crude 4-(2-((1S,3S)-3-formylcyclohexyl)ethyl)benzonitrile (0.320 g, 93%) which was used in the next step without purification.

### Step 8

To the stirred solution of diethyl 4-bromobenzylphosphonate (0.610 g, 1.98 mmol, 1.5 eq.) in THF (10 mL) at 0° C. was added 1M solution of potassium tert-butoxide in THF (1.98 mL, 1.98 mmol, 1.5 eq.) dropwise and the reaction mixture was stirred at the same temperature for 15 minutes. To this solution was added a solution of 4-(2-((1S,3S)-3-formylcyclohexyl)ethyl)benzonitrile (0.320 g, 1.32 mmol, 1 eq.) in THF (5 mL) and the reaction mixture was allowed to stir at RT for 45 minutes. Progress of reaction was monitored by TLC. After completion, reaction was quenched with aq. ammonium chloride solution (40 mL) and extracted with ethyl acetate (3×50 mL). Combined organic layer was washed with brine (40 mL), dried over anhydrous sodium sulfate and concentrated under reduced pressure to obtain crude material which was purified by Combi-Flash on silica gel using ethyl acetate-hexane system as eluent to afford 4-(2-((1S,3S)-3-(4-bromostyryl)cyclohexyl)ethyl) benzonitrile (0.255 g, 48.8%).

### Step 9

To a solution of 4-(2-((1S,3S)-3-(4-bromostyryl)cyclohexyl)ethyl)benzonitrile (255 mg, 0.64 mmol, 1 eq.) and tert-butyl 3-(4,4,5,5-tetramethyl-1,3,2-dioxaborolan-2-yl)-2,5-dihydro-1H-pyrrole-1-carboxylate (209 mg, 0.71 mmol, 1.1 eq.) in a solution of water (1 mL) in dioxane (12 mL) was added potassium carbonate (491 mg, 3.55 mmol, 5.5 eq.) and the reaction mixture was deoxygenated using nitrogen gas for 10 minutes. To this solution was added PdCl₂·dppf·CH₂Cl₂ (52 mg, 0.06 mmol, 0.1 eq.) and the reaction mixture was again deoxygenated using nitrogen gas for 10 minutes. The reaction mixture was allowed to stir at 85° C. for 2 h. Progress of reaction was monitored by TLC and NMR. Reaction mixture was cooled to RT, diluted with ethyl acetate (100 mL) and filtered through a celite-bed. The filtrate was dried over anhydrous sodium sulfate. Removal of solvent gave a crude oily product which was purified by silica gel chromatography on Combi-Flash to afford tert-butyl 3-(4-((E)-24(1S,3S)-3-(4-cyanophenethyl)cyclohexyl)vinyl)phenyl)-2,5-dihydro-1H-pyrrole-1-carboxylate (260 mg, 83.3%).

### Step 10

To a solution of tert-butyl 3-(4-((E)-24(1S,3S)-3-(4-cyanophenethyl)cyclohexyl)vinyl)phenyl)-2,5-dihydro-1H-pyrrole-1-carboxylate (200 mg, mmol, 1 eq.) in methanol (125 mL) was added Pd-C (80 mg) and the reaction mixture was allowed to stir at RT under hydrogen atmosphere for 25 minutes. Progress of reaction was monitored by TLC and ¹H NMR. After completion, reaction mixture was filtered through celite-bed and bed was washed with methanol (20 mL). Filtrate was concentrated under reduced pressure to afford desired product as crude which was purified by silica gel chromatography on Combi-Flash to afford tert-butyl 3-(4-(2-((1R,3S)-3-(4-cyanophenethyl)cyclohexyl)ethyl)phenyl)pyrrolidine-1-carboxylate (190 mg, 95%).

### Step 11

To a suspension of NH₄Cl (184 mg, 3.45 mmol, 8 eq.) in toluene (5 mL) at 0° C. was added 2M Me₃Al in toluene (1.72 mL, 3.45 mmol, 8 eq.) dropwise and the mixture was allowed to stir at the same temperature for 15 minutes. The mixture was brought to RT and allowed to stir for additional 10 minutes. To this mixture was added a solution of tert-butyl 3-(4-(2-((1R,3S)-3-(4-cyanophenethyl)cyclohexyl)ethyl)phenyl)pyrrolidine-1-carboxylate (210 mg, 0.431 mmol, 1 eq.) in toluene (5 mL) and reaction mixture was allowed to stir at RT for another 10 minutes and then allowed to stir at 120° C. for 18 h. Reaction mixture was cooled to RT, diluted with methanol (5 mL) and allowed to stir at RT for 15 minutes. Reaction mixture was diluted with 3M aq. HCl (15 mL) and washed with ethyl acetate (30 mL). Aqueous layer was basified with 3M Aq. NaOH solution and extracted with 20% ethanol-ethyl acetate solution (3×50 mL). Combined organic layer was dried over sodium sulfate and concentrated under vacuum to afford crude material which was purified by reversed phase HPLC to afford desired product as free base. The solid was dissolved in 1.25 M HCl in ethanol (3 mL) and concentrated to obtain a solid which was lyophilized to afford 4-(2-((1S,3R)-3-(4-(pyrrolidin-3-yl)phenethyl)cyclohexyl)ethyl)benzimidamide as diHCl salt (9 mg , 4.3%).

LCMS 404.4 [M+1]⁺

¹H NMR (400 MHz, CD₃OD) δ 7.75 (d, 2H), 7.45 (d, 2H), 7.22 (d, 2H), 7.18 (d, 2H), 3.71-3.10 (m, 6H), 2.80-2.70 (m, 1H), 2.65-2.58 (m, 1H), 2.47-2.38 (m, 2H), 2.18-1.75 (m, 4H), 1.60-1.18 (m, 8H), 1.00-0.60 (m, 4H).

### Example 6

### Preparation of 3,3′-(((1R,3S)-cyclohexane-1,3-diyl)bis(ethane-2,1-diyl))dibenzimidamide

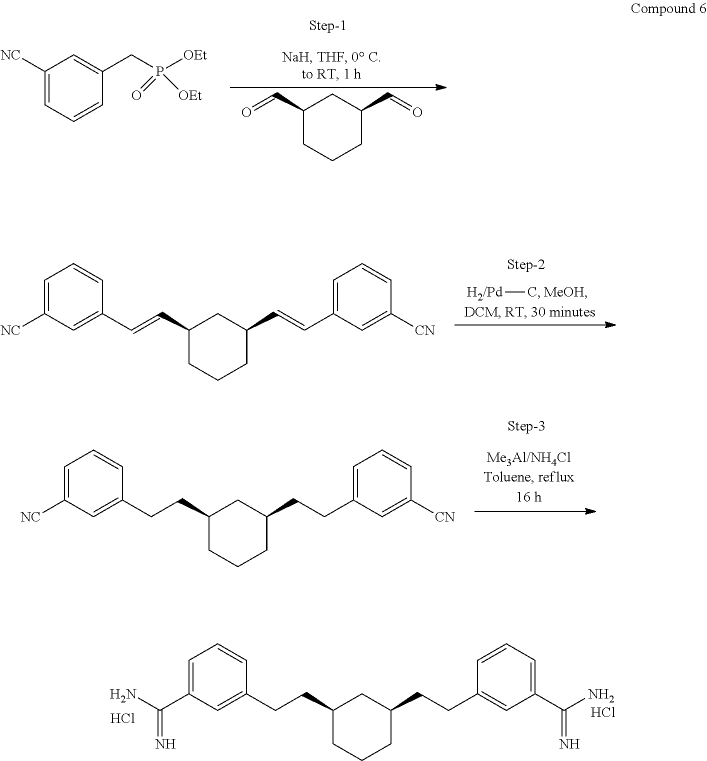

### Step 1

To a stirred suspension of diethyl (3-cyanobenzyl)phosphonate (976 mg, 3.85 mmol, 3.0 eq.) in THF (10 mL) was added NaH (154 mg, 3.85 mmol, 3.0 eq.) at 0° C. and the reaction mixture was allowed to stir for 15 minutes. To this mixture was added a solution of (1R,3S)-cyclohexane-1,3-dicarbaldehyde (180 mg, 1.28 mmol, 1.0 eq.) in THF (5 mL) and the reaction was stirred at room temperature for 1 h. Progress of reaction was monitored by TLC. After completion, reaction mixture was diluted with water (30 mL) and extracted with ethyl acetate (3×50 mL). Combined organic layers was washed with brine (30 mL), dried over sodium sulfate and evaporated under reduced pressure to afford crude which was purified by Combi-Flash on silica gel using ethyl acetate-hexane system as eluent to afford 3,3′4(1E,1′E)-((1R,3S)-cyclohexane-1,3-diyl)bis(ethene-2,1-diyl))dibenzonitrile (90 mg, 20.1%)

### Step 2

To a stirred solution of 3,3′-((1E,1′E)-((1R,3S)-cyclohexane-1,3-diyl)bis(ethene-2,1-diyl))dibenzonitrile (145 mg, 0.42 mmol, 1.0 eq.) in a solution of DCM (5 mL) and methanol (40 mL) was added Pd-C (30 mg) and the reaction mixture was allowed to stir at RT under hydrogen atmosphere for 30 minutes. Progress of reaction was monitored by TLC and ¹H NMR. After completion, reaction mixture was filtered through celite-bed and bed washed with methanol (50 mL) and filtrate was evaporated under reduced pressure to afford crude which was purified by Combi-Flash on silica gel using ethyl acetate-hexane system as eluent to afford 3,3′-(((1R,3S)-cyclohexane-1,3-diyl)bis(ethane-2,1-diyl))dibenzonitrile (130 mg, 89.9%).

### Step 5

To a stirred suspension of NH₄Cl (162.5 mg, 3.03 mmol, 8.0 eq.) in toluene (5 mL) at 0° C. was added 2M solution of trimethylaluminum in toluene (1.51 mL, 3.03 mmol, 8.0 eq.). The reaction mixture was allowed to stir at 0° C. for 10 minutes followed by stirring at RT for 15 minutes. To this solution was added 3,3′-(((1R,3S)-cyclohexane-1,3-diyl)bis(ethane-2,1-diyl))dibenzonitrile (130 mg, 0.37 mmol, 1.0 eq.) and reaction mixture was allowed to stir at room temperature for 15 min. The reaction mixture was then stirred under reflux for 16 h. The reaction mixture was cooled to RT and to it was added methanol (5 mL) under ice cooled condition and reaction mixture was allowed to stir at RT for 30 minutes. The reaction mixture was diluted with 1N HCl (15 mL) and washed with ethyl acetate (20 mL). Aqueous layer was basified with 1N NaOH solution (20 mL) and extracted with 20% ethanol in ethyl acetate (5×40 mL). The separated organic layer were dried over anhydrous Na₂SO₄ and concentrated under vacuum to afford crude (130 mg) which was purified by reversed phase HPLC to afford 3,3′-(((1R,3S)-cyclohexane-1,3-diyl)bis(ethane-2,1-diyl))dibenzimidamide as a freebase. Solid was dissolved in 1.25 M HCl in ethanol (5 mL), solvent was evaporated under reduced pressure to provide a solid which after lyophilisation afforded 3,3′-(((1R,3S)-cyclohexane-1,3-diyl)bis(ethane-2,1-diyl))dibenzimidamide as dihydrochloride salt (30 mg, 15%).

LCMS: 377.4 [M+1]⁺

¹H NMR (400 MHz, DMSO-d6) δ 9.38 (brs, 4H), 9.18 (brs, 4H), 7.75 (d, 4H), 7.42 (d, 4H), 2.77-2.60 (m, 4H), 1.85-1.61 (m, 6H), 1.30-1.05 (m, 4H), 0.95-0.55 (m, 4H).

### Example 7. In Vitro Evaluation of Cytotoxicity of Compounds 1-6

The experiment tested pentamidine, Compounds 1-6 in full growth media for 8 days. NCI H69 cells were initially treated with the test compounds on Day 0, and the cells were then replenished with fresh compound dilutions on Day 3. Pentamidine and Compound 1 were tested at 9 concentration points: 100 μM, 33.33 μM, 11.11 μM, 3.70 μM, 1.23 μM, μM, 0.14 μM, 0.05 μM, and 0.02 μM (final DMSO concentration=0.5%). The raw data values from the CellTiter-Glo™ cell viability assay expressed in relative luminescence units were normalized to the vehicle for each individual plate, and any reduction in luminescence indicated a decrease in viability (%). The data was analyzed in GraphPad PRISM using a non-linear sigmoidal plot with variable slope (asymmetric four-point linear regression), and an IC₅₀ value for each compound was generated based on the normalized dose-response curves and shown in Table 1.

### Example 8. In Vitro Antibacterial Activity

To evaluate the antibacterial properties of the compounds of the present inventions, Compound 3 was tested alone and in combination of rifampicin by using 17 clinically relevant bacterial strains in checkerboard assays. The 17 test strains/isolates were: (1) Serratia marcescens (ATCC 13880); (2) Salmonella typhimurium (ATCC 13311); (3) Salmonella choleraesuis (ATCC 10708); (4) Acinetobacter baumannii (ATCC BAA-1605); (5) A. baumannii (ATCC 17978); (6) A. baumannii (FDA-CDC AR-BANK#0088); (7) Citrobacter freundii (ATCC 8090); (8) Pseudomonas aeruginosa (BCCM 27647); (9) P. aeruginosa (BCCM 27648); (10) Escherichia coli (ATCC 25922); (11) E. coli (ATCC 10536); (12) Stenotrophomonas maltophilia (ATCC 13637); (13) Enterobacter cloacae (ATCC BAA-1143); (14) E. cloacae (ATCC 13047); (15) E. aerogenes (ATCC 13048); (16) Klebsiella pneumoniae (NCTC 13438); and (17) Klebsiella pneumoniae (FDA-CDC AR-BANK#0160). These bacterial strains were obtained from the American Type Culture Collection (ATCC, Rockville, MD, USA); Belgian Coordinated Collections of Microorganisms; The Culture Collections of Public Health England, UK; and FDA-CDC anticimrobial Resistance Isolate Bank, USA, and cryopreserved as single-use frozen working stock cultures which were stored at −80° C.

The 17 test strains were grown on nutrient agar or tryptic soy agar (TSA) medium as below. Colonies were suspended in phosphate buffered saline (PBS) pH 7.4. The absorbance of the bacterial suspension was measured with a spectrophotometer at OD₆₂₀ₙₘ, and then the suspension was adjusted to 1-2×10⁸ CFU/mL in PBS. The adjusted suspension was further diluted 1:500 in cation-adjusted Mueller-Hinton Broth. Each well contained approximately 5×10⁵ CFU/mL. The actual microbial count in the assay ranged from 2˜8×10⁵ CFU/mL as determined from dilution plating. The testing inoculum was prepared within minutes before adding to wells of the test plate.

Compound 3 was tested in 8 points by two-fold serial titrations from 100 to 0.78 μg/mL. Rifampicin was tested in 7 points by two-fold serial titrations ranged from 12.8 to 0.2 μg/mL. Assay plates were incubated and MIC endpoints were read as complete inhibition of visual growth. The checkerboard combination analysis was performed following the MIC testing protocol to identify potential synergistic, indifferent or antagonistic interactions. The analysis also included each test substance alone at the same concentration ranges.

FIC (Fraction Inhibitory Concentration) and FIC Index (FICI) values for each test condition and the average of FICI values of all pairwise combinations were calculated to determine if synergy or antagonism existed between the two substances. The FIC index was determined by calculating the sum of the ratios of MICs (minimum inhibitory concentrations) for both substances. Arithmetically, the FIC Index of a combination of rifampicin and compound 3 was defined as followings: FICI=Σ[FIC (Substance 1)+FIC (Substance 2)]=[(MIC of Substance 1 in combination/MIC of Substance 1 alone)+(MIC of Substance 2 in combination/MIC of Substance 2 alone)]. Synergy is defined as the FICI (Σ)≤0.5; additivity or indifference are defined as the FICI (Σ)>0.5 to ≤4; antagonism is defined as the FICI (Σ)>4. The two fold of highest test article concentration was used for FIC value calculations in the circumstances where MIC values were greater than the highest test article concentration.

In the 17 checkerboard assays, the fractional inhibitory concentration (FIC) values were calculated for each combination, and each FIC index (FICI) was calculated to assess synergistic, indifferent or antagonistic interactions. MIC values of compound 3 and rifampicin ranged from 6.25 to >100 μg/mL, and 3.125 to >12.5 μg/mL, respectively, for the 17 test strains. The average of FICI of compound 3 in combination with rifampicin ranged from 0.19 to 1.03 (Table 2). Compound 3 in combination of rifampicin resulted in synergistic effects for most tested strains (see Table 2).

TABLE 2
Checkerboard Assay Summary

| | | | MIC of | MIC of | | |
|---|---|---|---|---|---|---|
| | | | *Cmpd 3 | **Rifa. | FICI | Combination |
| | Strains | Strain No. | (ug/mL) | (ug/mL) | (Average) | Result |
| 1 | Serratia marcescens | ATCC 13880 | >100 | >12.5 | 0.21 | synergy |
| 2 | Salmonella typhimurium | ATCC13311 | 12.5 | >12.5 | 0.34 | synergy |
| 3 | Salmonella choleraesuis | ATCC 10708 | 50 | 12.5 | 0.32 | synergy |
| 4 | Acinetobacter | BAA-1605 | 25 | 3.125 | 0.31 | synergy |
| | baumannii | | | | | |
| 5 | Acinetobacter | ATCC 17978 | 25 | 6.25 | 0.27 | synergy |
| | baumannii | | | | | |
| 6 | Citrobacter freundii | ATCC 8090 | 100 | >12.5 | 0.19 | synergy |
| 7 | Pseudomonas | BCCM27647 | 25 | >12.5 | 1.03 | additivity/ |
| | aeruginosa | | | | | indifference |
| 8 | Pseudomonas | BCCM27648 | 25 | >12.5 | 1.03 | additivity/ |
| | aeruginosa | | | | | indifference |
| 9 | Escherichia coli | ATCC 25922 | 6.25 | 6.25 | 0.52 | additivity/ |
| | | | | | | indifference |
| 10 | Escherichia coli | ATCC 10536 | 6.25 | 3.125 | 0.48 | synergy |
| 11 | Stenotrophomanas | ATCC 13637 | 12.5 | 6.25 | 0.39 | synergy |
| | maltophlia | | | | | |
| 12 | Enterobacter cloacae | BAA-1143 | 100 | >12.5 | 0.19 | synergy |
| 13 | Enterobacter cloacae | ATCC-13047 | 100 | >12.5 | 0.19 | synergy |
| 14 | Enterobacter aerogens | ATCC-13048 | 100 | >12.5 | 0.19 | synergy |
| 15 | Acinetobacter | AR-BANK | 25 | 3.125 | 0.31 | synergy |
| | baumannii | 0088 | | | | |
| 16 | Klebsiella pneumoniae | NCTC-13438 | 50 | >12.5 | 0.22 | synergy |
| 17 | Klebsiella pneumoniae | AR- | 100 | >12.5 | 0.19 | synergy |
| | | BANK#0160 | | | | |

*Cmpd 3: compound 3 (μg/mL)
**Rifa.: Rifampicin (μg/mL)

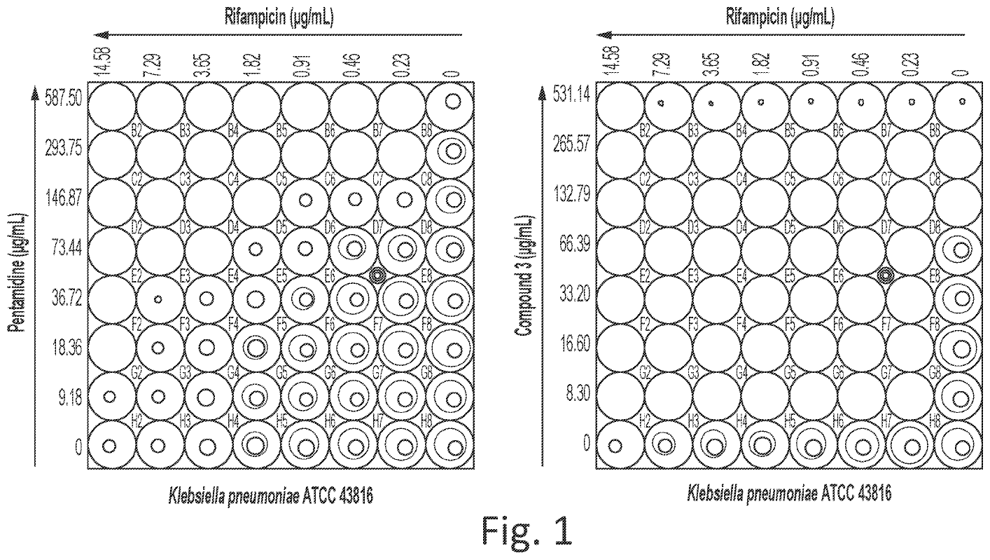

A representative image of a checkerboard assay is shown in FIG. 1. A synergistic interaction between compound 3 and rifampicin is depicted on a multi-well plate having concentrations of these two compounds tested alone (0 μg/mL) and in combination, ranging from 531 to 8.3 μg/mL for compound 3 and from 14.58 to 0.23 μg/mL for rifampicin. As control, pentamidine was used for its synergistic, indifferent or antagonistic interaction with rifampicin in similar conditions. As demonstrated in FIG. 1, compound 3 exhibited superior synergistic effects with rifampicin than pentamidine with rifampicin in exerting growth inhibition on K. pneumoniae (FIG. 1). In FIG. 1, dark wells indicated growth inhibition of K. pneumoniae and corresponding antibacterial activities of these compounds.

Rifampicin is an antibiotic drug used for the treatment of infections caused by such serious pathogens as Legionella, Mycobacterium tuberculosis and Mycobacterium avium complex (MAC) that mainly affect the lungs and cause pulmonary infections; and Mycobacterium leprae that causes leprosy. Resistance to rifampicin occurs due to mutations developed in this strain. Rifampicin is also known to be effective against Neisseria meningitidis, a gram negative bacterium that causes meningitis and other forms of meningococcal disease.

In addition to rifampicin, another antibacterial drug, novobiocin, was used to test its potential synergistic effect with any of compounds 1-3. Novobiocin is an anti-staphylococcal drug, but with limited use. The oral form of novobiocin drug has since been withdrawn from the market due to lack of efficacy. This antibiotic is often used to treat Staphylococcus aureus infection, particularly methicillin resistant S. aureus “MRSA”.

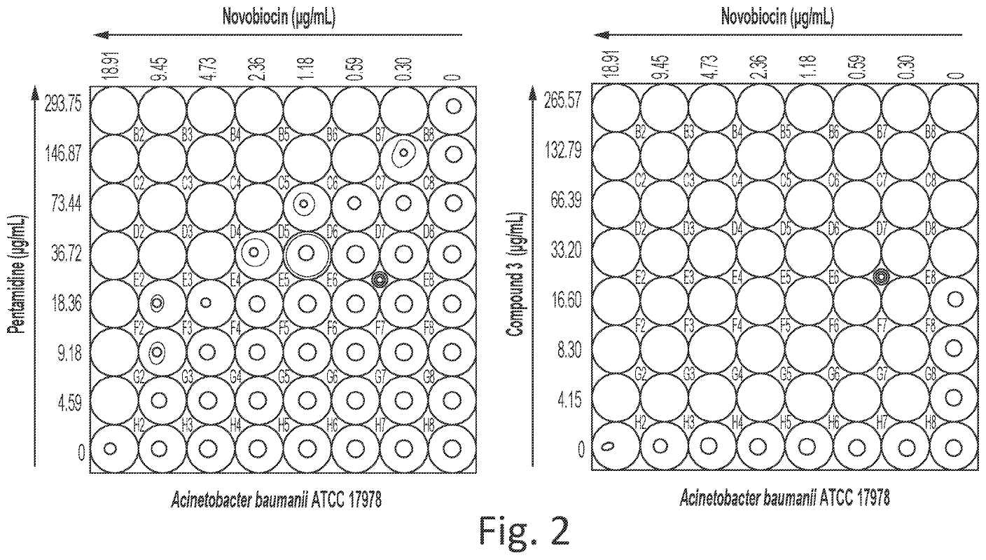

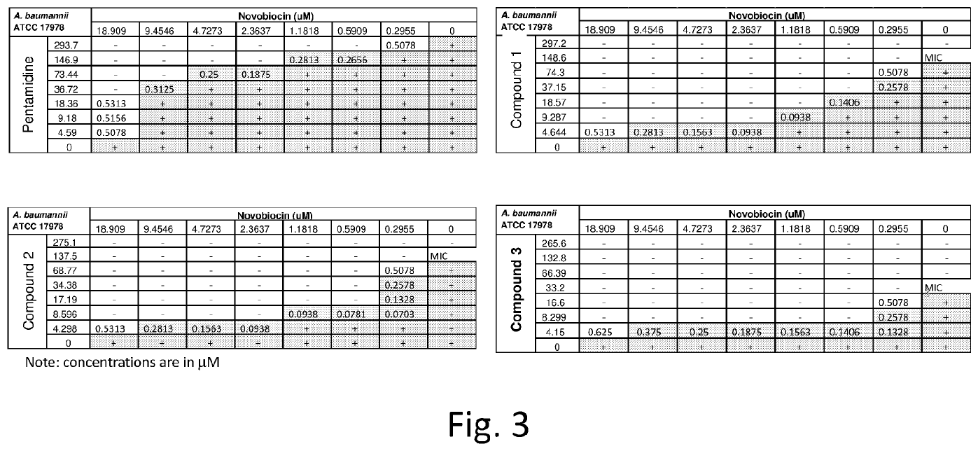

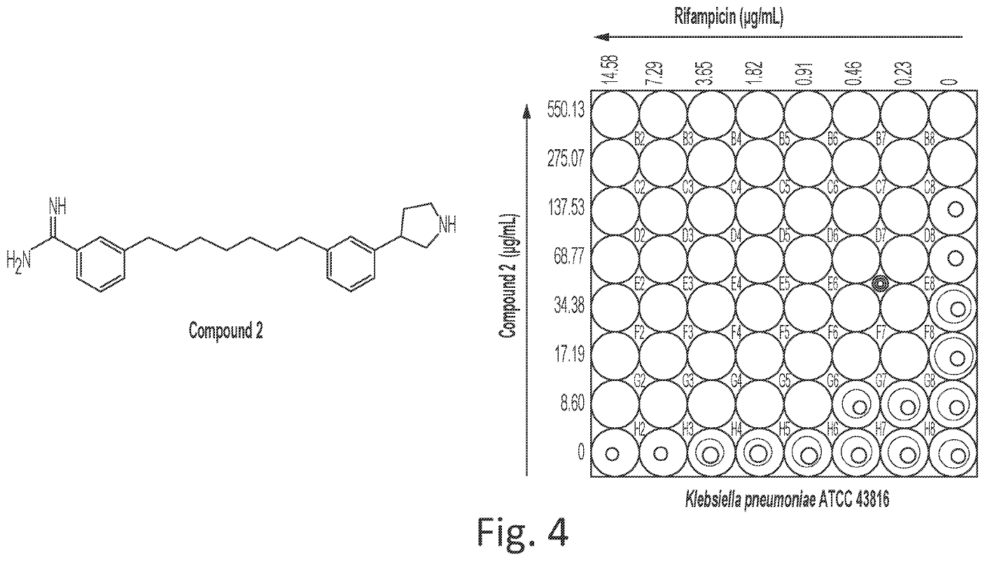

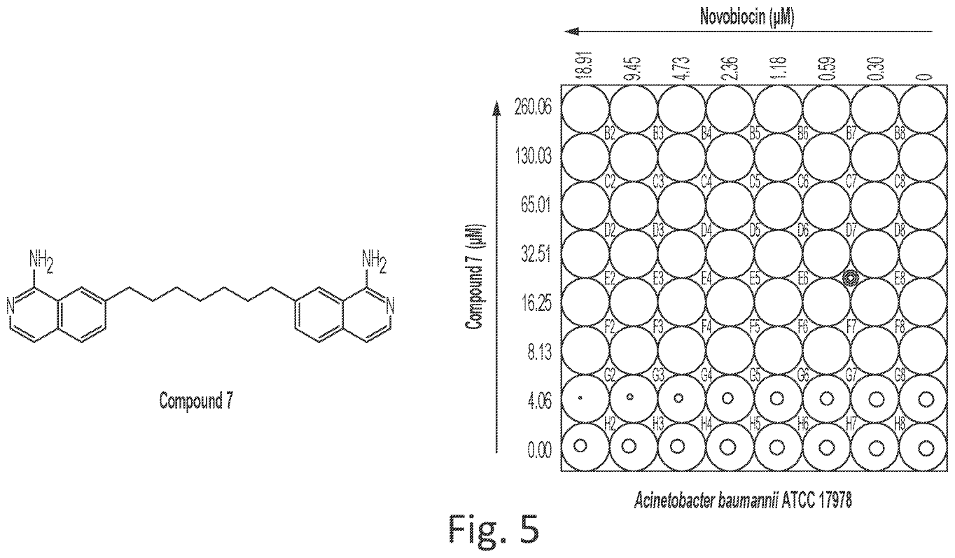

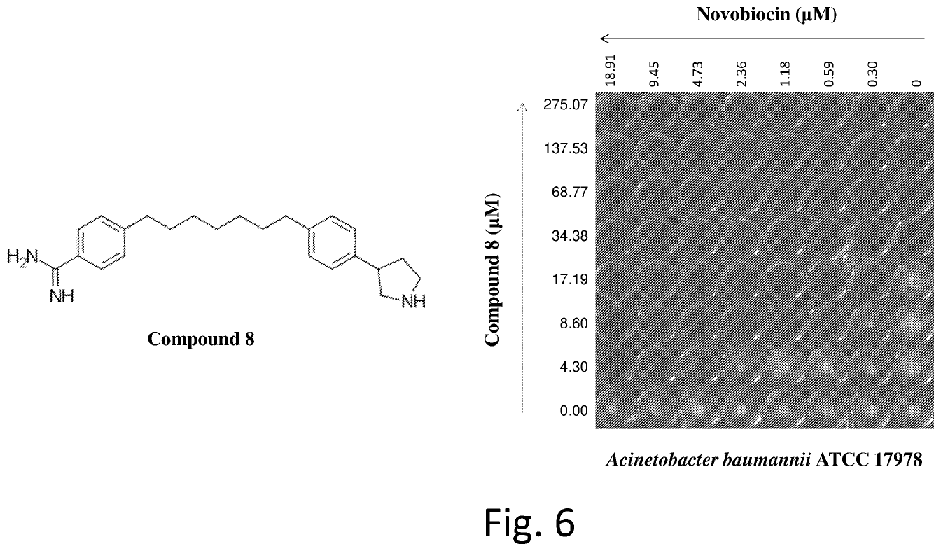

Compounds 1, 2 and 3 of the present invention combined with novobiocin exhibited superior synergy in inhibiting Acinetobacter baumannii as compared to pentamidine with novobiocin. Particularly, MIC of compound 3 was 33.2 μM, while MIC of novobiocin is >18.9 μM. When FICI is calculated, MIC of novobiocin appeared to be 37.8 μM. The clear synergistic effect was observed between compound 3 and novobiocin against A. baumannii ATCC 17978 (FIGS. 2 and 3). Further, MIC of compound 1 and compound 2 were 148.6 μM and 137.5 μM, respectively, while that of novobiocin was >18.9 μM as mentioned above. There were clear synergistic effects observed in compounds 1 and 2 with novobiocin against A. baumannii ATCC17978 (FIG. 3). Compound 2 also showed increased level of synergy with rifampicin against K. pneumoniae ATCC 43816 (FIG. 4). Interestingly, compound 7's MIC was observed 8.13 μM, while this compound showed no synergistic effect with novobiocin against A. baumannii ATCC 17987 (FIG. 5). This result suggested that compound 7 is an antibacterial agent by itself at 8.13 μM. MIC of compound 8 was >34.4 μM and the compound showed increased synergy with novobiocin against A. baumannii ATCC 17978 as compared to pentamidine (FIG. 6).

### Example 9. Pharmacokinetic Analysis

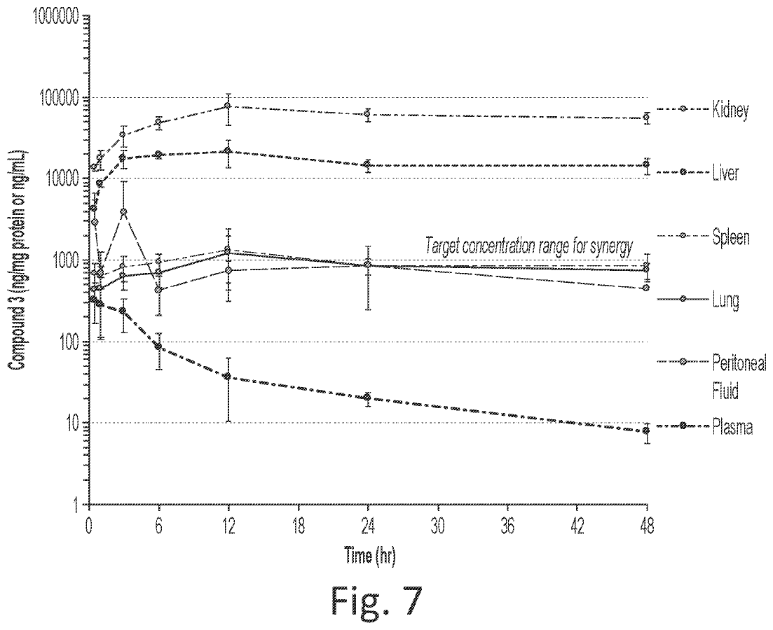

To determine whether target concentrations/exposures of these compounds can be achieved at various target organs, exposures of compound 3 were tested in kidney, liver, spleen, lung peritoneal fluid and plasma of male C57BL6 mice (5 mice per group). Compound 3 was dosed at 10 mg/kg subcutaneously with each group having samples taken at time indicated (FIG. 7). Based on the in vitro antibacterial studies, compound 3's target concentrations for pharmacology were approximately 2.5 to 5 μM in combination with either novobiocin or rifampicin. As shown in FIG. 7, the exposure of compound 3 in mice showed that target concentrations for pharmacology could be achieved at least with 10 mg/kg subcutaneous administration in kidney, liver, and lung (FIG. 7). These results suggest that, with higher doses (e.g., >10 mg/kg), compound 3 could be readily available in other major organs above pharmacological concentrations.

### Example 10. Gram Positive Antibacterial Testing

The minimally effective concentrations (MIC; lowest concentration of a test agent that will inhibit the visible growth of a microorganism after overnight incubation) of compounds 3 and 5, as well as control reference agents pentamidine and linezolid, against 2 S. aureus strains (ATCC 43300 and ATCC 27660) were determined. compound 3 had a MIC of 0.5 μg/mL in both strains, which was the lowest observed MIC value. By contrast, pentamidine had a MIC of 64 μg/mL in ATCC 43300 and 16 μg/mL in ATCC 27660. Linezolid was used as a control and had a MIC value of 4 μg/mL in both strains.

Compounds were reconstituted into DMSO or suggested solvent to 12.8 mg/ml. Then the compound stocks were serially 2-fold diluted in a v-bottom 96-well plate to make 100-fold working solutions. One day before the test, −80° C. glycerol stock of S. aureus strains were streaked on cation-adjusted Mueller-Hinton agar (CAMHA) plates. On the day of test, fresh bacterial colonies were suspended into sterile saline to an OD600 nm of ˜0.1 (equivalent to 0.5 McFarland). The bacterial suspension was firstly 20-fold followed by 50-fold diluted in cation-adjusted Mueller-Hinton broth (CAMHB) plates to 1×10\hat{ }6 CFU/PO. 198 ul of the inoculum was added into the round-bottom 96-well plates prefilled with 2 ul of 100-fold compound working solutions. After incubating at 35 +/−2° C. in ambient atmosphere for 18-24 h, MIC was recorded as the lowest concentration under which no visible bacterial growth was observed.

TABLE 3
MIC Assay Against Gram Positive S aureus
MIC (in μg/mL)

| | Strain name | |
|---|---|---|
| | S. aureus | S. aureus |
| Compound | ATCC 43300 | ATCC 27660 |
| Compound 3 | 0.5 | 0.5 |
| Compound 5 | 8 | 8 |
| Pentamidine | 64 | 16 |
| Linezolid | 4 | 4 |

### Example 11. Additional Gram Positive Antibacterial Testing

The MIC values of test article compound 3, as well as control reference agents, vancomycin and linezolid, are summarized in the below table for S. aureus strains ATCC 19636, ATCC 33591, BAA-1556 and VRS-2. The MICs of vancomycin and linezolid were determined as quality controls and the MIC values of each strain met the acceptance criteria based on PDS historical reference data. The MIC values of compound 3 against the four S. aureus strains ranged from 0.25 to 0.125 μg/mL. Strain S. aureus VRS-2, with an compound 3 MIC of 0.25 μg/mL, was selected for the peritonitis infection model.

The direct colony suspension method was used to prepare inoculated broth. Isolated colonies were taken from an 18-24 h culture plate. Optical density measurements (OD620 nm) were used to estimate the bacterial density. The test article, compound 3, and reference control stock solutions were prepared in 100% DMSO as 50× stock solutions. The 50× stock solutions were diluted by 2-fold serial titrations with 100% DMSO for a total of 11 concentrations. A 4 μL aliquot of each dilution was added to 196 μL of cation-adjusted Mueller Hinton Broth II medium seeded with the organism in wells of a 96 well plate. The final vehicle concentration was two percent (2%) in all assay wells. The final bacterial count was 2 to 8×105 CFU/mL. The final compound 3 concentration range was 16 to 0.016 μg/mL. Each test substance dilution was evaluated in duplicate on one test occasion. Vehicle-control and reference controls were used as blank and positive controls, respectively. Plates were incubated at 35-37° C. for 18 h. The test plates were visually examined and each well was visually scored for growth or complete inhibition of growth. The MIC value was recorded. The Clinical and Laboratory Standards Institute (CLSI) guidelines of 100% visual growth inhibition were used to call an MIC endpoint.

TABLE 4
MIC Assay Against Gram Positive and Antibiotic-Resistant S. aureus

| | | | | | MIC, μg/mL | | |
|---|---|---|---|---|---|---|---|
| | | | | | | Reference | |
| | Assay | | | Infor- | *Cmpd | Vanco- | Line- |
| No. | # | Species | Strain ID | mation | 3 | mycin | zolid |
| 1 | 606000 | Staphylococcus aureus | ATCC 19636 | — | 0.125 | 0.5 | 4 |
| 2 | 605000 | Staphylococcus aureus | ATCC 33591 | MRSA | 0.125 | 0.5 | 2 |
| 3 | 604055 | Staphylococcus aureus | BAA-1556 | MRSA | 0.25 | 0.5 | 2 |
| 4 | 604147 | Staphylococcus aureus | VRS-2 | VRSA | 0.25 | >64 | 4 |

*Cmpd 3: compound 3
MRSA: methicillin-resistant Staphylococcus aureus.
VRSA: vancomycin-resistant Staphylococcus aureus.

### Example 12. In Vivo VRS-2 Model

The efficacy of compound 3 was evaluated in the S. aureus VRS-2 peritonitis infection model. The test article, compound 3 at 0.1, 0.3 and 1 mg/kg, was intraperitoneally (IP) administered twice at 2 and 16 h after infection; as well as once at 2 mg/kg at 2 h post-infection. The control reference agent, linezolid at 50 mg/kg, was orally (PO) administered once at 1 h post-infection. Animal mortality was observed for 7 consecutive days. All animals in the vehicle group succumbed to infection between Day 1 to Day 2, resulting in 100% mortality, and a mean 15 h survival time. A full protective effect was observed in animals treated with the reference control, 50 mg/kg PO QD, resulting in 100% survival rate (p<0.05) with a 180 h mean survival time (p<0.05). In the compound 3 at 1 mg/kg IP BID dose group, one out of eight animals survived till Day 7 of the study period, resulting in 13% survival rate, and a mean 33 h survival time. The IP BID administrations of compound 3 at lower doses of 0.1 and 0.3 mg/kg, as well as at a single dose of 2 mg/kg all resulted in 100% mortality with a mean survival time ranging from 15 h to 18 h.

For efficacy testing, the 0.4 mg/mL compound 3 solution was prepared by dissolving 1.21 mg of compound 3, corresponding to active ingredient of 1.02 mg with a correction factor of 1.19, in 2.55 mL of WFI. The 0.4 mg/mL compound 3 working solution was further diluted with WFI to generate three lower dosing solutions of 0.2, 0.06 and 0.02 mg/mL. All dosing solutions were soluble and colorless.

PDS supplied vancomycin and linezolid as reference controls for both MIC and in vivo studies. For MIC testing, the 3.2 mg/mL vancomycin 50× stock solution was prepared by dissolving 1.2 mg of vancomycin in 0.375 mL of WFI. The 3.2 mg/mL linezolid 50× stock solution was prepared by dissolving 1.0 mg of linezolid in 0.313 mL of DMSO. Both solutions were colorless and soluble. For efficacy testing, one tablet of linezolid, at 600 mg, was dissolved in 12 mL of 1% Tween 80 in saline (0.9% NaCl) to generate a 50 mg/mL of stock solution. The 50 mg/mL linezolid stock solution was further diluted with 1% Tween 80 in 0.9% NaCl to generate the dosing solution of 5 mg/mL. Both solutions were insoluble with white color.

The S. aureus strain VRS-2 peritonitis infection model was performed with immunocompetent female ICR mice, weighing 22±2 g. All animals were specific pathogen free. The PDS standard protocol for preparing S. aureus cultures for mouse infection studies was followed. A 0.2 mL aliquot of a single-use glycerol stock (at −80° C.) was used to seed mL brain heart infusion (BHI) and then incubated at 35-37° C. with shaking (250 rpm) for 8 h. Bacterial cells in the 20 mL was pelleted by centrifugation (3,500×g) for 15 minutes, and then re-suspended in 10 mL cold phosphate buffer saline (PBS). The optical density, OD620 nm, was measured and used to guide dilution. The PBS suspensions were stored on ice for no more than 1 h prior to animal inoculation. The dilutions were performed by 1:1 dilution of BHI broth containing hog gastric mucin to obtain the target inoculum density, 1×108 CFU/mouse, per 0.5 mL with 5% mucin (final concentration). The actual colony count was determined by plating dilutions to nutrient agar (NA) plates followed by 20-24 h incubation at 35-37° C.

On Day 0, animals were infected with bacterial suspension with 5% mucin by intraperitoneal injection. The actual inoculum was 1.93×108 CFU/mouse with a target inoculum of 1×108 CFU/mouse in the peritonitis infection model. Test article, compound 3, was formulated in WFI and intraperitoneally (IP) administered with the dose schedule and concentrations indicated in the study design. Doses were administered once at 2 h post-infection or twice (BID) at 2 and 16 h post-infection. The animals were monitored for survival up to 7 days, twice daily (AM 9:00/PM 17:00). Control reference agent, linezolid at mg/kg, was formulated in 1% Tween 80 in saline, and orally administered at 1 h post-infection. Animals were observed for 30 min after dosing to detect acute toxicity which was recorded and reported, if observed. Animals were humanely euthanized if severe acute toxicity was observed.

Animals were monitored for mortality twice daily (AM 9:00/PM 17:00) for 7 days after infection. Survival (percentage) of animals was plotted as a function of time using the Kaplan Meier method using GraphPad Prism. Fisher's exact test was used to assess the statistical significance in the survival of the treated animals compared to the vehicle control group at the day 7 time point. The p value<0.05 indicates a significant increase in the survival rate of the treated animal group. The mean survival time was calculated with 12 h intervals with the maximum survival time set at 180 h (7.5 days). Student's t-test was used to assess the statistical significance in the mean survival time of the treated animals compared to the vehicle control group.

### Example 13. Effect of Compound 3 on Cell Culture Viability of Human Primary Hepatocytes

Compound 3 was tested for cytotoxicity in human primary hepatocytes in duplicate at 8 concentrations ranging from 0.03 μM to 100 μM (0.013 μg/ml to 44.95 μg/ml ). The IC50 of compound 3 was determined after 24 hours to be 25 μM (11.24 μg/ml). No inhibition on cell viability was observed at 10 μM (4.49 μg/ml) and lower concentrations. Compound 3 demonstrated significant effects on cell viability at 30 uM and 100 μM (13.48 μg/ml and 44.95 μg/ml).

This assay was performed using cryopreserved human primary hepatocytes (PHH). The cryopreserved PHH were seeded in collagen I coated 96-well plates in plating medium. The medium was replaced with incubation medium 2-4 hours after seeding. After overnight culture, test compound (compound 3) (0.03, 0.1, 0.3, 1, 3, 10, 30, 100 μM), reference compound (chlorpromazine) and vehicle control (1% DMSO) were added, and incubated with the cells for 24 hours followed by the addition of the alamarBlue cell viability reagent. The plates are analyzed for fluorescent intensity after alamarBlue addition. % of cell viability (% of control) was calculated by comparing the background (wells without cells) corrected fluorescent intensity in the compound treated wells and vehicle control treated wells. IC50 values were determined by non-linear regression analysis of the concentration-response curves using the Hill equation. Testing was done in duplicate.

### Example 14. Anti-fungal Activity of Compound 3

Compound 3 MIC was determined at both 50% growth inhibition and 100% growth inhibition in 2 rounds of anti-fungal testing. The first round of testing included Candida parapsilosis, Candida krusei, Paecilomyces variotii, Candida albicans, Aspergillus fumigatus, and Blastomyces dermatitidis. Isolate MIC (100%) for compound 3 ranged from 1 μg/ml (Candida parapsilosis, Paecilomyces variotii, Candida albicans) to 64 μg/ml (Aspergillus fumigatus). The second round of testing included Candida parapsilosis, Candida krusei, Candida albicans, Candida auris, Candida glabrata, Candida guilliermondii, and Cryptococcus neoformans. Isolate MIC (100%) for compound 3 ranged from 0.5 μg/ml (Candida parapsilosis, Candida albicans, Cryptococcus neoformans) to 8 μg/ml (Candida parapsilosis). Not only is compound 3 effective in killing many species of fungus, it is also more effective in killing several Candida strains of interest that are not affected by fluconazole.

Stock solutions of the investigational agent were prepared at concentrations 100-times the highest concentration to be tested (6400 μg/ml) using dimethyl sulfoxide. Aliquots of the stock solutions were dispensed into polystyrene vials and stored at −20° C.

The synthetic medium RPMI-1640 (with glutamine, without bicarbonate, and with phenol red) was used. The RPMI was buffered to a pH of 7.0+0.1 at 25° C. with 0.165M MOPS (3-[N-morpholino] propanesulfonic acid). Sterile U-shaped 96-well cell culture plates were used to perform the MIC assays. Dilutions of the DMSO stocks were prepared in RPMI to achieve 2× concentrations. After the dilutions of the working 2× investigational antifungal solutions were prepared, 0.1 ml of each concentration was transferred into a pre-specified column of the U-shaped 96-well cell culture plate using sterile pipettes.

Fungal isolates were grown on Sabouraud dextrose agar (yeasts) or potato flake agar (filamentous fungi) and cells were collected after an appropriate period of growth for each species being evaluated (e.g., 48-72 hours for yeasts and 7 days for filamentous fungi). The fungi were suspended in sterile distilled water, and the densities of the fungal suspension were read using a spectrophotometer and adjusted to an appropriate optical density specific for each fungal species. The fungal suspension of each isolate was then diluted in RPMI. A sufficient volume of the test inoculum was prepared to directly inoculate 0.1 ml into each test well of the 96-well cell culture plate. Final inoculum ranges were dependent on the fungal species to be tested (e.g., 0.4×103 to 5×103 cells/ml for yeasts and dimorphic fungi, and 0.4×104 to 5×104 cells/ml for filamentous fungi). Each well of the 96-well cell culture plate containing the investigational antifungal compounds (0.1 ml volume) was inoculated on the day of the assay with 0.1 ml of the fungal suspension. Growth control wells contained 0.1 ml of fungal suspension and 0.1 ml of the growth medium without antifungal agents. The media control well contained 0.2 ml of the growth medium.

The microdilution trays were incubated at 35° C. without agitation. After the appropriate period of incubation (24 hours for Candida, 48 hours for Aspergillus, and 48-72 hours for Blastomyces) the trays and microdilution tubes were removed and the MIC values determined. Two MIC values were assigned to the investigational agent: 1) the concentration resulting in a prominent reduction in growth (50% inhibition compared to the growth control), and 2) the concentration resulting in complete inhibition of growth (100% inhibition vs. growth control). One positive comparator/control was used for yeast (fluconazole) and one was used for filamentous fungi (voriconazole).

TABLE 5
Anti-fungal activity of compound 3

| | | Compound 3 | | Fluconazole | | Voricolazole | |
|---|---|---|---|---|---|---|---|
| Species | Isolate | 50% | 100% | 50% | 100% | 50% | 100% |
| Candida | ATCC22019 | 0.5 | 1 | 1 | 2 | — | — |
| parapsilosis | | | | | | | |
| Candida | ATCC6258 | 8 | 8 | — | — | ≤0.03 | 0.125 |
| krusei | | | | | | | |
| Paecilomyces | MYA-3630 | 1 | 1 | 16 | 32 | — | — |
| variotii | | | | | | | |
| Candida | SC5314 | 1 | 1 | 0.25 | >64 | — | — |
| albicans | ATCC90028 | 0.5 | 1 | ≤0.125 | >64 | | |
| | CA3 | 1 | 1 | >64 | >64 | — | — |
| Aspergillus | AF293 | 16 | 16 | — | — | 0.25 | |
| fumigatus | AF2 | 32 | 32 | — | — | >16 | >16 |
| | AF3 | 32 | 64 | — | — | 2 | 4 |
| Blastomyces | BD1 | 4 | 8 | — | — | ≤0.03 | 0.06 |
| dermatitidis | BD2 | 2 | 4 | — | — | ≤0.03 | 0.125 |
| | BD3 | 4 | 8 | — | — | ≤0.03 | 0.125 |

TABLE 6
Anti-fungal activity of compound 3

| | | Compound 3 | | Fluconazole | |
|---|---|---|---|---|---|
| Species | Isolate No. | 50% | 100% | 50% | 100% |
| C. parapsilosis | ATCC 22019 | 0.25 | 0.5 | 2 | 2 |
| C. krusei | ATCC 6258 | 0.5 | 1 | 16 | 32 |
| Candida | 43001 | 0.25 | 0.5 | >64 | >64 |
| albicans | CA5 | 0.25 | 0.5 | 0.25 | >64 |
| | CA6 | 0.5 | 0.5 | 0.25 | >64 |
| Candida auris | DI17-47 | 1 | 4 | >64 | >64 |
| | DI17-48 | 2 | 4 | 2 | 16 |
| | DI17-46 | 2 | 4 | >64 | >64 |
| Candida | 05-62 | 2 | 4 | >64 | >64 |
| glabrata | 05-761 | 1 | 2 | 8 | 32 |
| | CG3 | 0.5 | 1 | 0.5 | 64 |
| Candida | Cgui1 | 1 | 1 | 1 | 4 |
| guilliermondii | Cgui2 | 2 | 2 | 2 | 4 |
| | Cgui3 | 2 | 2 | 2 | 4 |
| Candida | CP3 | 2 | 8 | 1 | 1 |
| parapsilosis | CP2 | 1 | 4 | 0.5 | 0.5 |
| | CP1 | 1 | 8 | 0.5 | 1 |
| Cryptococcus | USC 1597 | 0.5 | 0.5 | 4 | 8 |
| neoformans | H99 | 0.5 | 0.5 | 16 | 32 |
| | CN3 | 0.5 | 0.5 | >64 | >64 |

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled. Such modifications are intended to fall within the scope of the appended claims.

All references, patent and non-patent, cited herein are incorporated herein by reference in their entireties and for all purposes to the same extent as if each individual publication or patent or patent application was specifically and individually indicated to be incorporated by reference in its entirety for all purposes.

What is claimed is:

1. A compound comprising Formula (I′) 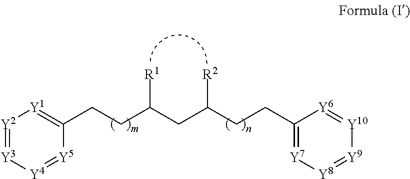
or a stereoisomer or pharmaceutically acceptable salt thereof, wherein:
m and n are independently 0, 1, 2 or 3;
R¹ and R² are independently hydrogen or halo, or R¹ taken together with R² forms a saturated, unsaturated or partially unsaturated 3-9 member ring; and
Y¹ through Y¹⁰ are independently CR³, wherein R³ is independently hydrogen, heterocycle, or amidine, or R³ taken together with another R³ at an immediately adjacent carbon atom forms 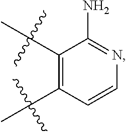 [SMILES: *c1ccnc(N)c1*]
wherein:
R³ at Y¹, Y⁵, Y⁶ and Y⁷ is hydrogen, and
R³ at one of Y², Y³ or Y⁴ and optionally at one of Y⁸, Y⁹, or Y¹⁰ is amidine, or taken together with another R³ at an immediately adjacent carbon atom forms 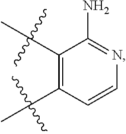 [SMILES: *c1ccnc(N)c1*]
provided that
when R¹ taken together with R² forms the saturated, unsaturated or partially unsaturated 3-9 member ring, R³ is amidine at one of Y², Y³, or Y⁴, and R³ is amidine at one of Y⁸, Y⁹, or Y¹⁰, then m and n are independently 1, 2 or 3; and
when R¹ and R² are both hydrogen, R³ is amidine at one of Y², Y³, or Y⁴, and R³ is amidine at one of Y⁸, Y⁹, or Y¹⁰, then R³ at Y³ and R³ at Y⁹ are independently hydrogen or heterocycle.

2. The compound of claim 1, or a stereoisomer or pharmaceutically acceptable salt thereof, wherein the compound is of Formula (I) 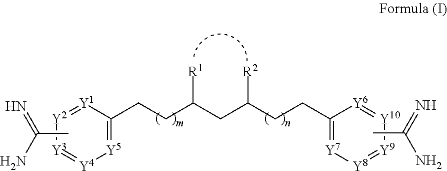
wherein:
m and n are independently 0, 1, 2 or 3;
R¹ and R² are independently hydrogen or halo, or R¹ taken together with R² forms a saturated, unsaturated or partially unsaturated 3-9 member ring; and
Y¹ through Y¹⁰ are independently CR³, wherein R³ is hydrogen at Y¹, Y⁵, Y⁶ and Y⁷, and R³ at Y², Y³, Y⁴, Y⁸, Y⁹, and Y¹⁰ is independently hydrogen, heterocycle, or absent when a corresponding carbon is attached to an amidine,
provided that
when R¹ taken together with R² forms the saturated, unsaturated or partially unsaturated 3-9 member ring, then m and n are independently 1, 2 or 3; and
when R¹ and R² are both hydrogen, then R³ at Y³ and R³ at Y⁹ are independently hydrogen or heterocycle.

3. The compound of claim 1, or a stereoisomer or pharmaceutically acceptable salt thereof, wherein the compound is of Formula (I′-a) 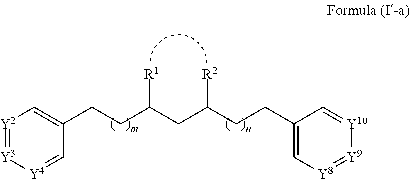
wherein:
m and n are independently 1, 2 or 3;
R¹ taken together with R² forms a saturated, unsaturated or partially unsaturated 3-9 member ring; and
Y² through Y⁴ and Y⁸ through Y¹⁰ are independently CR³, wherein R³ is independently hydrogen, heterocycle, or amidine, wherein:
R³ is amidine at one of Y², Y³ or Y⁴ and optionally at one of Y⁸, Y⁹, or Y¹⁰.

4. The compound of claim 1, or a stereoisomer or pharmaceutically acceptable salt thereof, wherein the compound is of Formula (I′-b) 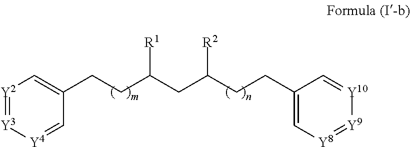
wherein:
m and n are independently 0, 1, 2 or 3;
R¹ and R² are independently hydrogen or halo; and
Y² through Y⁴ and Y⁸ through Y¹⁰ are independently CR³, wherein R³ is independently hydrogen, heterocycle, or amidine, wherein:
R³ is amidine at one of Y², Y³ or Y⁴ and optionally at one of Y⁸, Y⁹, or Y¹⁰;
provided that
when R³ is amidine at one of Y², Y³, or Y⁴, and R³ is amidine at one of Y⁸, Y⁹, or Y¹⁰, then R³ at Y³ and R³ at Y⁹ are independently hydrogen or heterocycle.

5. The compound of claim 1, or a stereoisomer or pharmaceutically acceptable salt thereof, wherein the compound is of Formula (I′-c) 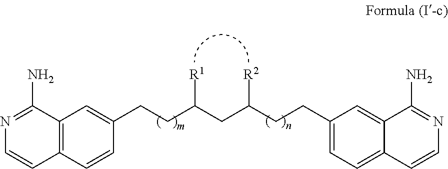
wherein:
m and n are independently 0, 1, 2 or 3;
R¹ and R² are independently hydrogen or halo, or R¹ taken together with R² forms a saturated, unsaturated or partially unsaturated 3-9 member ring.

6. The compound of any one of claims 1-5, or a stereoisomer or pharmaceutically acceptable salt thereof, wherein m is 1, and n is 1.

7. The compound of claim 1, 2, 4, or 5, or a stereoisomer or pharmaceutically acceptable salt thereof, wherein R¹ and R² are independently hydrogen.

8. The compound of claim 1, 2, 3, or 5, or a stereoisomer or pharmaceutically acceptable salt thereof, wherein R¹ taken together with R² forms 6 member saturated cycloalkyl.

9. The compound of claim 1 or 3, or a stereoisomer or pharmaceutically acceptable salt thereof, wherein R¹ taken together with R² forms a saturated, unsaturated or partially unsaturated 3-9 member ring; and R³ is amidine at Y⁴ and Y⁸ and hydrogen at Y², Y³, Y⁹, and Y¹⁰.

10. The compound of claim 9, or a stereoisomer or pharmaceutically acceptable salt thereof, wherein R¹ taken together with R² forms a saturated 6 member cycloalkyl.

11. The compound of claim 1 or 3, or a stereoisomer or pharmaceutically acceptable salt thereof, wherein R¹ taken together with R² forms a saturated, unsaturated or partially unsaturated 3-9 member ring; and R³ is amidine at Y² and Y¹⁰ and hydrogen at Y³, Y⁴, Y⁸, and Y⁹.

12. The compound of claim 11, or a stereoisomer or pharmaceutically acceptable salt thereof, wherein R¹ taken together with R² forms a saturated 6 member cycloalkyl.

13. The compound of claim 1 or 3, or a stereoisomer or pharmaceutically acceptable salt thereof, wherein R¹ taken together with R² forms a saturated, unsaturated or partially unsaturated 3-9 member ring; and R³ is amidine at Y³ and Y⁹, and hydrogen at Y², Y⁴, Y⁸, and Y¹⁰.

14. The compound of claim 13, or a stereoisomer or pharmaceutically acceptable salt thereof, wherein R¹ taken together with R² forms 6 member cycloalkyl.

15. The compound of any one of claim 1, 3, or 4, or a stereoisomer or pharmaceutically acceptable salt thereof, wherein R³ is a heterocycle at Y⁸, Y⁹, or Y¹⁰ when amidine is not present at any one of Y⁸, Y⁹, or Y¹⁰.

16. The compound of claim 1 or 4, or a stereoisomer or pharmaceutically acceptable salt thereof, wherein R¹ and R² are independently hydrogen and R³ is amidine at Y², a saturated 5 member heterocycloalkyl at Y¹⁰, and hydrogen at Y³, Y⁴, Y⁸, and Y⁹.

17. The compound of claim 1 or 4, or a stereoisomer or pharmaceutically acceptable salt thereof, wherein R¹ and R² are independently hydrogen and R³ is amidine at Y³, a saturated 5 member heterocycloalkyl at Y⁹, and hydrogen at Y², Y⁴, Y⁸, and Y¹⁰.

18. The compound of claim 1 or 3, or a stereoisomer or pharmaceutically acceptable salt thereof, wherein R¹ taken together with R² forms a saturated, unsaturated or partially unsaturated 3-9 member ring; and R³ is amidine at Y³, a saturated 5 member heterocycloalkyl at Y⁹, and hydrogen at Y², Y⁴, y⁸, and Y¹⁰.

19. The compound of any one of claims 15-18, or a stereoisomer or pharmaceutically acceptable salt thereof, wherein the saturated 5 member heterocycloalkyl is pyrrolidinyl.

20. The compound of claim 18, or a stereoisomer or pharmaceutically acceptable salt thereof, wherein R¹ taken together with R² forms a saturated 6 member cycloalkyl.

21. The compound of claim 1, or a stereoisomer or pharmaceutically acceptable salt thereof, wherein the compound is selected from the group consisting of:
3,3′-(heptane-1,7-diyl)dibenzimidamide;
3 -(7-(3 -(pyrrolidin-3 -yl)phenyl)heptyl)benzimidamide;
4,4′-(2,2-((1R,3S)-cyclohexane-1,3-diyl)bis(ethane-2,1-diyl))dibenzimidamide;
4,4′-((2,2′-((1R,3R)-cyclohexane-1,3-diyl)bis(ethane-2,1-diyl))dibenzimidamide;
4-(2-((1S ,3R)-3-(4-(pyrrolidin-3-yl)phenethyl)cyclohexyl)ethyl)benzimidamide;
3,3′-(((1R,3S)-cyclohexane-1,3-diyl)bis(ethane-2,1-diyl))dibenzimidamide;
7,7′-(heptane-1,7-diyl)bis(isoquinolin-1-amine); and
4-(7-(4-(pyrrolidine-3-yl)phenyl)heptyl)benzimidamide.

22. A pharmaceutical composition comprising a compound of any one of claims 1-21, or a stereoisomer or pharmaceutically acceptable salt thereof, and a pharmaceutically acceptable carrier or excipient.

23. A method of treating a bacterial infection, the method comprising administering an effective amount of a compound, or a stereoisomer or pharmaceutically acceptable salt thereof, of any one of claims 1-21, or a pharmaceutical composition of claim 22, to a subject suffering from a bacterial infection.

24. A method of treating a fungal infection, the method comprising administering an effective amount of a compound or a stereoisomer or pharmaceutically acceptable salt thereof, of any one of claims 1-21, or a pharmaceutical composition of claim 22, to a subject suffering from a fungal infection.

25. The method of claim 23, wherein said bacterial infection is a gram negative bacterial infection.

26. The method of claim 23, wherein said bacterial infection is a gram positive bacterial infection.

27. The method of claim 23, wherein said bacterial infection is a bacterial infection caused by a strain selected from the group consisting of Serratia marcescens; Salmonella typhimurium, Salmonella choleraesuis, Acinetobacter baumannii, Citrobacter freundii, Pseudomonas aeruginosa; Stenotrophomonas maltophilia, Enterobacter cloacae, Enterobacter aerogene, Mycobacterium tuberculosis, Mycobacterium leprae, Mycobacterium avium complex, Staphylococcus aureus, Neisseria meningitidis, Klebsiella pneumoniae, and Klebsiella pneumoniae.

28. The method of claim 24, wherein said fungal infection is a fungal infection caused by a fungi selected from the group consisting of Candida parapsilosis, Candida krusei, Paecilomyces variotii, Candida albicans, Aspergillus fumigatus, Blastomyces dermatitidis, Candida auris, Candida glabrata, Candida guilliermondii, and Cryptococcus neoformans.

29. The method of any one of claims 23-28, wherein said subject is a human patient.

30. The method of claim 23, wherein said subject is suffering from a methicillin-resistant Staphylococcus aureus (MSA) infection, tuberculosis, or meningitidis.

31. The method of claim 23, wherein said compound is administered to the human patient via inhalation using aerosol.

32. The method of any one of claims 23-29, wherein said subject is suffering from a lung infection.

33. The method of claim 23, wherein said subject is a human patient suffering from a gram negative bacterial infection and being treated with an antibiotic drug.

34. The method of claim 23, wherein said subject is a human patient suffering from a gram positive bacterial infection and being treated with an antibiotic drug.

35. The method of claim 33 or 34, wherein said antibiotic drug is novobiocin.

36. The method of claim 33 or 34, wherein said antibiotic drug is rifampicin.

37. The method of claim 33 or 34, wherein said antibiotic drug is selected from the group consisting of novobiocin, rifampicin, cephalosporins, fluoroquinolones, aminoglycosides, imipenem, broad-spectrum penicillins with or without β-lactamase inhibitors, trimethoprim-sulfamethoxazole, glycopeptides, chloramphenicol, ansamycins, streptogramins, sulfonamides, tetracyclines, macrolides, oxazolidinones, quinolones, and lipopeptides.

38. The method of claim 37, wherein said antibiotic drug is selected from the group consisting of ceftriaxone-cefotaxime, ceftazidime, ciprofloxacin, levofloxacin, gentamicin, amikacin, amoxicillin-clavulanic acid, piperacillin-tazobactam, vancomycin, teicoplanin, geldanamycin, pristinamycin IIA, pristinamycin IA, prontosil, sulfanilamide, sulfadiazine, sulfisoxazole, tetracycline, doxycycline, limecycline, oxytetracycline, erythromycin, clarithromycin, azithromycin, linezolid, posizolid, tedizolid, cycloserine, ciprofloxacin, levofloxacin, trovafloxacin, daptomycin, and surfactin.

39. The method of claim 24, wherein said subject is a human patient suffering from a fungal infection and being treated with an antifungal drug.

40. The method of claim 39, wherein said antifungal drug is selected from the group consisting of azoles, polyenes, echinocandins, and flucytosine.

41. The method of claim 40, wherein said antifungal drug is selected from the group consisting of imidazole, ketoconazole, clomitrazole, fluconazole, itraconazole, posaconazole, voriconazole, isavuconazole, amphotericin B, nystatin, nidulafungin, caspofungin, and micafungin.
